(19) United States
(12) Patent Application Publication
(10) Pub. No.: US 2022/0251134 A1
(43) Pub. Date: Aug. 11, 2022

(54) 2,4,7-SUBSTITUTED-7-DEAZA-2'-DEOXY-2'-FLUOROARABINOSYL NUCLEOSIDE AND NUCLEOTIDE PRO-DRUGS AND USES THEREOF

(71) Applicant: Southern Research Institute, Birmingham, AL (US)

(72) Inventors: Omar Moukha-Chafiq, Hoover, AL (US); Larry D. Bratton, Birmingham, AL (US); Corinne E. Augelli-Szafran, Homewood, AL (US); Mark J. Suto, Homewood, AL (US)

(21) Appl. No.: 17/618,489

(22) PCT Filed: Jun. 12, 2020
(86) PCT No.: PCT/US2020/037588

§ 371 Date: Dec. 12, 2021

Related U.S. Application Data
(60) Provisional application No. 62/861,837, filed on Jun. 14, 2019.

(51) Int. Cl. C07H 19/14

(52) U.S. Cl. CPC C07H 19/14

(57) ABSTRACT

The present disclosure is concerned with 2,4,7-substituted-7-deaza-2′-deoxy-2′-fluoroarabinosyl nucleoside and nucleotide prodrugs that are capable of inhibiting viral infections and methods of treating viral infections such as, for example, human immunodeficiency virus (HIV), human papillomavirus (HPV), herpes simplex virus (HSV), human cytomegalovirus (HCMV), chicken pox, infectious mononucleosis, mumps, measles, rubella, shingles, ebola, viral gastroenteritis, viral hepatitis, viral meningitis, human metapneumovirus, human parainfluenza virus type 1, parainfluenza virus type 2, parainfluenza virus type 3, respiratory syncytial virus, viral pneumonia, Chikungunya virus (CHIKV), Venezuelan equine encephalitis (VEEV), dengue (DENV), influenza, West Nile virus (WNV), zika (ZIKV), 229E, NL63, OC43, HKU1, Middle East respiratory syndrome coronavirus (MERS-CoV), severe acute respiratory syndrome coronavirus (SARS-CoV), and severe acute respiratory syndrome coronavirus disease 2019 (SARS-CoV-2), using these compounds. This abstract is intended as a scanning tool for purposes of searching in the particular art and is not intended to be limiting of the present invention.

### CROSS-REFERENCE TO RELATED APPLICATIONS

This application claims the benefit of U.S. Application No. 62/861,837, filed on Jun. 14, 2019, the contents of which are hereby incorporated by reference in their entirety.

### BACKGROUND

Hepatitis B is an infectious disease caused by the hepatitis B virus (HBV) that affects the liver. It can cause both acute and chronic infections. Many people have no symptoms during the initial infection. Some develop a rapid onset of sickness with vomiting, yellowish skin, tiredness, dark urine, and abdominal pain. Often these symptoms last a few weeks and rarely does the initial infection result in death. It may take 30 to 180 days for symptoms to begin. In those who get infected around the time of birth 90% develop chronic hepatitis B while less than 10% of those infected after the age of five do. Most of those with chronic disease have no symptoms; however, cirrhosis and liver cancer may eventually develop. These complications result in the death of 15 to 25% of those with chronic disease.

The virus is transmitted by exposure to infectious blood or body fluids (“Hepatitis B Fact Sheet No. 204,” WHO Int. July 2014). Infection around the time of birth or from contact with other people's blood during childhood is the most frequent method by which hepatitis B is acquired in areas where the disease is common (“Hepatitis B Fact Sheet No. 204,” WHO Int. July 2014). In areas where the disease is rare, intravenous drug use and sexual intercourse are the most frequent routes of infection. Other risk factors include working in healthcare, blood transfusions, dialysis, living with an infected person, travel in countries where the infection rate is high, and living in an institution. Tattooing and acupuncture led to a significant number of cases in the 1980s; however, this has become less common with improved sterility. The hepatitis B viruses cannot be spread by holding hands, sharing eating utensils, kissing, hugging, coughing, sneezing, or breastfeeding. The infection can be diagnosed 30 to 60 days after exposure. The diagnosis is usually confirmed by testing the blood for parts of the virus and for antibodies against the virus ((“Hepatitis B Fact Sheet No. 204,” WHO Int. July 2014). It is one of five main hepatitis viruses: A, B, C, D, and E.

Although therapies for chronic HBV are available, most are limited both in scope and efficacy. Interferon therapy leads to anti-HBs seroconversion in only 3-5% of the patients. Additionally, interferon therapy is very expensive, can have severe side effects, and requires daily injections sub-cutaneously. Newer antiviral agents, such as lamivudine, can reduce viral loads, but lead to anti-HBs seroconversion in only a few patients. Further, they must be used long-term—discontinuation leads to the reappearance of the virus, making the requirement for lifetime treatment a possibility. Thus, there remains a need for a potent therapy that can ameliorate chronic HBV infection remains.

### SUMMARY

In accordance with the purpose(s) of the invention, as embodied and broadly described herein, the invention, in one aspect, relates to compositions and methods for use in the prevention and treatment of viral infections such as, for example, human immunodeficiency virus (HIV), human papillomavirus (HPV), herpes simplex virus (HSV), human cytomegalovirus (HCMV), chicken pox, infectious mononucleosis, mumps, measles, rubella, shingles, ebola, viral gastroenteritis, viral hepatitis, viral meningitis, human metapneumovirus, human parainfluenza virus type 1, parainfluenza virus type 2, parainfluenza virus type 3, respiratory syncytial virus, viral pneumonia, Chikungunya virus (CHIKV), Venezuelan equine encephalitis (VEEV), dengue (DENV), influenza, West Nile virus (WNV), zika (ZIKV), 229E, NL63, OC43, HKU1, Middle East respiratory syndrome coronavirus (MERS-CoV), severe acute respiratory syndrome coronavirus (SARS-CoV), and severe acute respiratory syndrome coronavirus disease 2019 (SARS-CoV-2).

Disclosed are compounds having a structure represented by a formula:

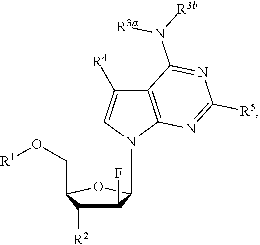

wherein R¹ is selected from hydrogen, —C(O)R¹⁰, —P(O)(OR¹¹)₂, and —P(O)(OR¹¹)R¹²;
wherein R² is selected from hydrogen, —OH, C1-C8 alkoxy, —P(O)(OR^{11′})₂, and —P(O)(OR^{11′})R^{12′}; wherein R¹⁰, when present, is selected from C1-C30 alkyl, C2-C30 alkenyl, and —CH(NH₂)R²⁰; wherein R²⁰, when present, is selected from hydrogen, methyl, isopropyl, isobutyl, sec-butyl, —(CH₂)₃NHC(NH)NH₂, —(CH₂)₄NH₂, —CH₂CO₂H, —(CH₂)₂CO₂H, —CH₂OH, —CH(OH)CH₃, —CH₂C(O)NH₂, —(CH₂)₂C(O)NH₂, —CH₂SH, —(CH₂)₂SCH₃, —CH₂SeH, —CH₂C₆H₅, and —CH₂Cy¹; wherein Cy¹, when present, is selected from monocyclic aryl, para-hydroxy monocyclic aryl, 4-imidazolyl, and 3-indolyl; wherein each of R¹¹ and R^{11′}, when present, is independently selected from hydrogen, C1-C4 alkyl, —(C1-C10 alkyl)CO₂(C1-C10 alkyl), —(C1-C10 alkoxy)CO₂(C1-C10 alkyl), —(C1-C10 alkyl)CO₂(C1-C10 alkylthiol), —(C1-C10 alkyl)-S—S—(C1-C10 alkyl), Ar¹, and —CH₂Ar¹; wherein each occurrence of Ar¹, when present, is selected from aryl and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R¹² and R^{12′}, when present, is selected from —OR²¹ and —NHR²¹; wherein each occurrence of R²¹, when present, is selected from hydrogen, —(C1-C10 alkyl)CO₂(C1-C10 alkyl), —(C1-C10 alkoxy)CO₂(C1-C10 alkyl), —(C1-C10 alkyl)CO₂(C1-C10 alkylthiol), —(C1-C10 alkyl)-S—S—(C1-C10 alkyl), Ar², —CH₂Ar², —P(O)OHOP(O)(OH)₂, and a structure represented by a formula:

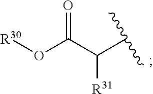

wherein each occurrence of R³⁰, when present, is independently selected from hydrogen, C1-C8 alkyl, Cy², and —CH₂Cy²; wherein each occurrence of Cy², when present, is independently selected from C3-C6 cycloalkyl, aryl, and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each occurrence of R³¹, when present, is independently selected from hydrogen and C1-C8 alkyl; and wherein each occurrence of Ar², when present, is independently selected from aryl and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; or wherein each of R¹ and R² together comprise a structure represented by a formula:

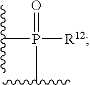

wherein each of R^{3a} and R^{3b} is independently selected from hydrogen, —OH, C1-C10 alkoxy, C1-C8 alkyl, —C(O)(C1-C30 alkyl), —C(O)(C2-C30 alkenyl), Cy³, —CR^{32a}R^{32b}Ar³; wherein each of R^{32a} and R^{32b}, when present, is independently selected from hydrogen and C1-C4 alkyl; wherein Cy³, when present, is C3-C6 cycloalkyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein Ar³, when present, is selected from aryl and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R⁴ is selected from hydrogen, halogen, —CN, —C(O)NH₂, —CO₂H, —COMe, —SO₂Me, C1-C4 haloalkyl, and Ar⁴; wherein Ar⁴, when present, is selected from aryl and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R⁵ is selected from halogen, —CF₃, C1-C10 alkyl, and Ar⁵; and wherein Ar⁵, when present, is selected from aryl and heteroaryl, and is substituted with 0, 1, 2, or 3 groups halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, or a pharmaceutically acceptable salt thereof.

Also disclosed are pharmaceutical compositions comprising a therapeutically effective amount of a disclosed compound and a pharmaceutically acceptable carrier.

Also disclosed are methods of treating a viral infection in a subject, the method comprising the step of administering to the subject an effective amount of a disclosed compound.

Also disclosed are kits comprising a disclosed compound and one or more of: (a) at least one antiviral agent; (b) instructions for administering the compound in connection with treating a viral infection; (c) instructions for administering the compound in connection with reducing the risk of viral infection; and (d) instructions for treating a viral infection.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1A and FIG. 1B show representative images of the antiviral activity of SRI-31416 (FIG. 1A) and 3TC (FIG. 1B) against HBV in HepG2 2.2.15 cells.

FIG. 2A and FIG. 2B show representative images of the antiviral activity of SRI-31416 (FIG. 2A) and acyclovir (FIG. 2B) against HSV-1 Strain HF in Vero cells.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. Definitions

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

As used in the specification and in the claims, the term “comprising” can include the aspects “consisting of” and “consisting essentially of.”

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, “TC₅₀,” is intended to refer to the concentration of a substance (e.g., a compound or a drug) that is required for components of a biological process, including a protein, subunit, organelle, ribonucleoprotein, etc., to grow 50% as well as a control group.

As used herein, “IC₅₀,” is intended to refer to the concentration of a substance (e.g., a compound or a drug) that is required for 50% inhibition of a biological process, or component of a process, including a protein, subunit, organelle, ribonucleoprotein, etc. In one aspect, an IC₅₀ can refer to the concentration of a substance that is required for 50% inhibition in vivo, as further defined elsewhere herein.

As used herein, “EC₅₀,” is intended to refer to the concentration of a substance (e.g., a compound or a drug) that is required for 50% agonism of a biological process, or component of a process, including a protein, subunit, organelle, ribonucleoprotein, etc. In one aspect, an EC₅₀ can refer to the concentration of a substance that is required for 50% agonism in vivo, as further defined elsewhere herein. In a further aspect, EC₅₀ refers to the concentration of agonist that provokes a response halfway between the baseline and maximum response.

As used herein, “EC₅₀,” is intended to refer to the concentration of a substance (e.g., a compound or a drug) that is required for 90% agonism of a biological process, or component of a process, including a protein, subunit, organelle, ribonucleoprotein, etc. In one aspect, an EC₅₀ can refer to the concentration of a substance that is required for 90% agonism in vivo, as further defined elsewhere herein. In a further aspect, EC₅₀ refers to the concentration of agonist that provokes a response 90% above the baseline and 10% below the maximum response.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “dosage form” means a pharmacologically active material in a medium, carrier, vehicle, or device suitable for administration to a subject. A dosage forms can comprise inventive a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene 9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form, which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64^{th} edition), and The Pharmacological Basis of Therapeutics (12^{th}, edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, and amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions that can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by the formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A4) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized 71 electrons above and below the plane of the molecule, where the 71 clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl can be two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen,” or “halide,” as used herein can be used interchangeably and refer to F, Cl, Br, or I.

The terms “pseudohalide,” “pseudohalogen,” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl,” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl,” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl”, “heteroaryl”, “bicyclic heterocycle” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl,” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxyl” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula -SiA¹A2A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogen of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{◯}; —(CH₂)₀₋₄OR^{◯}; —O(CH₂)₀₋₄R^{◯}, —O—(CH₂)₀₋₄C(O)OR^{◯}; —(CH₂)₀₋₄CH(OR^{◯})₂; —(CH₂)₀₋₄SR^{◯}; —(CH₂)₀₋₄Ph, which may be substituted with R^{◯}; —(CH₂)₀₋₄O(CH₂)₀₋₁Ph which may be substituted with R^{◯}; —CH═CHPh, which may be substituted with R^{◯}; —(CH₂)₀₋₄O(CH₂)₀₋₁-pyridyl which may be substituted with R^{◯}; —NO₂; —CN; —N₃; —(CH₂)₀₋₄N(R^{◯})₂; —(CH₂)₀₋₄N(R^{◯})C(O)R^{◯}; —N(R^{◯})C(S)R^{◯}; —(CH₂)₀₋₄N(R^{◯})C(O)NR^{◯}₂; —N(R^{◯})C(S)NR^{◯}₂; —(CH₂)₀₋₄N(R^{◯})C(O)OR^{◯}; —N(R^{◯})N(R^{◯})C(O)R^{◯}; —N(R^{◯})N(R^{◯})C(O)NR^{◯}₂; —N(R^{◯})N(R^{◯})C(O)OR^{◯}; —(CH₂)₀₋₄C(O)R^{◯}; —C(S)R^{◯}; —(CH₂)₀₋₄C(O)OR^{◯}; —(CH₂)₀₋₄C(O)SR^{◯}; —(CH₂)₀₋₄C(O)OSiR^{◯}₃; —(CH₂)₀₋₄OC(O)R^{◯}; —OC(O)(CH₂)₀₋₄SR—, SC(S)SR^{◯}; —(CH₂)₀₋₄SC(O)R^{◯}; —(CH₂)₀₋₄C(O)NR^{◯}₂; —C(S)NR^{◯}₂; —C(S)SR^{◯}; —(CH₂)₀₋₄OC(O)NR^{◯}₂; —C(O)N(OR^{◯})R^{◯}; —C(O)C(O)R^{◯}; —C(O)CH₂C(O)R^{◯}; —C(NOR^{◯})R^{◯}; —(CH₂)₀₋₄SSR^{◯}; —(CH₂)₀₋₄S(O)₂R^{◯}; —(CH₂)₀₋₄S(O)₂OR^{◯}; —(CH₂)₀₋₄OS(O)₂R^{◯}; —S(O)₂NR^{◯}₂; —(CH₂)₀₋₄S(O)R^{◯}; —N(R^{◯})S(O)₂NR^{◯}₂; —N(R^{◯})S(O)₂R^{◯}; —N(OR^{◯})R^{◯}; —C(NH)NR^{◯}₂; —P(O)₂R^{◯}; —P(O)R^{◯}₂; —OP(O)R^{◯}₂; —OP(O)(OR^{◯})₂; SiR^{◯}₃; —(C₁₋₄ straight or branched alkylene)O—N(R^{◯})₂; or —(C₁₋₄ straight or branched alkylene)C(O)O—N(R^{◯})₂, wherein each R^{◯} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{◯}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{◯} (or the ring formed by taking two independent occurrences of R^{◯} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{◯} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon-containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

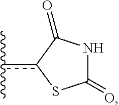

regardless of whether thiazolidinedione is use to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5,6,7,8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

When the disclosed compounds contain one chiral center, the compounds exist in two enantiomeric forms. Unless specifically stated to the contrary, a disclosed compound includes both enantiomers and mixtures of enantiomers, such as the specific 50:50 mixture referred to as a racemic mixture. The enantiomers can be resolved by methods known to those skilled in the art, such as formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step can liberate the desired enantiomeric form. Alternatively, specific enantiomers can be synthesized by asymmetric synthesis using optically active reagents, substrates, catalysts or solvents, or by converting one enantiomer into the other by asymmetric transformation.

Designation of a specific absolute configuration at a chiral carbon in a disclosed compound is understood to mean that the designated enantiomeric form of the compounds can be provided in enantiomeric excess (e.e.). Enantiomeric excess, as used herein, is the presence of a particular enantiomer at greater than 50%, for example, greater than 60%, greater than 70%, greater than 75%, greater than 80%, greater than 85%, greater than 90%, greater than 95%, greater than 98%, or greater than 99%. In one aspect, the designated enantiomer is substantially free from the other enantiomer. For example, the “R” forms of the compounds can be substantially free from the “S” forms of the compounds and are, thus, in enantiomeric excess of the “S” forms. Conversely, “S” forms of the compounds can be substantially free of “R” forms of the compounds and are, thus, in enantiomeric excess of the “R” forms.

When a disclosed compound has two or more chiral carbons, it can have more than two optical isomers and can exist in diastereoisomeric forms. For example, when there are two chiral carbons, the compound can have up to four optical isomers and two pairs of enantiomers ((S,S)/(R,R) and (R,S)/(S,R)). The pairs of enantiomers (e.g., (S,S)/(R,R)) are mirror image stereoisomers of one another. The stereoisomers that are not mirror-images (e.g., (S,S) and (R,S)) are diastereomers. The diastereoisomeric pairs can be separated by methods known to those skilled in the art, for example chromatography or crystallization and the individual enantiomers within each pair may be separated as described above. Unless otherwise specifically excluded, a disclosed compound includes each diastereoisomer of such compounds and mixtures thereof.

The compounds according to this disclosure may form prodrugs at hydroxyl or amino functionalities using alkoxy, amino acids, etc., groups as the prodrug forming moieties. For instance, the hydroxymethyl position may form mono-, di- or triphosphates and again these phosphates can form prodrugs. Preparations of such prodrug derivatives are discussed in various literature sources (examples are: Alexander et al., J. Med. Chem. 1988, 31, 318; Aligas-Martin et al., PCT WO 2000/041531, p. 30). The nitrogen function converted in preparing these derivatives is one (or more) of the nitrogen atoms of a compound of the disclosure.

“Derivatives” of the compounds disclosed herein are pharmaceutically acceptable salts, prodrugs, deuterated forms, radioactively labeled forms, isomers, solvates and combinations thereof. The “combinations” mentioned in this context are refer to derivatives falling within at least two of the groups: pharmaceutically acceptable salts, prodrugs, deuterated forms, radioactively labeled forms, isomers, and solvates. Examples of radioactively labeled forms include compounds labeled with tritium, phosphorous-32, iodine-129, carbon-11, fluorine-18, and the like.

Compounds described herein comprise atoms in both their natural isotopic abundance and in non-natural abundance. The disclosed compounds can be isotopically labeled or isotopically substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically labeled compounds of the present invention, for example those into which radioactive isotopes such as ³H and ¹⁴C are incorporated, are useful in drug and/or substrate tissue distribution assays. Tritiated, i.e., 3H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

The term “co-crystal” means a physical association of two or more molecules that owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an α-hydrogen can exist in an equilibrium of the keto form and the enol form.

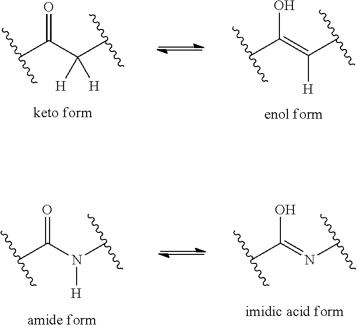 [SMILES: *CC(*)=O ; */C=C(/*)O ; *NC(*)=O ; */N=C(/*)O]

Likewise, amides with an N-hydrogen can exist in an equilibrium of the amide form and the imidic acid form. As another example, pyrazoles can exist in two tautomeric forms, N¹-unsubstituted, 3-A³ and N¹-unsubstituted, 5-A³ as shown below.

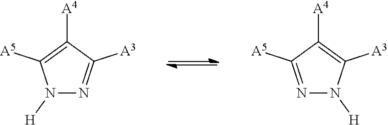

Unless stated to the contrary, the invention includes all such possible tautomers.

It is known that chemical substances form solids that are present in different states of order that are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

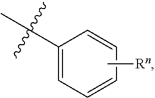

which is understood to be equivalent to a formula:

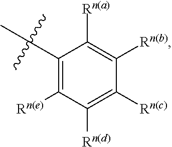

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance Rⁿ(a) is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Strem Chemicals (Newburyport, Mass.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and supplemental volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. Compounds

In one aspect, the invention relates to compounds useful in treating disorders associated with a viral infection, in particular, human immunodeficiency virus (HIV), human papillomavirus (HPV), herpes simplex virus (HSV), human cytomegalovirus (HCMV), chicken pox, infectious mononucleosis, mumps, measles, rubella, shingles, ebola, viral gastroenteritis, viral hepatitis, viral meningitis, human metapneumovirus, human parainfluenza virus type 1, parainfluenza virus type 2, parainfluenza virus type 3, respiratory syncytial virus, viral pneumonia, Chikungunya virus (CHIKV), Venezuelan equine encephalitis (VEEV), dengue (DENV), influenza, West Nile virus (WNV), and zika (ZIKV). In a further aspect, the disorder is viral hepatitis.

In one aspect, the disclosed compounds exhibit antiviral activity.

In one aspect, the compounds of the invention are useful in inhibiting viral activity in a mammal. In a further aspect, the compounds of the invention are useful in inhibiting viral activity in at least one cell.

In one aspect, the compounds of the invention are useful in the treatment of viral infections, as further described herein.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

### 1. Structure

In one aspect, disclosed are compounds having a structure represented by a formula:

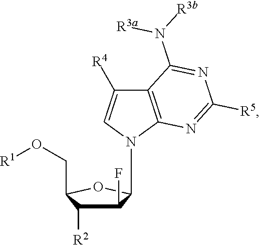

wherein R¹ is selected from hydrogen, —C(O)R¹⁰, —P(O)(OR¹¹)₂, and —P(O)(OR¹¹)R¹²; wherein R² is selected from hydrogen, —OH, C1-C8 alkoxy, —P(O)(OR^{11′})₂, and —P(O)(OR^{11′})R^{12′}; wherein R¹⁰, when present, is selected from C1-C30 alkyl, C2-C30 alkenyl, and —CH(NH₂)R²⁰; wherein R²⁰, when present, is selected from hydrogen, methyl, isopropyl, isobutyl, sec-butyl, —(CH₂)₃NHC(NH)NH₂, —(CH₂)₄NH₂, —CH₂CO₂H, —(CH₂)₂CO₂H, —CH₂OH, —CH(OH)CH₃, —CH₂C(O)NH₂, —(CH₂)₂C(O)NH₂, —CH₂SH, —(CH₂)₂SCH₃, —CH₂SeH, —CH₂C₆H₅, and —CH₂Cy¹; wherein Cy¹, when present, is selected from monocyclic aryl, para-hydroxy monocyclic aryl, 4-imidazolyl, and 3-indolyl; wherein each of R¹¹ and R^{11′}, when present, is independently selected from hydrogen, C1-C4 alkyl, —(C1-C10 alkyl)CO₂(C1-C10 alkyl), —(C1-C10 alkoxy)CO₂(C1-C10 alkyl), —(C1-C10 alkyl)CO₂(C1-C10 alkylthiol), —(C1-C10 alkyl)-S—S—(C1-C10 alkyl), Ar¹, and —CH₂Ar¹; wherein each occurrence of Ar¹, when present, is selected from aryl and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R¹² and R^{12′}, when present, is selected from —OR²¹ and —NHR²¹; wherein each occurrence of R²¹, when present, is selected from hydrogen, —(C1-C10 alkyl)CO₂(C1-C10 alkyl), —(C1-C10 alkoxy)CO₂(C1-C10 alkyl), —(C1-C10 alkyl)CO₂(C1-C10 alkylthiol), —(C1-C10 alkyl)-S—S—(C1-C10 alkyl), Ar², —CH₂Ar², —P(O)OHOP(O)(OH)₂, and a structure represented by a formula:

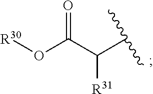

wherein each occurrence of R³⁰, when present, is independently selected from hydrogen, C1-C8 alkyl, Cy², and —CH₂Cy²; wherein each occurrence of Cy², when present, is independently selected from C3-C6 cycloalkyl, aryl, and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each occurrence of R³¹, when present, is independently selected from hydrogen and C1-C8 alkyl; and wherein each occurrence of Ar², when present, is independently selected from aryl and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; or wherein each of R¹ and R² together comprise a structure represented by a formula:

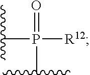

wherein each of R^{3a} and R^{3b} is independently selected from hydrogen, —OH, C1-C10 alkoxy, C1-C8 alkyl, —C(O)(C1-C30 alkyl), —C(O)(C2-C30 alkenyl), Cy³, —CR^{32a}R^{32b}Ar³; wherein each of R^{32a} and R^{32b}, when present, is independently selected from hydrogen and C1-C4 alkyl; wherein Cy³, when present, is C3-C6 cycloalkyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein Ar³, when present, is selected from aryl and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R⁴ is selected from hydrogen, halogen, —CN, —C(O)NH₂, —CO₂H, —COMe, —SO₂Me, C1-C4 haloalkyl, and Ar⁴; wherein Ar⁴, when present, is selected from aryl and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R⁵ is selected from halogen, —CF₃, C1-C10 alkyl, and Ar⁵; and wherein Ar⁵, when present, is selected from aryl and heteroaryl, and is substituted with 0, 1, 2, or 3 groups halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, or a pharmaceutically acceptable salt thereof.

In a further aspect, the compound has a structure represented by a formula:

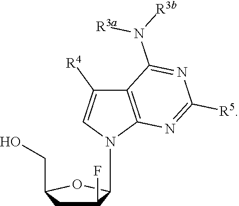

In a further aspect, the compound has a structure represented by a formula:

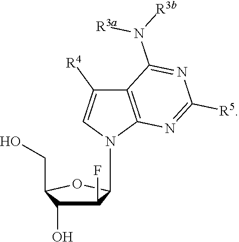

In a further aspect, the compound has a structure represented by a formula:

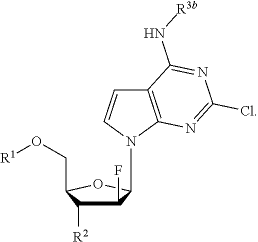

In a further aspect, the compound has a structure represented by a formula:

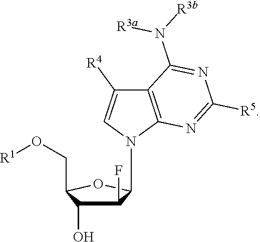

In a further aspect, the compound has a structure represented by a formula:

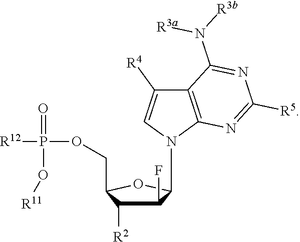

In a further aspect, the compound has a structure represented by a formula:

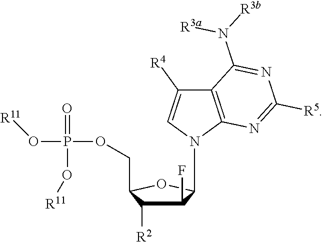

In a further aspect, the compound has a structure represented by a formula:

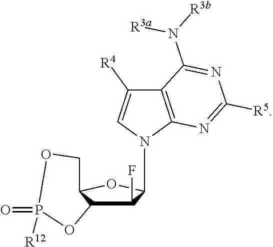

In a further aspect, the compound has a structure represented by a formula:

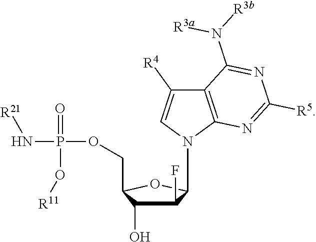

In a further aspect, the compound has a structure represented by a formula:

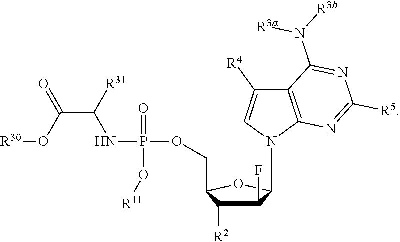

In a further aspect, the compound has a structure represented by a formula:

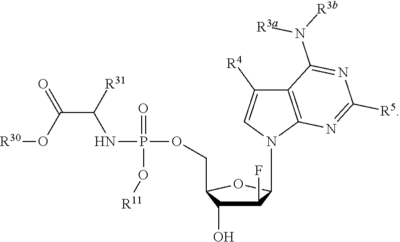

In a further aspect, the compound has a structure represented by a formula:

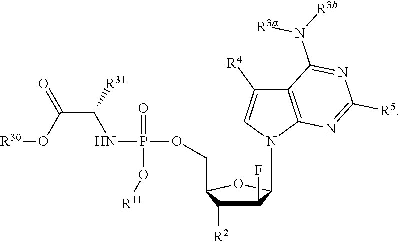

In a further aspect, the compound has a structure represented by a formula:

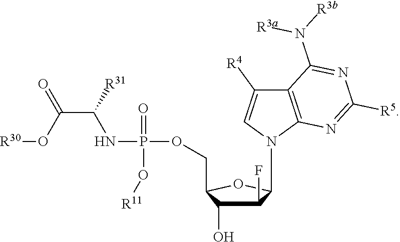

In a further aspect, the compound is selected from:

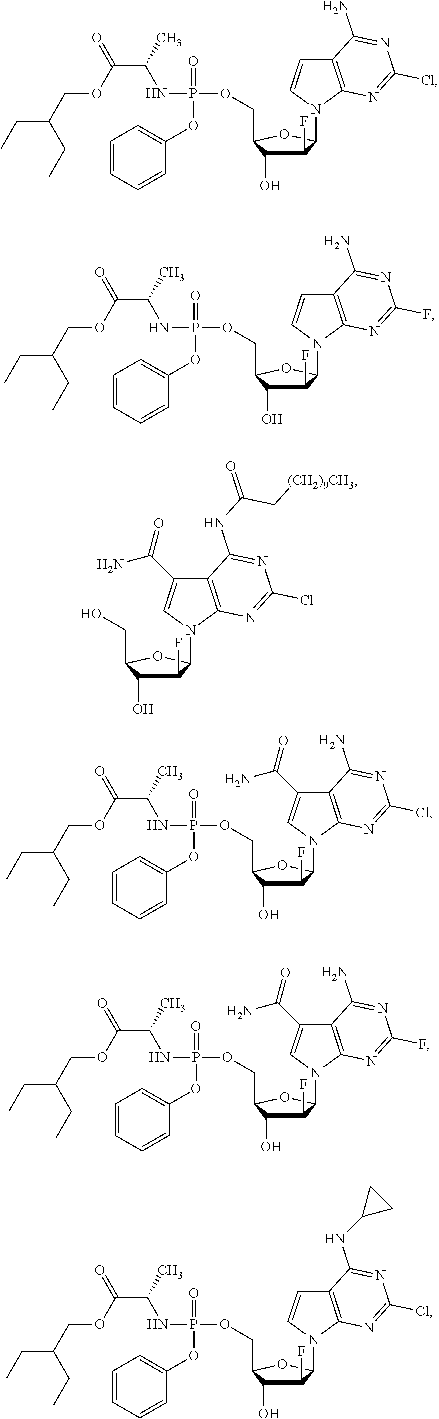

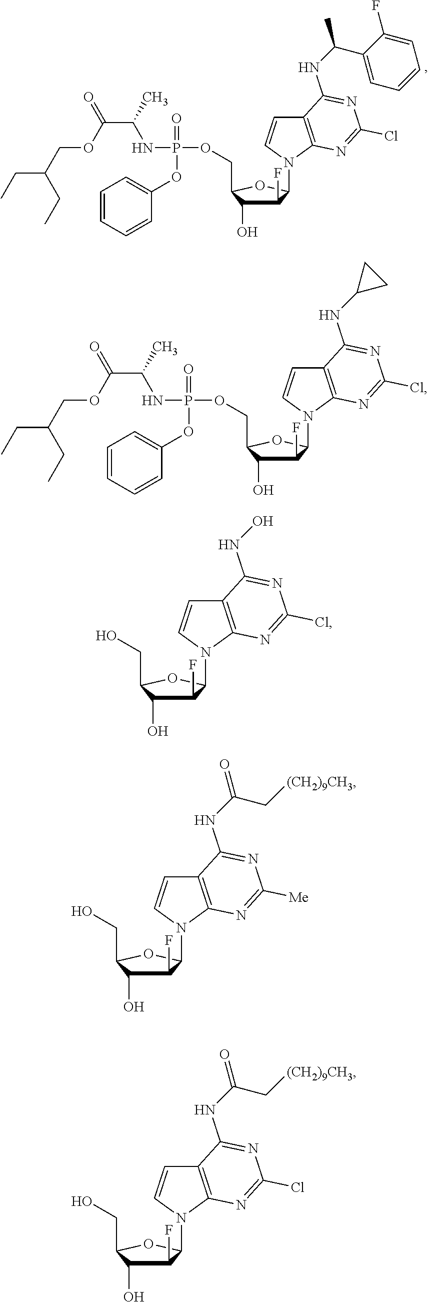

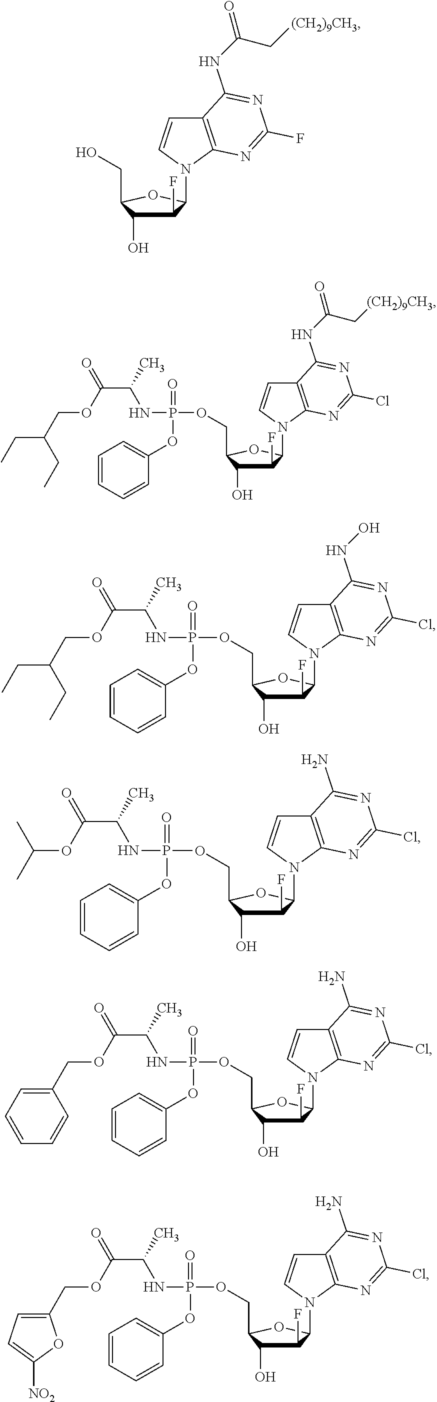

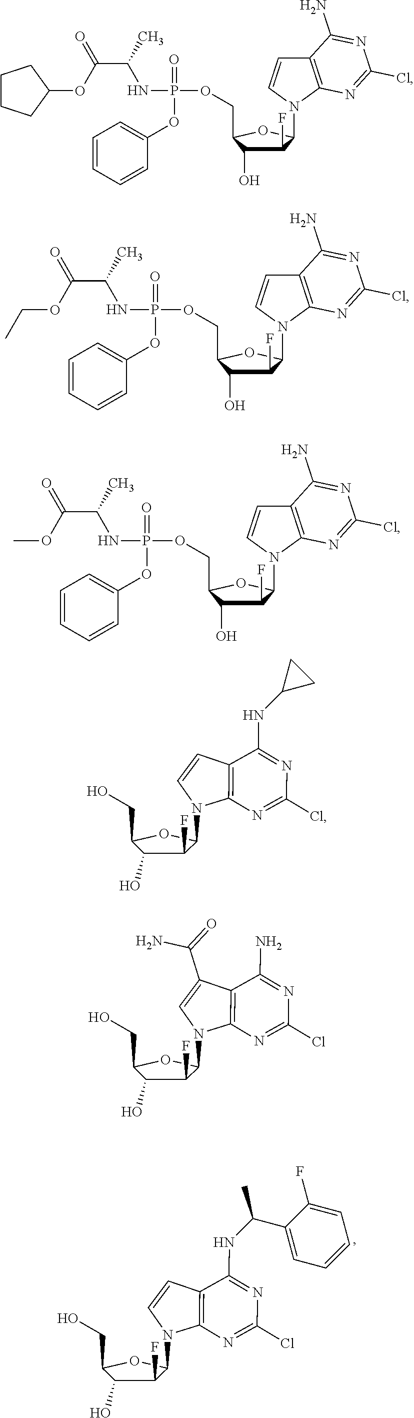

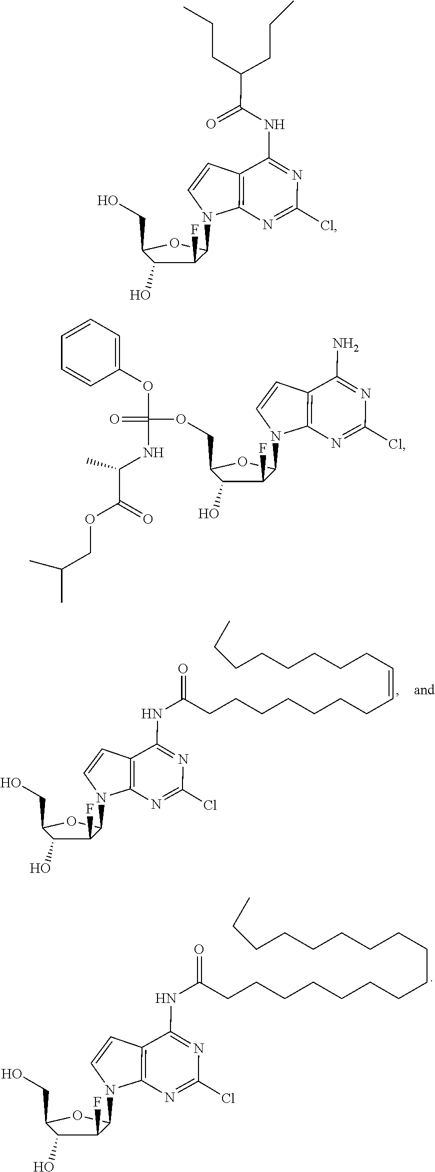

a. R¹ and R² Groups

In one aspect, R¹ is selected from hydrogen, —C(O)R¹⁰, —P(O)(OR¹¹)₂, and —P(O)(OR¹¹)R¹² and R² is selected from hydrogen, —OH, C1-C8 alkoxy, —P(O)(OR¹¹)₂, and —P(O)(OR^{11′})R^{12′}, or each of R¹ and R² together comprise a structure represented by a formula:

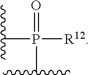

In one aspect, R¹ is selected from hydrogen, —C(O)R¹⁰, —P(O)(OR¹¹)₂, and —P(O)(OR¹¹)R¹². In a further aspect, R¹ is selected from hydrogen, —P(O)(OR¹¹)₂, and —P(O)(OR¹¹)R¹². In a still further aspect, R¹ is selected from hydrogen and —P(O)(OR¹¹)R¹². In yet a further aspect, R¹ is selected from hydrogen and —P(O)(OR¹¹)₂.

In one aspect, R² is selected from hydrogen, —OH, C1-C8 alkoxy, —P(O)(OR^{11′})₂, and —P(O)(OR^{11′})R^{12′}. In a further aspect, R² is selected from hydrogen, —OH, C1-C4 alkoxy, —P(O)(OR^{11′})₂, and —P(O)(OR^{11′})R^{12′}. In a still further aspect, R² is selected from hydrogen, —OH, methoxy, ethoxy, n-propoxy, isopropoxy, —P(O)(OR^{11′})₂, and —P(O)(OR^{11′})R^{12′}. In yet a further aspect, R² is selected from hydrogen, —OH, methoxy, ethoxy, —P(O)(OR^{11′})₂, and —P(O)(OR^{11′})R^{12′}. In an even further aspect, R² is selected from hydrogen, —OH, methoxy, —P(O)(OR^{11′})₂, and —P(O)(OR^{11′})R^{12′}.

In one aspect, each of R¹ and R² together comprise a structure represented by a formula:

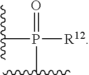

In a further aspect, R¹ is hydrogen.

In a further aspect, R¹ is selected from hydrogen and —C(O)R¹⁰. In a still further aspect, R¹ is —C(O)R¹⁰.

In a further aspect, R¹ is selected from —P(O)(OR¹¹)₂ and —P(O)(OR¹¹)R¹². In a still further aspect, R¹ is —P(O)(OR¹¹)₂. In yet a further aspect, R¹ is —P(O)(OR¹¹)R¹².

In a further aspect, R² is selected from hydrogen and —OH. In a still further aspect, R² is —OH. In yet a further aspect, R² is hydrogen.

In a further aspect, R² is selected from hydrogen, —OH, and C1-C8 alkoxy. In a still further aspect, R² is selected from hydrogen, —OH, and C1-C4 alkoxy. In yet a further aspect, R² is selected from hydrogen, —OH, methoxy, ethoxy, n-propoxy, and isopropoxy. In an even further aspect, R² is selected from hydrogen, —OH, methoxy, and ethoxy. In a still further aspect, R² is selected from hydrogen, —OH, and methoxy.

In a further aspect, R² is selected from hydrogen and C1-C8 alkoxy. In a still further aspect, R² is selected from hydrogen and C1-C4 alkoxy. In yet a further aspect, R² is selected from hydrogen, methoxy, ethoxy, n-propoxy, and isopropoxy. In an even further aspect, R² is selected from hydrogen, methoxy, and ethoxy. In a still further aspect, R² is selected from hydrogen and ethoxy. In yet a further aspect, R² is selected from hydrogen and methoxy.

In a further aspect, R² is C1-C8 alkoxy. In a still further aspect, R² is C1-C4 alkoxy. In yet a further aspect, R² is selected from methoxy, ethoxy, n-propoxy, and isopropoxy. In an even further aspect, R² is selected from methoxy and ethoxy. In a still further aspect, R² is ethoxy. In yet a further aspect, R² is methoxy.

In a further aspect, R² is selected from hydrogen, —P(O)(OR^{11′})₂, and —P(O)(OR^{11′})R^{12′}. In a still further aspect, R² is selected from hydrogen and —P(O)(OR^{11′})₂. In yet a further aspect, R² is selected from hydrogen and —P(O)(OR^{11′})R^{12′}.

In a further aspect, R² is selected from —P(O)(OR^{11′})₂ and —P(O)(OR^{11′})R^{12′} In a still further aspect, R² is —P(O)(OR^{11′})₂. In yet a further aspect, R² is —P(O)(OR^{11′})R^{12′}.

b. R^{3a} and R^{3b} Groups

In one aspect, each of R^{3a} and R^{3b} is independently selected from hydrogen, —OH, C1-C10 alkoxy, C1-C8 alkyl, —C(O)(C1-C30 alkyl), —C(O)(C2-C30 alkenyl), Cy³, and —CR^{32a}R^{32b}Ar³. In a further aspect, each of R^{3a} and R^{3b} is independently selected from hydrogen, —OH, C1-C10 alkoxy, C1-C8 alkyl, —C(O)(C1-C15 alkyl), —C(O)(C2-C15 alkenyl), Cy³, and —CR^{32a}R^{32b}Ar³. In a still further aspect, each of R^{3a} and R^{3b} is independently selected from hydrogen, —OH, C1-C10 alkoxy, C1-C8 alkyl, —C(O)(C1-C10 alkyl), —C(O)(C2-C10 alkenyl), Cy³, and —CR^{32a}R^{32b}Ar³. In yet a further aspect, each of R^{3a} and R^{3b} is independently selected from hydrogen, —OH, C1-C8 alkoxy, C1-C8 alkyl, —C(O)(C1-C8 alkyl), —C(O)(C2-C8 alkenyl), Cy³, and —CR^{32a}R^{32b}Ar³. In an even further aspect, each of R^{3a} and R^{3b} is independently selected from hydrogen, —OH, C1-C4 alkoxy, C1-C4 alkyl, —C(O)(C1-C4 alkyl), —C(O)(C2-C4 alkenyl), Cy³, and —CR^{32a}R^{32b}Ar³. In a still further aspect, each of R^{3a} and R^{3b} is independently selected from hydrogen, —OH, methoxy, ethoxy, n-propoxy, isopropoxy, methyl, ethyl, n-propyl, isopropyl, —C(O)CH₃, —C(O)CH₂CH₃, —C(O)CH₂CH₂CH₃, —C(O)CH(CH₃)₂, —C(O)CH═CH₂, —C(O)CH₂CH═CH₂, —C(O)CH═CH₂CH₃, —C(O)C(CH₃)═CH₂, Cy³, and —CR^{32a}R^{32b}Ar³. In yet a further aspect, each of R^{3a} and R^{3b} is independently selected from hydrogen, —OH, methoxy, ethoxy, methyl, ethyl, —C(O)CH₃, —C(O)CH₂CH₃, —C(O)CH═CH₂, Cy³, and —CR^{32a}R^{32b}Ar³. In an even further aspect, each of R^{3a} and R^{3b} is independently selected from hydrogen, —OH, methoxy, methyl, —C(O)CH₃, Cy³, and —CR^{32a}R^{32b}Ar³.

In a further aspect, each of R^{3a} and R^{3b} is independently selected from hydrogen and —OH. In a still further aspect, each of R^{3a} and R^{3b} is —OH. In yet a further aspect, each of R^{3a} and R^{3b} is hydrogen.

In various aspects, each of R^{3a} and R^{3b} is independently selected from hydrogen, —OH, C1-C10 alkoxy, C1-C8 alkyl, Cy³, and —CR^{32a}R^{32b}Ar³. In a further aspect, each of R^{3a} and R^{3b} is independently selected from hydrogen, —OH, C1-C8 alkoxy, C1-C8 alkyl, Cy³, and —CR^{32a}R^{32b}Ar³. In an even further aspect, each of R^{3a} and R^{3b} is independently selected from hydrogen, —OH, C1-C4 alkoxy, C1-C4 alkyl, Cy³, and —CR^{32a}R^{32b}Ar³. In a still further aspect, each of R^{3a} and R^{3b} is independently selected from hydrogen, —OH, methoxy, ethoxy, n-propoxy, isopropoxy, methyl, ethyl, n-propyl, isopropyl, Cy³, and —CR^{32a}R^{32b}Ar³. In yet a further aspect, each of R^{3a} and R^{3b} is independently selected from hydrogen, —OH, methoxy, ethoxy, methyl, ethyl, Cy³, and —CR^{32a}R^{32b}Ar³. In an even further aspect, each of R^{3a} and R^{3b} is independently selected from hydrogen, —OH, methoxy, methyl, Cy³, and —CR^{32a}R^{32b}Ar³.

In various aspects, each of R^{3a} and R^{3b} is independently selected from hydrogen, C1-C10 alkoxy, and C1-C8 alkyl. In a further aspect, each of R^{3a} and R^{3b} is independently selected from hydrogen, C1-C8 alkoxy, and C1-C8 alkyl. In an even further aspect, each of R^{3a} and R^{3b} is independently selected from hydrogen, C1-C4 alkoxy, and C1-C4 alkyl. In a still further aspect, each of R^{3a} and R^{3b} is independently selected from hydrogen, methoxy, ethoxy, n-propoxy, isopropoxy, methyl, ethyl, n-propyl, and isopropyl. In yet a further aspect, each of R^{3a} and R^{3b} is independently selected from hydrogen, methoxy, ethoxy, methyl, and ethyl. In an even further aspect, each of R^{3a} and R^{3b} is independently selected from hydrogen, methoxy, and methyl.

In various aspects, each of R^{3a} and R^{3b} is independently selected from hydrogen and C1-C8 alkyl. In a further aspect, each of R^{3a} and R^{3b} is independently selected from hydrogen and C1-C4 alkyl. In a still further aspect, each of R^{3a} and R^{3b} is independently selected from hydrogen, methyl, ethyl, n-propyl, and isopropyl. In yet a further aspect, each of R^{3a} and R^{3b} is independently selected from hydrogen, methyl, and ethyl. In an even further aspect, each of R^{3a} and R^{3b} is independently selected from hydrogen and methyl.

In various aspects, each of R^{3a} and R^{3b} is independently selected from hydrogen and C1-C10 alkoxy. In a further aspect, each of R^{3a} and R^{3b} is independently selected from hydrogen and C1-C8 alkoxy. In an even further aspect, each of R^{3a} and R^{3b} is independently selected from hydrogen and C1-C4 alkoxy. In a still further aspect, each of R^{3a} and R^{3b} is independently selected from hydrogen, methoxy, ethoxy, n-propoxy, and isopropoxy. In yet a further aspect, each of R^{3a} and R^{3b} is independently selected from hydrogen, methoxy, and ethoxy. In an even further aspect, each of R^{3a} and R^{3b} is independently selected from hydrogen and methoxy.

In a further aspect, each of R^{3a} and R^{3b} is independently selected from hydrogen, Cy³, and —CR^{32a}R^{32b}Ar³. In a still further aspect, each of R^{3a} and R^{3b} is independently selected from hydrogen and Cy³. In yet a further aspect, each of R^{3a} and R^{3b} is independently selected from hydrogen and —CR^{32a}R^{32b}Ar³.

In various aspects, each of R^{3a} and R^{3b} is independently selected from hydrogen, —OH, —C(O)(C1-C30 alkyl), —C(O)(C2-C30 alkenyl), Cy³, and —CR^{32a}R^{32b}Ar³. In a further aspect, each of R^{3a} and R^{3b} is independently selected from hydrogen, —OH, —C(O)(C1-C15 alkyl), —C(O)(C2-C15 alkenyl), Cy³, and —CR^{32a}R^{32b}Ar³. In a still further aspect, each of R^{3a} and R^{3b} is independently selected from hydrogen, —OH, —C(O)(C1-C10 alkyl), —C(O)(C2-C10 alkenyl), Cy³, and —CR^{32a}R^{32b}Ar³. In yet a further aspect, each of R^{3a} and R^{3b} is independently selected from hydrogen, —OH, —C(O)(C1-C8 alkyl), —C(O)(C2-C8 alkenyl), Cy³, and —CR^{32a}R^{32b}Ar³. In an even further aspect, each of R^{3a} and R^{3b} is independently selected from hydrogen, —OH, —C(O)(C1-C4 alkyl), —C(O)(C2-C4 alkenyl), Cy³, and —CR^{32a}R^{32b}Ar³. In a still further aspect, each of R^{3a} and R^{3b} is independently selected from hydrogen, —OH, —C(O)CH₃, —C(O)CH₂CH₃, —C(O)CH₂CH₂CH₃, —C(O)CH(CH₃)₂, —C(O)CH═CH₂, —C(O)CH₂CH═CH₂, —C(O)CH═CH₂CH₃, —C(O)C(CH₃)═CH₂, Cy³, and —CR^{32a}R^{32b}Ar³. In yet a further aspect, each of R^{3a} and R^{3b} is independently selected from hydrogen, —OH, —C(O)CH₃, —C(O)CH₂CH₃, —C(O)CH═CH₂, Cy³, and —CR^{32a}R^{32b}Ar³. In an even further aspect, each of R^{3a} and R^{3b} is independently selected from hydrogen, —OH, —C(O)CH₃, Cy³, and —CR^{32a}R^{32b}Ar³.

In various aspects, each of R^{3a} and R^{3b} is independently selected from hydrogen, —C(O)(C1-C30 alkyl), and —C(O)(C2-C30 alkenyl). In a further aspect, each of R^{3a} and R^{3b} is independently selected from hydrogen, —C(O)(C1-C15 alkyl), and —C(O)(C2-C15 alkenyl). In a still further aspect, each of R^{3a} and R^{3b} is independently selected from hydrogen, —C(O)(C1-C10 alkyl), and —C(O)(C2-C10 alkenyl). In yet a further aspect, each of R^{3a} and R^{3b} is independently selected from hydrogen, —C(O)(C1-C8 alkyl), and —C(O)(C2-C8 alkenyl). In an even further aspect, each of R^{3a} and R^{3b} is independently selected from hydrogen, —C(O)(C1-C4 alkyl), and —C(O)(C2-C4 alkenyl). In a still further aspect, each of R^{3a} and R^{3b} is independently selected from hydrogen, —C(O)CH₃, —C(O)CH₂CH₃, —C(O)CH₂CH₂CH₃, —C(O)CH(CH₃)₂, —C(O)CH═CH₂, —C(O)CH₂CH═CH₂, —C(O)CH═CH₂CH₃, and —C(O)C(CH₃)═CH₂. In yet a further aspect, each of R^{3a} and R^{3b} is independently selected from hydrogen, —C(O)CH₃, —C(O)CH₂CH₃, and —C(O)CH═CH₂. In an even further aspect, each of R^{3a} and R^{3b} is independently selected from hydrogen and —C(O)CH₃.

c. R⁴ Groups

In one aspect, R⁴ is selected from hydrogen, halogen, —CN, —C(O)NH₂, —CO₂H, —COMe, —SO₂Me, C1-C4 haloalkyl, and Ar⁴. In a further aspect, R⁴ is selected from hydrogen, —F, —Cl, —Br, —CN, —C(O)NH₂, —CO₂H, —COMe, —SO₂Me, —CF₃, —CHF₂, —CH₂F, —CH₂CH₂F, —CH₂CH₂CH₂F, —CH(CH₃)CH₂F, and Ar⁴. In a still further aspect, R⁴ is selected from hydrogen, —F, —Cl, —Br, —CN, —C(O)NH₂, —CO₂H, —COMe, —SO₂Me, —CF₃, —CHF₂, —CH₂F, —CH₂CH₂F, and Ar⁴. In yet a further aspect, R⁴ is selected from hydrogen, —F, —Cl, —Br, —CN, —C(O)NH₂, —CO₂H, —COMe, —SO₂Me, —CF₃, —CHF₂, —CH₂F, and Ar⁴.

In a further aspect, R⁴ is selected from hydrogen and —CN. In a still further aspect, R⁴ is —CN. In yet a further aspect, R⁴ is hydrogen.

In various aspects, R⁴ is selected from hydrogen, —CN, —C(O)NH₂, —CO₂H, —COMe, —SO₂Me, and Ar⁴. In a further aspect, R⁴ is selected from hydrogen, —C(O)NH₂, —CO₂H, —COMe, —SO₂Me, and Ar⁴. In a still further aspect, R⁴ is selected from hydrogen, —C(O)NH₂, —CO₂H, —COMe, and Ar⁴. In yet a further aspect, R⁴ is selected from hydrogen, —C(O)NH₂, —CO₂H, and —COMe. In an even further aspect, R⁴ is selected from hydrogen, —C(O)NH₂, and —CO₂H. In a still further aspect, R⁴ is selected from hydrogen and —C(O)NH₂. In yet a further aspect, R⁴ is selected from hydrogen and —CO₂H. In an even further aspect, R⁴ is —C(O)NH₂. In a still further aspect, R⁴ is —CO₂H.

In a further aspect, R⁴ is selected from hydrogen, —SO₂Me, and Ar⁴. In a still further aspect, R⁴ is selected from hydrogen and —SO₂Me. In yet a further aspect, R⁴ is selected from hydrogen and Ar⁴. In an even further aspect, R⁴ is —SO₂Me. In a still further aspect, R⁴ is Ar⁴.

In a further aspect, R⁴ is selected from hydrogen and —COMe. In a still further aspect, R⁴ is —COMe.

In various aspects, R⁴ is selected from hydrogen, halogen, and C1-C4 haloalkyl. In a further aspect, R⁴ is selected from hydrogen, —F, —Cl, —Br, —CF₃, —CHF₂, —CH₂F, —CH₂CH₂F, —CH₂CH₂CH₂F, and —CH(CH₃)CH₂F. In a still further aspect, R⁴ is selected from hydrogen, —F, —Cl, —CF₃, —CHF₂, —CH₂F, and —CH₂CH₂F. In yet a further aspect, R⁴ is selected from hydrogen, —F, —CF₃, —CHF₂, and —CH₂F.

In various aspects, R⁴ is selected from hydrogen and halogen. In a further aspect, R⁴ is selected from hydrogen, —F, —Cl, and —Br. In a still further aspect, R⁴ is selected from hydrogen, —F, and —Cl. In yet a further aspect, R⁴ is selected from hydrogen and —F.

In various aspects, R⁴ is halogen. In a further aspect, R⁴ is selected from —F, —Cl, and —Br. In a still further aspect, R⁴ is selected from —F and —Cl. In yet a further aspect, R⁴ is —F.

In various aspects, R⁴ is selected from hydrogen and C1-C4 haloalkyl. In a further aspect, R⁴ is selected from hydrogen, —CF₃, —CHF₂, —CH₂F, —CH₂CH₂F, —CH₂CH₂CH₂F, and —CH(CH₃)CH₂F. In a still further aspect, R⁴ is selected from hydrogen, —CF₃, —CHF₂, —CH₂F, and —CH₂CH₂F. In yet a further aspect, R⁴ is selected from hydrogen, —CF₃, —CHF₂, and —CH₂F.

In various aspects, R⁴ is C1-C4 haloalkyl. In a further aspect, R⁴ is selected from —CF₃, —CHF₂, —CH₂F, —CH₂CH₂F, —CH₂CH₂CH₂F, and —CH(CH₃)CH₂F. In a still further aspect, R⁴ is selected from —CF₃, —CHF₂, —CH₂F, and —CH₂CH₂F. In yet a further aspect, R⁴ is selected from —CF₃, —CHF₂, and —CH₂F.

d. R⁵ Groups

In one aspect, R⁵ is selected from halogen, —CF₃, C1-C10 alkyl, and Ar⁵. In a further aspect, R⁵ is selected from halogen, —CF₃, C1-C8 alkyl, and Ar⁵. In a still further aspect, R⁵ is selected from halogen, C1-C4 alkyl, and Ar⁵. In yet a further aspect, R⁵ is selected from —F, —Cl, —Br, —CF₃, methyl, ethyl, n-propyl, isopropyl, and Ar⁵. In an even further aspect, R⁵ is selected from —F, —Cl, —Br, —CF₃, methyl, ethyl, and Ar⁵. In a still further aspect, R⁵ is selected from —F, —Cl, —Br, —CF₃, methyl, and Ar⁵.

In various aspects, R⁵ is selected from halogen and —CF₃. In yet a further aspect, R⁵ is selected from —F, —Cl, —Br, and —CF₃. In an even further aspect, R⁵ is selected from —F, —Cl, and —CF₃. In a still further aspect, R⁵ is selected from —F and —CF₃.

In a further aspect, R⁵ is —CF₃.

In various aspects, R⁵ is halogen. In yet a further aspect, R⁵ is selected from —F, —Cl, and —Br. In an even further aspect, R⁵ is selected from —F and —Cl. In a still further aspect, R⁵ is —F. In yet a further aspect, R⁵ is —Cl.

In various aspects, R⁵ is selected from halogen and C1-C10 alkyl. In a further aspect, R⁵ is selected from halogen and C1-C8 alkyl. In a still further aspect, R⁵ is selected from halogen and C1-C4 alkyl. In yet a further aspect, R⁵ is selected from —F, —Cl, —Br, methyl, ethyl, n-propyl, and isopropyl. In an even further aspect, R⁵ is selected from —F, —Cl, —Br, methyl, and ethyl. In a still further aspect, R⁵ is selected from —F, —Cl, —Br, and methyl.

In various aspects, R⁵ is C1-C10 alkyl. In a further aspect, R⁵ is C1-C8 alkyl. In a still further aspect, R⁵ is C1-C4 alkyl. In yet a further aspect, R⁵ is selected from methyl, ethyl, n-propyl, and isopropyl. In an even further aspect, R⁵ is selected from methyl and ethyl. In a still further aspect, R⁵ is methyl.

In various aspects, R⁵ is selected from halogen and Ar⁵. In a further aspect, R⁵ is selected from —F, —Cl, —Br, and Ar⁵. In an even further aspect, R⁵ is selected from —F, —Cl, and Ar⁵. In a still further aspect, R⁵ is selected from —F and Ar⁵.

In a further aspect, R⁵ is Ar⁵.

e. R¹⁰ Groups

In one aspect, R¹⁰, when present, is selected from C1-C30 alkyl, C2-C30 alkenyl, and —CH(NH₂)R²⁰. In a further aspect, R¹⁰, when present, is selected from C1-C15 alkyl, C2-C15 alkenyl, and —CH(NH₂)R²⁰. In a still further aspect, R¹⁰, when present, is selected from C1-C10 alkyl, C2-C10 alkenyl, and —CH(NH₂)R²⁰. In yet a further aspect, R¹⁰, when present, is selected from C1-C8 alkyl, C2-C8 alkenyl, and —CH(NH₂)R²⁰. In an even further aspect, R¹⁰, when present, is selected from C1-C4 alkyl, C2-C4 alkenyl, and —CH(NH₂)R²⁰. In a still further aspect, R¹⁰, when present, is selected from methyl, ethyl, n-propyl, isopropyl, ethenyl, propenyl, isopropenyl, and —CH(NH₂)R²⁰. In yet a further aspect, R¹⁰, when present, is selected from methyl, ethyl, ethenyl, and —CH(NH₂)R²⁰. In an even further aspect, R¹⁰, when present, is selected from methyl and —CH(NH₂)R²⁰.

In a further aspect, R¹⁰, when present, is —CH(NH₂)R²⁰.

In various aspects, R¹⁰, when present, is selected from C1-C30 alkyl and C2-C30 alkenyl. In a further aspect, R¹⁰, when present, is selected from C1-C15 alkyl and C2-C15 alkenyl. In a still further aspect, R¹⁰, when present, is selected from C1-C10 alkyl and C2-C10 alkenyl. In yet a further aspect, R¹⁰, when present, is selected from C1-C8 alkyl and C2-C8 alkenyl. In an even further aspect, R¹⁰, when present, is selected from C1-C4 alkyl and C2-C4 alkenyl. In a still further aspect, R¹⁰, when present, is selected from methyl, ethyl, n-propyl, isopropyl, ethenyl, propenyl, and isopropenyl. In yet a further aspect, R¹⁰, when present, is selected from methyl, ethyl, and ethenyl. In an even further aspect, R¹⁰, when present, is methyl.

In various aspects, R¹⁰, when present, is C2-C30 alkenyl. In a further aspect, R¹⁰, when present, is C2-C15 alkenyl. In a still further aspect, R¹⁰, when present, is C2-C10 alkenyl. In yet a further aspect, R¹⁰, when present, is C2-C8 alkenyl. In an even further aspect, R¹⁰, when present, is C2-C4 alkenyl. In a still further aspect, R¹⁰, when present, is selected from ethenyl, propenyl, and isopropenyl. In yet a further aspect, R¹⁰, when present, is ethenyl.

In various aspects, R¹⁰, when present, is C1-C30 alkyl. In a further aspect, R¹⁰, when present, is C1-C15 alkyl. In a still further aspect, R¹⁰, when present, is C1-C10 alkyl. In yet a further aspect, R¹⁰, when present, is C1-C8 alkyl. In an even further aspect, R¹⁰, when present, is C1-C4 alkyl. In a still further aspect, R¹⁰, when present, is selected from methyl, ethyl, n-propyl, and isopropyl. In yet a further aspect, R¹⁰, when present, is selected from methyl and ethyl.

f. R¹¹ and R^{11′} Groups

In one aspect, each of R¹¹ and R^{11′}, when present, is independently selected from hydrogen, C1-C4 alkyl, —(C1-C10 alkyl)CO₂(C1-C10 alkyl), —(C1-C10 alkoxy)CO₂(C1-C10 alkyl), —(C1-C10 alkyl)CO₂(C1-C10 alkylthiol), —(C1-C10 alkyl)-S—S—(C1-C10 alkyl), Ar¹, and —CH₂Ar¹. In a further aspect, each of R¹¹ and R^{11′}, when present, is independently selected from hydrogen, C1-C4 alkyl, —(C1-C8 alkyl)CO₂(C1-C8 alkyl), —(C1-C8 alkoxy)CO₂(C1-C8 alkyl), —(C1-C8 alkyl)CO₂(C1-C8 alkylthiol), —(C1-C8 alkyl)-S—S—(C1-C8 alkyl), Ar¹, and —CH₂Ar¹. In a still further aspect, each of R¹¹ and R^{11′}, when present, is independently selected from hydrogen, C1-C4 alkyl, —(C1-C4 alkyl)CO₂(C1-C4 alkyl), —(C1-C4 alkoxy)CO₂(C1-C4 alkyl), —(C1-C4 alkyl)CO₂(C1-C4 alkylthiol), —(C1-C4 alkyl)-S—S—(C1-C4 alkyl), Ar¹, and —CH₂Ar¹. In yet a further aspect, each of R¹¹ and R^{11′}, when present, is independently selected from hydrogen, methyl, ethyl, n-propyl, isopropyl, —CH₂CO₂CH₃, —CH₂CH₂CO₂CH₃, —CH₂CO₂CH₂CH₃, —CH₂CO₂CH₂CH₂CH₃, —CH₂CO₂CH(CH₃)₂, —OCH₂CO₂CH₃, —OCH₂CH₂CO₂CH₃, —OCH₂CO₂CH₂CH₃, —OCH₂CO₂CH₂CH₂CH₃, —OCH₂CO₂CH(CH₃)₂, —CH₂CO₂CH₂SH, —CH₂CH₂CO₂CH₂SH, —CH₂CO₂CH₂CH₂SH, —CH₂CO₂CH₂CH₂CH₂SH, —CH₂CO₂CH(CH₃)CH₂SH, —CH₂—S—S—CH₃, —CH₂CH₂—S—S—CH₃, —CH₂—S—S—CH₂CH₃, —CH₂—S—S—CH₂CH₂CH₃, —CH₂—S—S—CH(CH₃)₂, Ar, and —CH₂Ar¹. In an even further aspect, each of R¹¹ and R^{11′}, when present, is independently selected from hydrogen, methyl, ethyl, —CH₂CO₂CH₃, —CH₂CH₂CO₂CH₃, —CH₂CO₂CH₂CH₃, —OCH₂CO₂CH₃, —OCH₂CH₂CO₂CH₃, —OCH₂CO₂CH₂CH₃, —CH₂CO₂CH₂SH, —CH₂CH₂CO₂CH₂SH, —CH₂CO₂CH₂CH₂SH, —CH₂—S—S—CH₃, —CH₂CH₂—S—S—CH₃, —CH₂—S—S—CH₂CH₃, Ar, and —CH₂Ar¹. In a still further aspect, each of R¹¹ and R^{11′}, when present, is independently selected from hydrogen, methyl, —CH₂CO₂CH₃, —OCH₂CO₂CH₃, —CH₂CO₂CH₂SH, —CH₂—S—S—CH₃, Ar¹, and —CH₂Ar¹.

In a further aspect, each of R¹¹ and R^{11′}, when present, is hydrogen.

In various aspects, each of R¹¹ and R^{11′}, when present, is independently selected from hydrogen, C1-C4 alkyl, Ar¹, and —CH₂Ar¹. In a further aspect, each of R¹¹ and R^{11′}, when present, is independently selected from hydrogen, methyl, ethyl, n-propyl, isopropyl, Ar¹, and —CH₂Ar¹. In an even further aspect, each of R¹¹ and R^{11′}, when present, is independently selected from hydrogen, methyl, ethyl, Ar¹, and —CH₂Ar¹. In a still further aspect, each of R¹¹ and R^{11′}, when present, is independently selected from hydrogen, methyl, Ar¹, and —CH₂Ar¹.

In various aspects, each of R¹¹ and R^{11′}, when present, is independently selected from hydrogen and C1-C4 alky. In a further aspect, each of R¹¹ and R^{11′}, when present, is independently selected from hydrogen, methyl, ethyl, n-propyl, and isopropyl. In an even further aspect, each of R¹¹ and R^{11′}, when present, is independently selected from hydrogen, methyl, and ethyl. In a still further aspect, each of R¹¹ and R^{11′}, when present, is independently selected from hydrogen and methyl.

In various aspects, each of R¹¹ and R^{11′}, when present, is independently C1-C4 alky. In a further aspect, each of R¹¹ and R^{11′}, when present, is independently selected from methyl, ethyl, n-propyl, and isopropyl. In an even further aspect, each of R¹¹ and R^{11′}, when present, is independently selected from methyl and ethyl. In a still further aspect, each of R¹¹ and R^{11′}, when present, is methyl.

In various aspects, each of R¹¹ and R^{11′}, when present, is independently selected from hydrogen, Ar¹, and —CH₂Ar¹. In a further aspect, each of R¹¹ and R^{11′}, when present, is independently selected from hydrogen and —CH₂Ar¹. In an even further aspect, each of R¹¹ and R^{11′}, when present, is independently selected from hydrogen and Ar¹.

In various aspects, each of R¹¹ and R^{11′}, when present, is independently selected from Ar¹ and —CH₂Ar¹. In a further aspect, each of R¹¹ and R^{11′}, when present, is —CH₂Ar¹. In an even further aspect, each of R¹¹ and R^{11′}, when present, is Ar¹.

In various aspects, each of R¹¹ and R^{11′}, when present, is independently selected from hydrogen, —(C1-C10 alkyl)CO₂(C1-C10 alkyl), —(C1-C10 alkoxy)CO₂(C1-C10 alkyl), —(C1-C10 alkyl)CO₂(C1-C10 alkylthiol), and —(C1-C10 alkyl)-S—S—(C1-C10 alkyl). In a further aspect, each of R¹¹ and R^{11′}, when present, is independently selected from hydrogen, —(C1-C8 alkyl)CO₂(C1-C8 alkyl), —(C1-C8 alkoxy)CO₂(C1-C8 alkyl), —(C1-C8 alkyl)CO₂(C1-C8 alkylthiol), and —(C1-C8 alkyl)-S—S—(C1-C8 alkyl). In a still further aspect, each of R¹¹ and R^{11′}, when present, is independently selected from hydrogen, —(C1-C4 alkyl)CO₂(C1-C4 alkyl), —(C1-C4 alkoxy)CO₂(C1-C4 alkyl), —(C1-C4 alkyl)CO₂(C1-C4 alkylthiol), and —(C1-C4 alkyl)-S—S—(C1-C4 alkyl). In yet a further aspect, each of R¹¹ and R^{11′}, when present, is independently selected from hydrogen, —CH₂CO₂CH₃, —CH₂CH₂CO₂CH₃, —CH₂CO₂CH₂CH₃, —CH₂CO₂CH₂CH₂CH₃, —CH₂CO₂CH(CH₃)₂, —OCH₂CO₂CH₃, —OCH₂CH₂CO₂CH₃, —OCH₂CO₂CH₂CH₃, —OCH₂CO₂CH₂CH₂CH₃, —OCH₂CO₂CH(CH₃)₂, —CH₂CO₂CH₂SH, —CH₂CH₂CO₂CH₂SH, —CH₂CO₂CH₂CH₂SH, —CH₂CO₂CH₂CH₂CH₂SH, —CH₂CO₂CH(CH₃)CH₂SH, —CH₂—S—S—CH₃, —CH₂CH₂—S—S—CH₃, —CH₂—S—S—CH₂CH₃, —CH₂—S—S—CH₂CH₂CH₃, and —CH₂—S—S—CH(CH₃)₂. In an even further aspect, each of R¹¹ and R^{11′}, when present, is independently selected from hydrogen, —CH₂CO₂CH₃, —CH₂CH₂CO₂CH₃, —CH₂CO₂CH₂CH₃, —OCH₂CO₂CH₃, —OCH₂CH₂CO₂CH₃, —OCH₂CO₂CH₂CH₃, —CH₂CO₂CH₂SH, —CH₂CH₂CO₂CH₂SH, —CH₂CO₂CH₂CH₂SH, —CH₂—S—S—CH₃, —CH₂CH₂—S—S—CH₃, and —CH₂—S—S—CH₂CH₃. In a still further aspect, each of R¹¹ and R^{11′}, when present, is independently selected from hydrogen, —CH₂CO₂CH₃, —OCH₂CO₂CH₃, —CH₂CO₂CH₂SH, and —CH₂—S—S—CH₃.

g. R¹² and R^{12′} Groups

In one aspect, each of R¹² and R^{12′}, when present, is selected from —OR²¹ and —NHR²¹. In a further aspect, each of R¹² and R^{12′}, when present, is —OR²¹. In a still further aspect, each of R¹² and R^{12′}, when present, is —NHR²¹.

In a further aspect, one of R¹² and R^{12′}, when present, is —OR²¹, and the other of R¹² and R^{12′}, when present, is —NHR²¹.

h. R²⁰ Groups

In one aspect, R²⁰, when present, is selected from hydrogen, methyl, isopropyl, isobutyl, sec-butyl, —(CH₂)₃NHC(NH)NH₂, —(CH₂)₄NH₂, —CH₂CO₂H, —(CH₂)₂CO₂H, —CH₂OH, —CH(OH)CH₃, —CH₂C(O)NH₂, —(CH₂)₂C(O)NH₂, —CH₂SH, —(CH₂)₂SCH₃, —CH₂SeH, —CH₂C₆H₅, and —CH₂Cy¹. In a further aspect, R²⁰, when present, is selected from hydrogen, methyl, isopropyl, —(CH₂)₃NHC(NH)NH₂, —(CH₂)₄NH₂, —CH₂CO₂H, —(CH₂)₂CO₂H, —CH₂OH, —CH(OH)CH₃, —CH₂C(O)NH₂, —(CH₂)₂C(O)NH₂, —CH₂SH, —(CH₂)₂SCH₃, —CH₂SeH, —CH₂C₆H₅, and —CH₂Cy¹. In a still further aspect, R²⁰, when present, is selected from hydrogen, methyl, —(CH₂)₃NHC(NH)NH₂, —(CH₂)₄NH₂, —CH₂CO₂H, —(CH₂)₂CO₂H, —CH₂OH, —CH(OH)CH₃, —CH₂C(O)NH₂, —(CH₂)₂C(O)NH₂, —CH₂SH, —(CH₂)₂SCH₃, —CH₂SeH, —CH₂C₆H₅, and —CH₂Cy¹.

In a further aspect, R²⁰, when present, is hydrogen.

In a further aspect, R²⁰, when present, is selected from hydrogen, —CH₂SH, —(CH₂)₂SCH₃, and —CH₂SeH. In a still further aspect, R²⁰, when present, is selected from hydrogen, —CH₂SH, and —(CH₂)₂SCH. In yet a further aspect, R²⁰, when present, is selected from hydrogen and —CH₂SH. In an even further aspect, R²⁰, when present, is selected from hydrogen and —(CH₂)₂SCH. In a still further aspect, R²⁰, when present, is —CH₂SH. In yet a further aspect, R²⁰, when present, is —(CH₂)₂SCH.

In a further aspect, R²⁰, when present, is selected from hydrogen and —CH₂SeH. In a still further aspect, R²⁰, when present, is —CH₂SeH.

In a further aspect, R²⁰, when present, is selected from hydrogen, —CH₂CO₂H, —(CH₂)₂CO₂H, —CH₂OH, and —CH(OH)CH₃. In a still further aspect, R²⁰, when present, is selected from hydrogen, —CH₂CO₂H, and —(CH₂)₂CO₂H. In yet a further aspect, R²⁰, when present, is selected from hydrogen and —CH₂CO₂H. In an even further aspect, R²⁰, when present, is selected from hydrogen and —(CH₂)₂CO₂H. In a still further aspect,²⁰, when present, is —CH₂CO₂H. In yet a further aspect, R²⁰, when present, is —(CH₂)₂CO₂H.

In a further aspect, R²⁰, when present, is selected from hydrogen, —CH₂OH, and —CH(OH)CH₃. In a still further aspect, R²⁰, when present, is selected from hydrogen and —CH₂OH. In yet a further aspect, R²⁰, when present, is selected from hydrogen and —CH(OH)CH₃. In an even further aspect, R²⁰, when present, is —CH₂OH. In a still further aspect, R²⁰, when present, is —CH(OH)CH₃.

In a further aspect, R²⁰, when present, is selected from hydrogen, —(CH₂)₃NHC(NH)NH₂, —(CH₂)₄NH₂, —CH₂C(O)NH₂, and —(CH₂)₂C(O)NH₂. In a still further aspect, R²⁰, when present, is selected from hydrogen, —(CH₂)₃NHC(NH)NH₂, and —(CH₂)₄NH₂. In yet a further aspect, R²⁰, when present, is selected from hydrogen and —(CH₂)₄NH₂. In an even further aspect, R²⁰, when present, is selected from hydrogen and —(CH₂)₃NHC(NH)NH₂. In a still further aspect, R²⁰, when present, is —(CH₂)₄NH₂. In yet a further aspect, R²⁰, when present, is —(CH₂)₃NHC(NH)NH₂.

In a further aspect, R²⁰, when present, is selected from hydrogen, —CH₂C(O)NH₂, and —(CH₂)₂C(O)NH₂. In a still further aspect, R²⁰, when present, is selected from hydrogen and —(CH₂)₂C(O)NH₂. In yet a further aspect, R²⁰, when present, is selected from hydrogen and —CH₂C(O)NH₂. In an even further aspect, R²⁰, when present, is —(CH₂)₂C(O)NH₂. In a still further aspect, R²⁰, when present, is —CH₂C(O)NH₂.

In a further aspect, R²⁰, when present, is selected from hydrogen, —CH₂C₆H₅, and —CH₂Cy¹. In a still further aspect, R²⁰, when present, is selected from hydrogen and —CH₂Cy¹. In yet a further aspect, R²⁰, when present, is selected from hydrogen and —CH₂C₆H₅. In an even further aspect, R²⁰, when present, is —CH₂Cy¹. In a still further aspect, R²⁰, when present, is —CH₂C₆H₅.

In a further aspect, R²⁰, when present, is selected from hydrogen, methyl, isopropyl, isobutyl, and sec-butyl. In a still further aspect, R²⁰, when present, is selected from hydrogen, methyl, and isopropyl. In yet a further aspect, R²⁰, when present, is selected from hydrogen and methyl.

In various aspects, R²⁰, when present, is selected from methyl, isopropyl, isobutyl, and sec-butyl. In a still further aspect, R²⁰, when present, is selected from methyl and isopropyl. In yet a further aspect, R²⁰, when present, is methyl.

i. R²¹ Groups

In one aspect, each occurrence of R²¹, when present, is independently selected from hydrogen, —(C1-C10 alkyl)CO₂(C1-C10 alkyl), —(C1-C10 alkoxy)CO₂(C1-C10 alkyl), —(C1-C10 alkyl)CO₂(C1-C10 alkylthiol), —(C1-C10 alkyl)-S—S—(C1-C10 alkyl), Ar², —CH₂Ar², —P(O)OHOP(O)(OH)₂, and a structure represented by a formula:

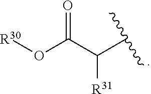

In a further aspect, each occurrence of R²¹, when present, is independently selected from hydrogen, —(C1-C8 alkyl)CO₂(C1-C8 alkyl), —(C1-C8 alkoxy)CO₂(C1-C8 alkyl), —(C1-C8 alkyl)CO₂(C1-C8 alkylthiol), —(C1-C8 alkyl)-S—S—(C1-C8 alkyl), Ar², —CH₂Ar², —P(O)OHOP(O)(OH)₂, and a structure represented by a formula:

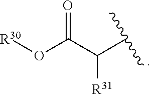

In a still further aspect, each occurrence of R²¹, when present, is independently selected from hydrogen, —(C1-C4 alkyl)CO₂(C1-C4 alkyl), —(C1-C4 alkoxy)CO₂(C1-C4 alkyl), —(C1-C4 alkyl)CO₂(C1-C4 alkylthiol), —(C1-C4 alkyl)-S—S—(C1-C4 alkyl), Ar², —CH₂Ar², —P(O)OHOP(O)(OH)₂, and a structure represented by a formula:

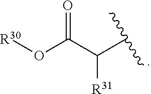

In yet a further aspect, each occurrence of R²¹, when present, is independently selected from hydrogen, —CH₂CO₂CH₃, —CH₂CH₂CO₂CH₃, —CH₂CO₂CH₂CH₃, —CH₂CO₂CH₂CH₂CH₃, —CH₂CO₂CH(CH₃)₂, —OCH₂CO₂CH₃, —OCH₂CH₂CO₂CH₃, —OCH₂CO₂CH₂CH₃, —OCH₂CO₂CH₂CH₂CH₃, —OCH₂CO₂CH(CH₃)₂, —CH₂CO₂CH₂SH, —CH₂CH₂CO₂CH₂SH, —CH₂CO₂CH₂CH₂SH, —CH₂CO₂CH₂CH₂CH₂SH, —CH₂CO₂CH(CH₃)CH₂SH, —CH₂—S—S—CH₃, —CH₂CH₂—S—S—CH₃, —CH₂—S—S—CH₂CH₃, —CH₂—S—S—CH₂CH₂CH₃, —CH₂—S—S—CH(CH₃)₂, Ar², —CH₂Ar², —P(O)OHOP(O)(OH)₂, and a structure represented by a formula:

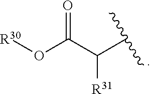

In an even further aspect, each occurrence of R²¹, when present, is independently selected from hydrogen, —CH₂CO₂CH₃, —CH₂CH₂CO₂CH₃, —CH₂CO₂CH₂CH₃, —OCH₂CO₂CH₃, —OCH₂CH₂CO₂CH₃, —OCH₂CO₂CH₂CH₃, —CH₂CO₂CH₂SH, —CH₂CH₂CO₂CH₂SH, —CH₂CO₂CH₂CH₂SH, —CH₂—S—S—CH₃, —CH₂CH₂—S—S—CH₃, —CH₂—S—S—CH₂CH₃, Ar², —CH₂Ar², —P(O)OHOP(O)(OH)₂, and a structure represented by a formula:

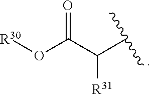

In a still further aspect, each occurrence of R²¹, when present, is independently selected from hydrogen, —CH₂CO₂CH₃, —OCH₂CO₂CH₃, —CH₂CO₂CH₂SH, —CH₂—S—S—CH₃, Ar², —CH₂Ar², —P(O)OHOP(O)(OH)₂, and a structure represented by a formula:

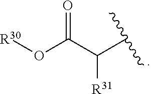

In a further aspect, each occurrence of R²¹, when present, is hydrogen.

In a further aspect, each occurrence of R²¹, when present, is independently selected from hydrogen and a structure represented by a formula:

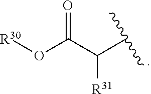

In a still further aspect, each occurrence of R²¹, when present, is a structure represented by a formula:

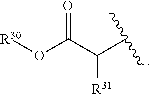

In a further aspect, each occurrence of R²¹, when present, is independently selected from hydrogen and —P(O)OHOP(O)(OH)₂. In a still further aspect, each occurrence of R²¹, when present, is —P(O)OHOP(O)(OH)₂.

In various aspects, each occurrence of R²¹, when present, is independently selected from Ar² and —CH₂Ar². In a further aspect, each occurrence of R²¹, when present, is —CH₂Ar². In an even further aspect, each occurrence of R²¹, when present, is Ar².

In various aspects, each occurrence of R²¹, when present, is independently selected from hydrogen, —(C1-C10 alkyl)CO₂(C1-C10 alkyl), —(C1-C10 alkoxy)CO₂(C1-C10 alkyl), —(C1-C10 alkyl)CO₂(C1-C10 alkylthiol), and —(C1-C10 alkyl)-S—S—(C1-C10 alkyl). In a further aspect, each occurrence of R²¹, when present, is independently selected from hydrogen, —(C1-C8 alkyl)CO₂(C1-C8 alkyl), —(C1-C8 alkoxy)CO₂(C1-C8 alkyl), —(C1-C8 alkyl)CO₂(C1-C8 alkylthiol), and —(C1-C8 alkyl)-S—S—(C1-C8 alkyl). In a still further aspect, each occurrence of R²¹, when present, is independently selected from hydrogen, —(C1-C4 alkyl)CO₂(C1-C4 alkyl), —(C1-C4 alkoxy)CO₂(C1-C4 alkyl), —(C1-C4 alkyl)CO₂(C1-C4 alkylthiol), and —(C1-C4 alkyl)-S—S—(C1-C4 alkyl). In yet a further aspect, each occurrence of R²¹, when present, is independently selected from hydrogen, —CH₂CO₂CH₃, —CH₂CH₂CO₂CH₃, —CH₂CO₂CH₂CH₃, —CH₂CO₂CH₂CH₂CH₃, —CH₂CO₂CH(CH₃)₂, —OCH₂CO₂CH₃, —OCH₂CH₂CO₂CH₃, —OCH₂CO₂CH₂CH₃, —OCH₂CO₂CH₂CH₂CH₃, —OCH₂CO₂CH(CH₃)₂, —CH₂CO₂CH₂SH, —CH₂CH₂CO₂CH₂SH, —CH₂CO₂CH₂CH₂SH, —CH₂CO₂CH₂CH₂CH₂SH, —CH₂CO₂CH(CH₃)CH₂SH, —CH₂—S—S—CH₃, —CH₂CH₂—S—S—CH₃, —CH₂—S—S—CH₂CH₃, —CH₂—S—S—CH₂CH₂CH₃, and —CH₂—S—S—CH(CH₃)₂. In an even further aspect, each occurrence of R²¹, when present, is independently selected from hydrogen, —CH₂CO₂CH₃, —CH₂CH₂CO₂CH₃, —CH₂CO₂CH₂CH₃, —OCH₂CO₂CH₃, —OCH₂CH₂CO₂CH₃, —OCH₂CO₂CH₂CH₃, —CH₂CO₂CH₂SH, —CH₂CH₂CO₂CH₂SH, —CH₂CO₂CH₂CH₂SH, —CH₂—S—S—CH₃, —CH₂CH₂—S—S—CH₃, and —CH₂—S—S—CH₂CH₃. In a still further aspect, each occurrence of R²¹, when present, is independently selected from hydrogen, —CH₂CO₂CH₃, —OCH₂CO₂CH₃, —CH₂CO₂CH₂SH, and —CH₂—S—S—CH₃.

j. R³⁰ Groups

In one aspect, each occurrence of R³⁰, when present, is independently selected from hydrogen, C1-C8 alkyl, Cy², and —CH₂Cy². In a further aspect, each occurrence of R³⁰, when present, is independently selected from hydrogen, C1-C4 alkyl, Cy², and -CH₂Cy². In a still further aspect, each occurrence of R³⁰, when present, is independently selected from hydrogen, methyl, ethyl, n-propyl, isopropyl, Cy², and —CH₂Cy². In yet a further aspect, each occurrence of R³⁰, when present, is independently selected from hydrogen, methyl, ethyl, Cy², and —CH₂Cy². In an even further aspect, each occurrence of R³⁰, when present, is independently selected from hydrogen, methyl, Cy², and —CH₂Cy².

In various aspects, each occurrence of R³⁰, when present, is independently selected from hydrogen, Cy², and —CH₂Cy². In a further aspect, each occurrence of R³⁰, when present, is independently selected from hydrogen and Cy². In a still further aspect, each occurrence of R³⁰, when present, is independently selected from hydrogen and —CH₂Cy².

In various aspects, each occurrence of R³⁰, when present, is independently selected from Cy² and —CH₂Cy². In a further aspect, each occurrence of R³⁰, when present, is Cy². In a still further aspect, each occurrence of R³⁰, when present, is —CH₂Cy².

In various aspects, each occurrence of R³⁰, when present, is independently selected from hydrogen and C1-C8 alkyl. In a further aspect, each occurrence of R³⁰, when present, is independently selected from hydrogen and C1-C4 alkyl. In a still further aspect, each occurrence of R³⁰, when present, is independently selected from hydrogen, methyl, ethyl, n-propyl, and isopropyl. In yet a further aspect, each occurrence of R³⁰, when present, is independently selected from hydrogen, methyl, and ethyl. In an even further aspect, each occurrence of R³⁰, when present, is independently selected from hydrogen and ethyl. In a still further aspect, each occurrence of R³⁰, when present, is independently selected from hydrogen and methyl.

In a further aspect, each occurrence of R³⁰, when present, is hydrogen.

In various aspects, each occurrence of R³⁰, when present, is independently C1-C8 alkyl. In a further aspect, each occurrence of R³⁰, when present, is independently C1-C4 alkyl. In a still further aspect, each occurrence of R³⁰, when present, is independently selected from methyl, ethyl, n-propyl, and isopropyl. In yet a further aspect, each occurrence of R³⁰, when present, is independently selected from methyl and ethyl. In an even further aspect, each occurrence of R³⁰, when present, is ethyl. In a still further aspect, each occurrence of R³⁰, when present, is methyl.

k. R³¹ Groups

In one aspect, each occurrence of R³¹, when present, is independently selected from hydrogen and C1-C8 alkyl. In a further aspect, each occurrence of R³¹, when present, is independently selected from hydrogen and C1-C4 alkyl. In a still further aspect, each occurrence of R³¹, when present, is independently selected from hydrogen, methyl, ethyl, n-propyl, and isopropyl. In yet a further aspect, each occurrence of R³¹, when present, is independently selected from hydrogen, methyl, and ethyl. In an even further aspect, each occurrence of R³¹, when present, is independently selected from hydrogen and ethyl. In a still further aspect, each occurrence of R³¹, when present, is independently selected from hydrogen and methyl.

In a further aspect, each occurrence of R³¹, when present, is hydrogen.

In various aspects, each occurrence of R³¹, when present, is independently C1-C8 alkyl. In a further aspect, each occurrence of R³¹, when present, is independently C1-C4 alkyl. In a still further aspect, each occurrence of R³¹, when present, is independently selected from methyl, ethyl, n-propyl, and isopropyl. In yet a further aspect, each occurrence of R³¹, when present, is independently selected from methyl and ethyl. In an even further aspect, each occurrence of R³¹, when present, is ethyl. In a still further aspect, each occurrence of R³¹, when present, is methyl.

l. R^{32a} and R^{32b} Groups

In one aspect, each of R^{32a} and R^{32b}, when present, is independently selected from hydrogen and C1-C4 alkyl. In a further aspect, each of R^{32a} and R^{32b}, when present, is independently selected from hydrogen, methyl, ethyl, n-propyl, and isopropyl. In a still further aspect, each of R^{32a} and R^{32b}, when present, is independently selected from hydrogen, methyl, and ethyl. In yet a further aspect, each of R^{32a} and R^{32b}, when present, is independently selected from hydrogen and ethyl. In an even further aspect, each of R^{32a} and R^{32b}, when present, is independently selected from hydrogen and methyl.

In a further aspect, each of R^{32a} and R^{32b}, when present, is hydrogen.

In various aspects, each of R^{32a} and R^{32b}, when present, is independently C1-C4 alkyl. In a further aspect, each of R^{32a} and R^{32b}, when present, is independently selected from methyl, ethyl, n-propyl, and isopropyl. In a still further aspect, each of R^{32a} and R^{32b}, when present, is independently selected from methyl and ethyl. In yet a further aspect, each of R^{32a} and R^{32b}, when present, is ethyl. In an even further aspect, each of R^{32a} and R^{32b}, when present, is methyl.

m. AR¹ Groups

In one aspect, each occurrence of Ar¹, when present, is independently selected from aryl and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, each occurrence of Ar¹, when present, is independently selected from aryl and heteroaryl, and is substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, each occurrence of Ar¹, when present, is independently selected from aryl and heteroaryl, and is substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, each occurrence of Ar¹, when present, is independently selected from aryl and heteroaryl, and is monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, each occurrence of Ar¹, when present, is independently selected from aryl and heteroaryl, and is unsubstituted.

In various aspects, each occurrence of Ar¹, when present, is independently aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. Examples of aryls include, but are not limited to, phenyl, naphthyl, phenanthrenyl, anthracenyl, and pyrenyl. In a further aspect, each occurrence of Ar¹, when present, is independently aryl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, each occurrence of Ar¹, when present, is independently aryl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, each occurrence of Ar¹, when present, is independently aryl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, each occurrence of Ar¹, when present, is independently unsubstituted aryl.

In various aspects, each occurrence of Ar¹, when present, is independently phenyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, each occurrence of Ar¹, when present, is independently phenyl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, each occurrence of Ar¹, when present, is independently phenyl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, each occurrence of Ar¹, when present, is independently phenyl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, each occurrence of Ar¹, when present, is unsubstituted phenyl.

In various aspects, each occurrence of Ar¹, when present, is independently heteroaryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. Examples of heteroaryls include, but are not limited to, pyrrole, furan, thiophene, pyridine, pyridazine, pyrimidine, pyrazine, triazine, indole, indazole, benzimidazole, azaindazole, purine, benzofuran, benzo[b]thiophene, benzo[d]oxazole, and benzo[d]isothiazole. In a further aspect, each occurrence of Ar¹, when present, is independently heteroaryl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, each occurrence of Ar¹, when present, is independently heteroaryl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, each occurrence of Ar¹, when present, is independently heteroaryl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, each occurrence of Ar¹, when present, is independently unsubstituted heteroaryl.

In various aspects, each occurrence of Ar¹, when present, is independently pyridinyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, each occurrence of Ar¹, when present, is independently pyridinyl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, each occurrence of Ar¹, when present, is independently pyridinyl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, each occurrence of Ar¹, when present, is independently pyridinyl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, each occurrence of Ar¹, when present, is independently unsubstituted pyridinyl.

n. Ar² Groups

In one aspect, each occurrence of Ar², when present, is independently selected from aryl and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, each occurrence of Ar², when present, is independently selected from aryl and heteroaryl, and is substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, each occurrence of Ar², when present, is independently selected from aryl and heteroaryl, and is substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, each occurrence of Ar², when present, is independently selected from aryl and heteroaryl, and is monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, each occurrence of Ar², when present, is independently selected from aryl and heteroaryl, and is unsubstituted.

In various aspects, each occurrence of Ar², when present, is independently aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. Examples of aryls include, but are not limited to, phenyl, naphthyl, phenanthrenyl, anthracenyl, and pyrenyl. In a further aspect, each occurrence of Ar², when present, is independently aryl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, each occurrence of Ar², when present, is independently aryl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, each occurrence of Ar², when present, is independently aryl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, each occurrence of Ar², when present, is independently unsubstituted aryl.

In various aspects, each occurrence of Ar², when present, is independently phenyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, each occurrence of Ar², when present, is independently phenyl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, each occurrence of Ar², when present, is independently phenyl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, each occurrence of Ar², when present, is independently phenyl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, each occurrence of Ar², when present, is unsubstituted phenyl.

In various aspects, each occurrence of Ar², when present, is independently heteroaryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. Examples of heteroaryls include, but are not limited to, pyrrole, furan, thiophene, pyridine, pyridazine, pyrimidine, pyrazine, triazine, indole, indazole, benzimidazole, azaindazole, purine, benzofuran, benzo[b]thiophene, benzo[d]oxazole, and benzo[d]isothiazole. In a further aspect, each occurrence of Ar², when present, is independently heteroaryl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, each occurrence of Ar², when present, is independently heteroaryl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, each occurrence of Ar², when present, is independently heteroaryl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, each occurrence of Ar², when present, is independently unsubstituted heteroaryl.

In various aspects, each occurrence of Ar², when present, is independently pyridinyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, each occurrence of Ar², when present, is independently pyridinyl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, each occurrence of Ar², when present, is independently pyridinyl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, each occurrence of Ar², when present, is independently pyridinyl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, each occurrence of Ar², when present, is independently unsubstituted pyridinyl.

o. Ar³ Groups

In one aspect, Ar³, when present, is selected from aryl and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, Ar³, when present, is selected from aryl and heteroaryl, and is substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar³, when present, is selected from aryl and heteroaryl, and is substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Ar³, when present, is selected from aryl and heteroaryl, and is monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Ar³, when present, is selected from aryl and heteroaryl, and is unsubstituted.

In various aspects, Ar³, when present, is aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. Examples of aryls include, but are not limited to, phenyl, naphthyl, phenanthrenyl, anthracenyl, and pyrenyl. In a further aspect, Ar³, when present, is aryl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar³, when present, is aryl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Ar³, when present, is aryl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Ar³, when present, is unsubstituted aryl.

In various aspects, Ar³, when present, is phenyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, Ar³, when present, is phenyl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar³, when present, is phenyl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Ar³, when present, is phenyl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Ar³, when present, is unsubstituted phenyl.

In various aspects, Ar³, when present, is heteroaryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. Examples of heteroaryls include, but are not limited to, pyrrole, furan, thiophene, pyridine, pyridazine, pyrimidine, pyrazine, triazine, indole, indazole, benzimidazole, azaindazole, purine, benzofuran, benzo[b]thiophene, benzo[d]oxazole, and benzo[d]isothiazole. In a further aspect, Ar³, when present, is heteroaryl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar³, when present, is heteroaryl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Ar³, when present, is heteroaryl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Ar³, when present, is unsubstituted heteroaryl.

In various aspects, Ar³, when present, is pyridinyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, Ar³, when present, is pyridinyl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar³, when present, is pyridinyl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Ar³, when present, is pyridinyl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Ar³, when present, is unsubstituted pyridinyl.

p. Ar⁴ Groups

In one aspect, Ar⁴, when present, is selected from aryl and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, Ar⁴, when present, is selected from aryl and heteroaryl, and is substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar⁴, when present, is selected from aryl and heteroaryl, and is substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Ar⁴, when present, is selected from aryl and heteroaryl, and is monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Ar⁴, when present, is selected from aryl and heteroaryl, and is unsubstituted.

In various aspects, Ar⁴, when present, is aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. Examples of aryls include, but are not limited to, phenyl, naphthyl, phenanthrenyl, anthracenyl, and pyrenyl. In a further aspect, Ar⁴, when present, is aryl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar⁴, when present, is aryl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Ar⁴, when present, is aryl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Ar⁴, when present, is unsubstituted aryl.

In various aspects, Ar⁴, when present, is phenyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, Ar⁴, when present, is phenyl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar⁴, when present, is phenyl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Ar⁴, when present, is phenyl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Ar⁴, when present, is unsubstituted phenyl.

In various aspects, Ar⁴, when present, is heteroaryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. Examples of heteroaryls include, but are not limited to, pyrrole, furan, thiophene, pyridine, pyridazine, pyrimidine, pyrazine, triazine, indole, indazole, benzimidazole, azaindazole, purine, benzofuran, benzo[b]thiophene, benzo[d]oxazole, and benzo[d]isothiazole. In a further aspect, Ar⁴, when present, is heteroaryl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar⁴, when present, is heteroaryl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Ar⁴, when present, is heteroaryl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Ar⁴, when present, is unsubstituted heteroaryl.

In various aspects, Ar⁴, when present, is pyridinyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, Ar⁴, when present, is pyridinyl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar⁴, when present, is pyridinyl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Ar⁴, when present, is pyridinyl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Ar⁴, when present, is unsubstituted pyridinyl.

q. Ar⁵ Groups

In one aspect, Ar⁵, when present, is selected from aryl and heteroaryl, and is substituted with 0, 1, 2, or 3 groups halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, Ar⁵, when present, is selected from aryl and heteroaryl, and is substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar⁵, when present, is selected from aryl and heteroaryl, and is substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Ar⁵, when present, is selected from aryl and heteroaryl, and is monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Ar⁵, when present, is selected from aryl and heteroaryl, and is unsubstituted.

In various aspects, Ar⁵, when present, is aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. Examples of aryls include, but are not limited to, phenyl, naphthyl, phenanthrenyl, anthracenyl, and pyrenyl. In a further aspect, Ar⁵, when present, is aryl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar⁵, when present, is aryl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Ar⁵, when present, is aryl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Ar⁵, when present, is unsubstituted aryl.

In various aspects, Ar⁵, when present, is phenyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, Ar⁵, when present, is phenyl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar⁵, when present, is phenyl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Ar⁵, when present, is phenyl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Ar⁵, when present, is unsubstituted phenyl.

In various aspects, Ar⁵, when present, is heteroaryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. Examples of heteroaryls include, but are not limited to, pyrrole, furan, thiophene, pyridine, pyridazine, pyrimidine, pyrazine, triazine, indole, indazole, benzimidazole, azaindazole, purine, benzofuran, benzo[b]thiophene, benzo[d]oxazole, and benzo[d]isothiazole. In a further aspect, Ar⁵, when present, is heteroaryl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar⁵, when present, is heteroaryl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Ar⁵, when present, is heteroaryl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Ar⁵, when present, is unsubstituted heteroaryl.

In various aspects, Ar⁵, when present, is pyridinyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, Ar⁵, when present, is pyridinyl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar⁵, when present, is pyridinyl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Ar⁵, when present, is pyridinyl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Ar⁵, when present, is unsubstituted pyridinyl.

r. Cy¹ Groups

In one aspect, Cy¹, when present, is selected from monocyclic aryl, para-hydroxy monocyclic aryl, 4-imidazolyl, and 3-indolyl. In a further aspect, Cy¹, when present, is selected from phenyl, para-hydroxy phenyl, 4-imidazolyl, and 3-indolyl.

In a further aspect, Cy¹, when present, is selected from monocyclic aryl and para-hydroxy monocyclic aryl. In a still further aspect, Cy¹, when present, is monocyclic aryl. In yet a further aspect, Cy¹, when present, is para-hydroxy monocyclic aryl.

In a further aspect, Cy¹, when present, is selected from phenyl and para-hydroxy phenyl. In a still further aspect, Cy¹, when present, is phenyl. In yet a further aspect, Cy¹, when present, is para-hydroxy phenyl.

In a further aspect, Cy¹, when present, is selected from 4-imidazolyl and 3-indolyl. In a still further aspect, Cy¹, when present, is 4-imidazolyl. In yet a further aspect, Cy¹, when present, is 3-indolyl.

In various aspects, Cy¹, when present, is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, Cy¹, when present, is substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Cy¹, when present, is substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Cy¹, when present, is monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Cy¹, when present, is unsubstituted.

s. Cy² Groups

In one aspect, each occurrence of Cy², when present, is independently selected from C3-C6 cycloalkyl, aryl, and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, each occurrence of Cy², when present, is independently selected from C3-C6 cycloalkyl, aryl, and heteroaryl, and is substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, each occurrence of Cy², when present, is independently selected from C3-C6 cycloalkyl, aryl, and heteroaryl, and is substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, each occurrence of Cy², when present, is independently selected from C3-C6 cycloalkyl, aryl, and heteroaryl, and is monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, each occurrence of Cy², when present, is independently selected from C3-C6 cycloalkyl, aryl, and heteroaryl, and is unsubstituted.

In various aspects, each occurrence of Cy², when present, is independently C3-C6 cycloalkyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, each occurrence of Cy², when present, is independently C3-C6 cycloalkyl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, each occurrence of Cy², when present, is independently C3-C6 cycloalkyl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, each occurrence of Cy², when present, is independently C3-C6 cycloalkyl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, each occurrence of Cy², when present, is independently unsubstituted C3-C6 cycloalkyl.

In various aspects, each occurrence of Cy², when present, is independently aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. Examples of aryls include, but are not limited to, phenyl, naphthyl, phenanthrenyl, anthracenyl, and pyrenyl. In a further aspect, each occurrence of Cy², when present, is independently aryl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, each occurrence of Cy², when present, is independently aryl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, each occurrence of Cy², when present, is independently aryl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, each occurrence of Cy², when present, is independently unsubstituted aryl.

In various aspects, each occurrence of Cy², when present, is independently phenyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, each occurrence of Cy², when present, is independently phenyl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, each occurrence of Cy², when present, is independently phenyl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, each occurrence of Cy², when present, is independently phenyl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, each occurrence of Cy², when present, is independently unsubstituted phenyl.

In various aspects, each occurrence of Cy², when present, is independently heteroaryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. Examples of heteroaryls include, but are not limited to, pyrrole, furan, thiophene, pyridine, pyridazine, pyrimidine, pyrazine, triazine, indole, indazole, benzimidazole, azaindazole, purine, benzofuran, benzo[b]thiophene, benzo[d]oxazole, and benzo[d]isothiazole. In a further aspect, each occurrence of Cy², when present, is independently heteroaryl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, each occurrence of Cy², when present, is independently heteroaryl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, each occurrence of Cy², when present, is independently heteroaryl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, each occurrence of Cy², when present, is independently unsubstituted heteroaryl.

In various aspects, each occurrence of Cy², when present, is independently tetrahydrofuranyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, each occurrence of Cy², when present, is independently tetrahydrofuranyl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, each occurrence of Cy², when present, is independently tetrahydrofuranyl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, each occurrence of Cy², when present, is independently tetrahydrofuranyl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, each occurrence of Cy², when present, is independently unsubstituted tetrahydrofuranyl.

t. Cy³ Groups

In one aspect, Cy³, when present, is C3-C6 cycloalkyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, Cy³, when present, is C3-C6 cycloalkyl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Cy³, when present, is C3-C6 cycloalkyl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Cy³, when present, is C3-C6 cycloalkyl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Cy³, when present, is unsubstituted C3-C6 cycloalkyl.

In one aspect, Cy³, when present, is cyclopropyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, Cy³, when present, is cyclopropyl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Cy³, when present, is cyclopropyl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Cy³, when present, is cyclopropyl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Cy³, when present, is unsubstituted cyclopropyl.

In one aspect, Cy³, when present, is cyclobutyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, Cy³, when present, is cyclobutyl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Cy³, when present, is cyclobutyl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Cy³, when present, is cyclobutyl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Cy³, when present, is unsubstituted cyclobutyl.

In one aspect, Cy³, when present, is cyclopentyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, Cy³, when present, is cyclopentyl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Cy³, when present, is cyclopentyl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Cy³, when present, is cyclopentyl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Cy³, when present, is unsubstituted cyclopentyl.

In one aspect, Cy³, when present, is cyclohexyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, Cy³, when present, is cyclohexyl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Cy³, when present, is cyclohexyl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Cy³, when present, is cyclohexyl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Cy³, when present, is unsubstituted cyclohexyl.

### 2. Example Compounds

In one aspect, a compound can be present as one or more of the following structures:

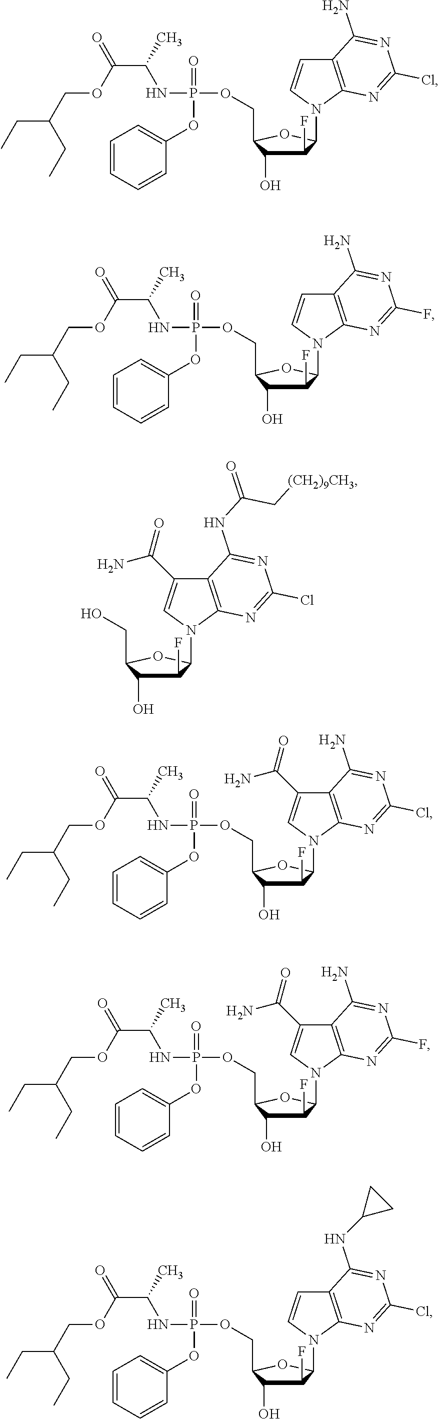

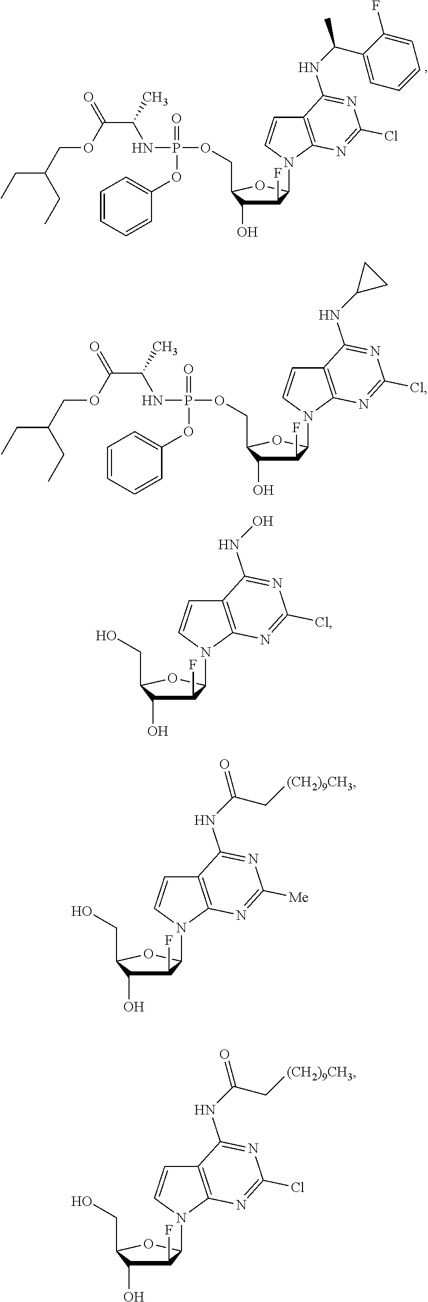

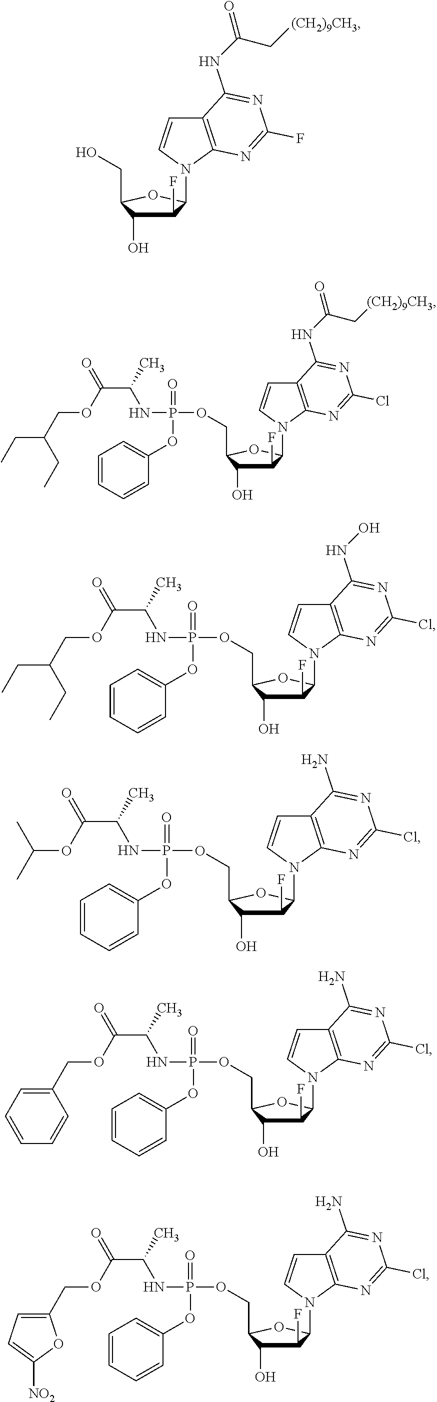

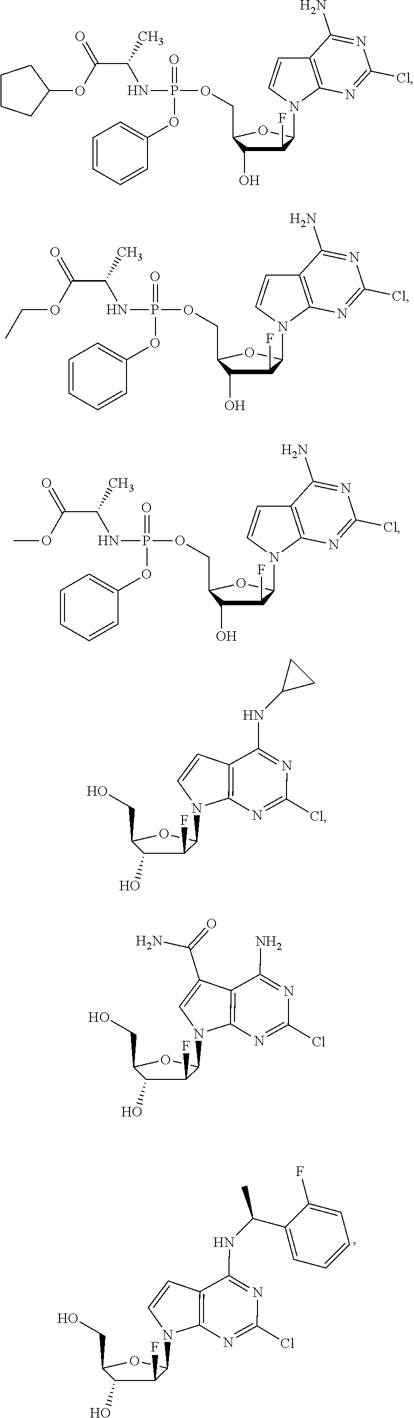

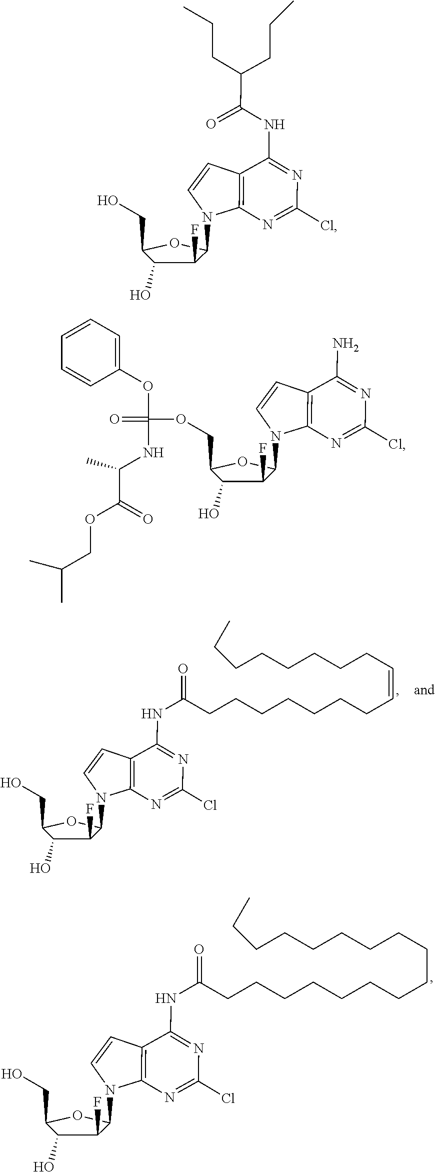

or a pharmaceutically acceptable salt thereof.

### C. Pharmaceutical Compositions

In one aspect, disclosed are pharmaceutical compositions comprising a disclosed compound, or a pharmaceutically acceptable salt thereof and a pharmaceutically acceptable carrier.

In one aspect, disclosed are pharmaceutical compositions comprising a pharmaceutically acceptable carrier and a therapeutically effective amount of at least one compound having a structure represented by a formula:

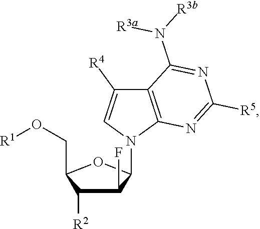

wherein R¹ is selected from hydrogen, —C(O)R¹⁰, —P(O)(OR¹¹)₂, and —P(O)(OR¹¹)R¹²; wherein R² is selected from hydrogen, —OH, C1-C8 alkoxy, —P(O)(OR^{11′})₂, and —P(O)(OR^{11′})R^{12′}; wherein R¹⁰, when present, is selected from C1-C30 alkyl, C2-C30 alkenyl, and —CH(NH₂)R²⁰; wherein R²⁰, when present, is selected from hydrogen, methyl, isopropyl, isobutyl, sec-butyl, —(CH₂)₃NHC(NH)NH₂, —(CH₂)₄NH₂, —CH₂CO₂H, —(CH₂)₂CO₂H, —CH₂OH, —CH(OH)CH₃, —CH₂C(O)NH₂, —(CH₂)₂C(O)NH₂, —CH₂SH, —(CH₂)₂SCH₃, —CH₂SeH, —CH₂C₆H₅, and —CH₂Cy¹; wherein Cy¹, when present, is selected from monocyclic aryl, para-hydroxy monocyclic aryl, 4-imidazolyl, and 3-indolyl; wherein each of R¹¹ and R^{11′}, when present, is independently selected from hydrogen, C1-C4 alkyl, —(C1-C10 alkyl)CO₂(C1-C10 alkyl), —(C1-C10 alkoxy)CO₂(C1-C10 alkyl), —(C1-C10 alkyl)CO₂(C1-C10 alkylthiol), —(C1-C10 alkyl)-S—S—(C1-C10 alkyl), Ar¹, and —CH₂Ar¹; wherein each occurrence of Ar¹, when present, is selected from aryl and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R¹² and R^{12′}, when present, is selected from —OR²¹ and —NHR²¹; wherein each occurrence of R²¹, when present, is selected from hydrogen, —(C1-C10 alkyl)CO₂(C1-C10 alkyl), —(C1-C10 alkoxy)CO₂(C1-C10 alkyl), —(C1-C10 alkyl)CO₂(C1-C10 alkylthiol), —(C1-C10 alkyl)-S—S—(C1-C10 alkyl), Ar², —CH₂Ar², —P(O)OHOP(O)(OH)₂, and a structure represented by a formula:

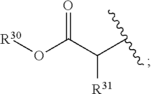

wherein each occurrence of R³⁰, when present, is independently selected from hydrogen, C1-C8 alkyl, Cy², and —CH₂Cy²; wherein each occurrence of Cy², when present, is independently selected from C3-C6 cycloalkyl, aryl, and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each occurrence of R³¹, when present, is independently selected from hydrogen and C1-C8 alkyl; and wherein each occurrence of Ar², when present, is independently selected from aryl and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; or wherein each of R¹ and R² together comprise a structure represented by a formula:

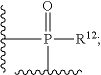

wherein each of R^{3a} and R^{3b} is independently selected from hydrogen, —OH, C1-C10 alkoxy, C1-C8 alkyl, —C(O)(C1-C30 alkyl), —C(O)(C2-C30 alkenyl), Cy³, —CR^{32a}R^{32b}Ar³; wherein each of R^{32a} and R^{32b}, when present, is independently selected from hydrogen and C1-C4 alkyl; wherein Cy³, when present, is C3-C6 cycloalkyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein Ar³, when present, is selected from aryl and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R⁴ is selected from hydrogen, halogen, —CN, —C(O)NH₂, —CO₂H, —COMe, —SO₂Me, C1-C4 haloalkyl, and Ar⁴; wherein Ar⁴, when present, is selected from aryl and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R⁵ is selected from halogen, —CF₃, C1-C10 alkyl, and Ar⁵; and wherein Ar⁵, when present, is selected from aryl and heteroaryl, and is substituted with 0, 1, 2, or 3 groups halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, or a pharmaceutically acceptable salt thereof.

In various aspects, the compounds and compositions of the invention can be administered in pharmaceutical compositions, which are formulated according to the intended method of administration. The compounds and compositions described herein can be formulated in a conventional manner using one or more physiologically acceptable carriers or excipients. For example, a pharmaceutical composition can be formulated for local or systemic administration, e.g., administration by drops or injection into the ear, insufflation (such as into the ear), intravenous, topical, or oral administration.

The nature of the pharmaceutical compositions for administration is dependent on the mode of administration and can readily be determined by one of ordinary skill in the art. In various aspects, the pharmaceutical composition is sterile or sterilizable. The therapeutic compositions featured in the invention can contain carriers or excipients, many of which are known to skilled artisans. Excipients that can be used include buffers (for example, citrate buffer, phosphate buffer, acetate buffer, and bicarbonate buffer), amino acids, urea, alcohols, ascorbic acid, phospholipids, polypeptides (for example, serum albumin), EDTA, sodium chloride, liposomes, mannitol, sorbitol, water, and glycerol. The nucleic acids, polypeptides, small molecules, and other modulatory compounds featured in the invention can be administered by any standard route of administration. For example, administration can be parenteral, intravenous, subcutaneous, or oral. A modulatory compound can be formulated in various ways, according to the corresponding route of administration. For example, liquid solutions can be made for administration by drops into the ear, for injection, or for ingestion; gels or powders can be made for ingestion or topical application. Methods for making such formulations are well known and can be found in, for example, Remington's Pharmaceutical Sciences, 18th Ed., Gennaro, ed., Mack Publishing Co., Easton, Pa. 1990.

In various aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In various aspects, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise a compound of the invention (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouthwashes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In a further aspect, an effective amount is a therapeutically effective amount. In a still further aspect, an effective amount is a prophylactically effective amount.

In a further aspect, the pharmaceutical composition is administered to a mammal. In a still further aspect, the mammal is a human. In an even further aspect, the human is a patient.

In a further aspect, the pharmaceutical composition is used to treat a viral infection such as, for example, human immunodeficiency virus (HIV), human papillomavirus (HPV), herpes simplex virus (HSV), human cytomegalovirus (HCMV), chicken pox, infectious mononucleosis, mumps, measles, rubella, shingles, ebola, viral gastroenteritis, viral hepatitis, viral meningitis, human metapneumovirus, human parainfluenza virus type 1, parainfluenza virus type 2, parainfluenza virus type 3, respiratory syncytial virus, viral pneumonia, Chikungunya virus (CHIKV), Venezuelan equine encephalitis (VEEV), dengue (DENV), influenza, West Nile virus (WNV), and zika (ZIKV). In a still further aspect, the viral infection is viral hepatitis. In yet a further aspect, the viral hepatitis is hepatitis B virus.

It is understood that the disclosed compositions can be prepared from the disclosed compounds. It is also understood that the disclosed compositions can be employed in the disclosed methods of using.

### D. Methods of Making a Compound

The compounds of this invention can be prepared by employing reactions as shown in the following schemes, in addition to other standard manipulations that are known in the literature, exemplified in the experimental sections or clear to one skilled in the art. For clarity, examples having a single substituent are shown where multiple substituents are allowed under the definitions disclosed herein.

Reactions used to generate the compounds of this invention are prepared by employing reactions as shown in the following Reaction Schemes, as described and exemplified below. In certain specific examples, the disclosed compounds can be prepared by Routes I-IV, as described and exemplified below. The following examples are provided so that the invention might be more fully understood, are illustrative only, and should not be construed as limiting.

1. Route I

In one aspect, 2,4,7-substituted-7-deaza-2′-deoxy-2′-fluoroarabinosyl nucleoside and nucleotide prodrugs can be prepared as shown below.

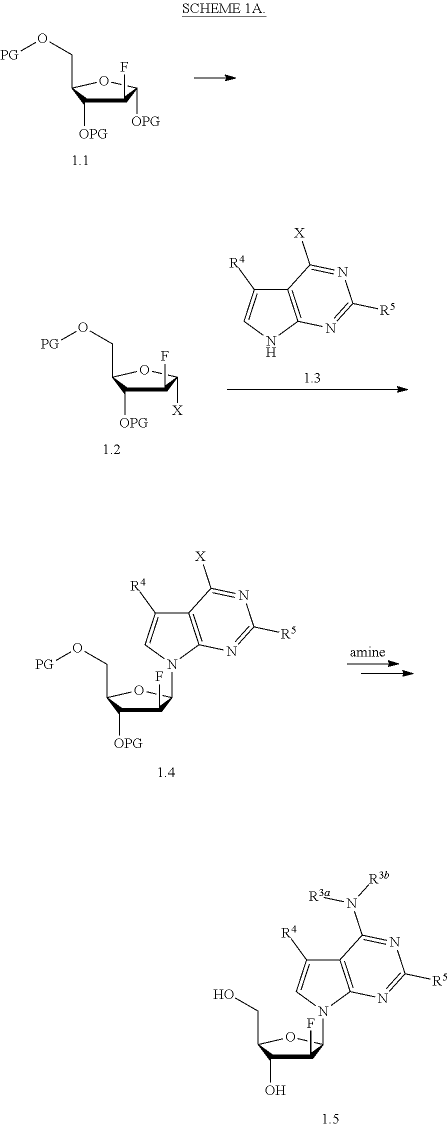

Compounds are represented in generic form, wherein each occurrence of PG is independently an alcohol protecting group, each occurrence of X is independently a halogen, and with substituents as noted in compound descriptions elsewhere herein. A more specific example is set forth below.

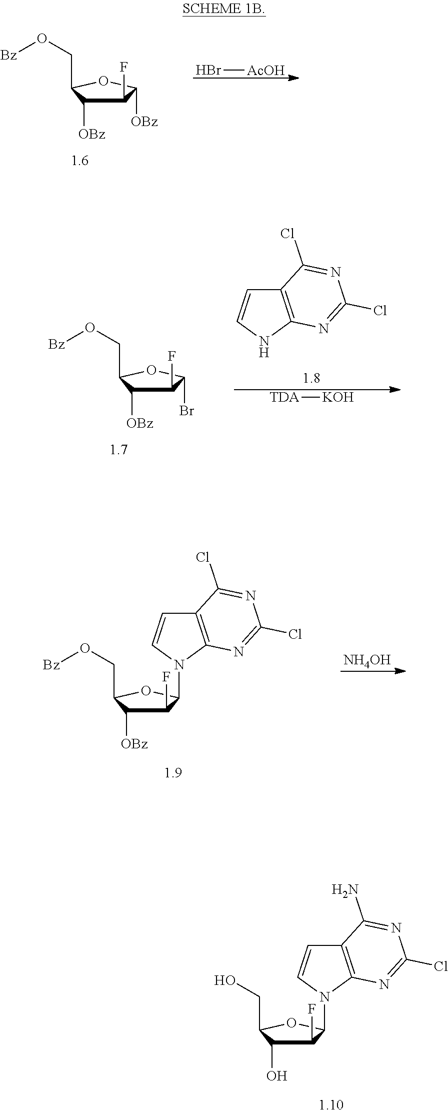

In one aspect, compounds of type 1.10, and similar compounds, can be prepared according to reaction Scheme 1B above. Thus, compounds of type 1.7 can be prepared by a substitution reaction between an appropriate sugar, e.g., 1.6 as shown above, and an appropriate halide, e.g., hydrogen bromide solution in acetic acid (HBr-AcOH) as shown above. Appropriate sugars are commercially available or prepared by methods known to one skilled in the art. Compounds of type 1.9 can be prepared by displacement of an appropriate halide, e.g., 1.7 as shown above, with an appropriate pyrimidine base, e.g., 1.8 as shown above. The displacement is carried out in the presence of an appropriate base, e.g., tris(3,6-dioxaheptyl)amine (TDA) and potassium hydroxide (KOH). Appropriate pyrimidine bases are commercially available or prepared by methods known to one skilled in the art. Compounds of type 1.10 can be prepared by a substitution/deprotection reaction (simultaneously as shown above or sequentially) of an appropriate nucleoside, e.g., 1.9 as shown above. The substitution/deprotection reaction is carried out in the presence of an appropriate amine and/or deprotecting agent, e.g., ammonium hydroxide as shown above. As can be appreciated by one skilled in the art, the above reaction provides an example of a generalized approach wherein compounds similar in structure to the specific reactants above (compounds similar to compounds of type 1.1, 1.2, 1.3, and 1.4), can be substituted in the reaction to provide 2,4,7-substituted-7-deaza-2′-deoxy-2′-fluoroarabinosyl nucleoside prodrug analogs similar to Formula 1.5.

2. Route II

In one aspect, 2,4,7-substituted-7-deaza-2′-deoxy-2′-fluoroarabinosyl nucleoside and nucleotide prodrugs can be prepared as shown below.

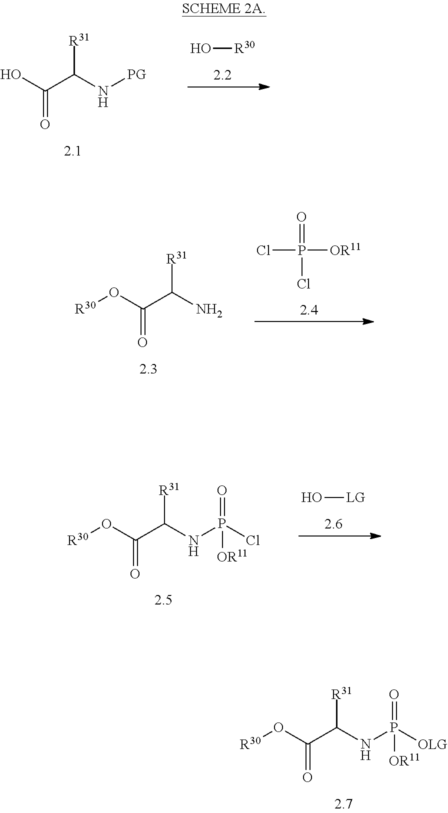

Compounds are represented in generic form, wherein PG is an amine protecting group, LG is a leaving group, and with substituents as noted in compound descriptions elsewhere herein. A more specific example is set forth below.

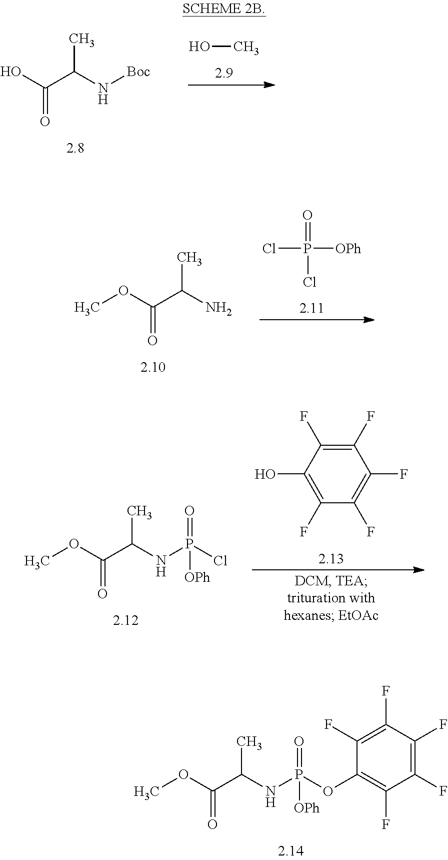

In one aspect, compounds of type 2.14 and similar compounds, can be prepared according to reaction Scheme 2B above. Thus, compounds of type 2.10 can be prepared by O-alkylation/deprotection (simultaneously as shown above or sequentially) between an appropriate protected amine, e.g., 2.8 as shown above, and an appropriate alcohol, e.g., 2.9 as shown above. Appropriate protected amines and appropriate alcohols are commercially available or prepared by methods known to one skilled in the art. The O-alkylation/deprotection is carried out in the presence of an appropriate solvent, e.g., dichloromethane (DCM) as shown above, and an appropriate deprotecting agent, e.g., trimethylsilyl chloride (TMSCl) as shown above. Compounds of type 2.12 can be prepared by phosphorylation of an appropriate amine, e.g., 2.10 as shown above, with an appropriate phosphinate, e.g., 2.11 as shown above. Appropriate phosphinates are commercially available or prepared by methods known to one skilled in the art. Compounds of type 2.14 can be prepared by displacement of an appropriate halide, e.g., 2.12 as shown above, with an appropriate aryl alcohol, e.g., 2.13 as shown above. Appropriate aryl alcohols are commercially available or prepared by methods known to one skilled in the art. The displacement is carried out in the presence of an appropriate base, e.g., triethylamine (TEA) as shown above, in an appropriate solvent, e.g., dichloromethane (DCM) as shown above. As can be appreciated by one skilled in the art, the above reaction provides an example of a generalized approach wherein compounds similar in structure to the specific reactants above (compounds similar to compounds of type 2.1, 2.2, 2.3, 2.4, and 2.5), can be substituted in the reaction to provide substrates similar to Formula 2.6 for the preparation of 2,4,7-substituted-7-deaza-2′-deoxy-2′-fluoroarabinosyl nucleoside/nucleotide prodrug analogs.

3. Route III

In one aspect, 2,4,7-substituted-7-deaza-2′-deoxy-2′-fluoroarabinosyl nucleoside and nucleotide prodrugs can be prepared as shown below.

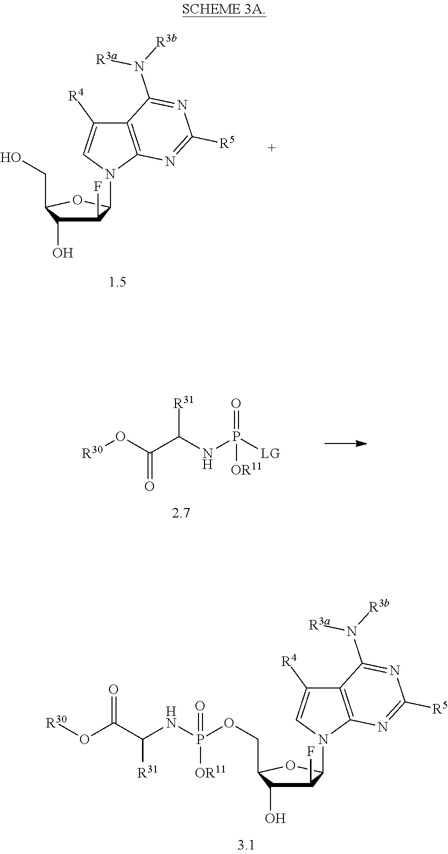

Compounds are represented in generic form, wherein LG is a leaving group and with substituents as noted in compound descriptions elsewhere herein. A more specific example is set forth below.

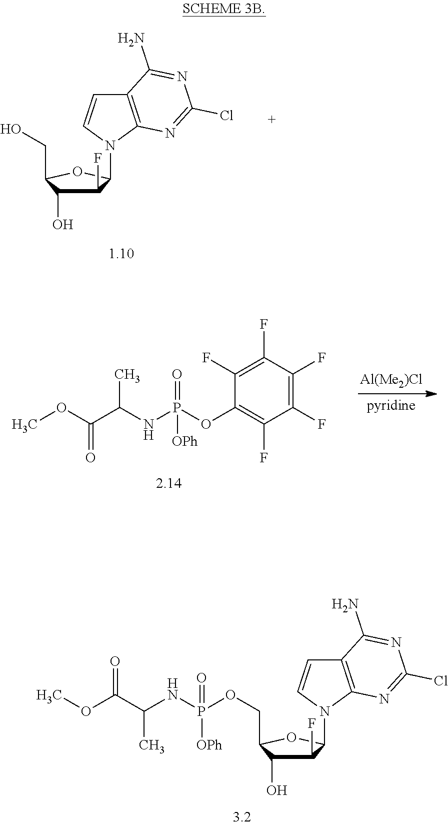

In one aspect, compounds of type 3.2, and similar compounds, can be prepared according to reaction Scheme 3B above. Thus, compounds of type 3.2 can be prepared by a substitution reaction between an appropriate nucleoside, e.g., 1.10 as shown above, and an appropriate phosphonate, e.g., 2.14 as shown above. The substitution reaction is carried out in the presence of an appropriate Lewis acid, e.g., dimethylaluminum chloride as shown above, and an appropriate base, e.g., pyridine as shown above. As can be appreciated by one skilled in the art, the above reaction provides an example of a generalized approach wherein compounds similar in structure to the specific reactants above (compounds similar to compounds of type 1.5 and 2.7), can be substituted in the reaction to provide 2,4,7-substituted-7-deaza-2′-deoxy-2′-fluoroarabinosyl nucleotide prodrug analogs similar to Formula 3.2.

4. Route IV

In one aspect, 2,4,7-substituted-7-deaza-2′-deoxy-2′-fluoroarabinosyl nucleoside and nucleotide prodrugs can be prepared as shown below.

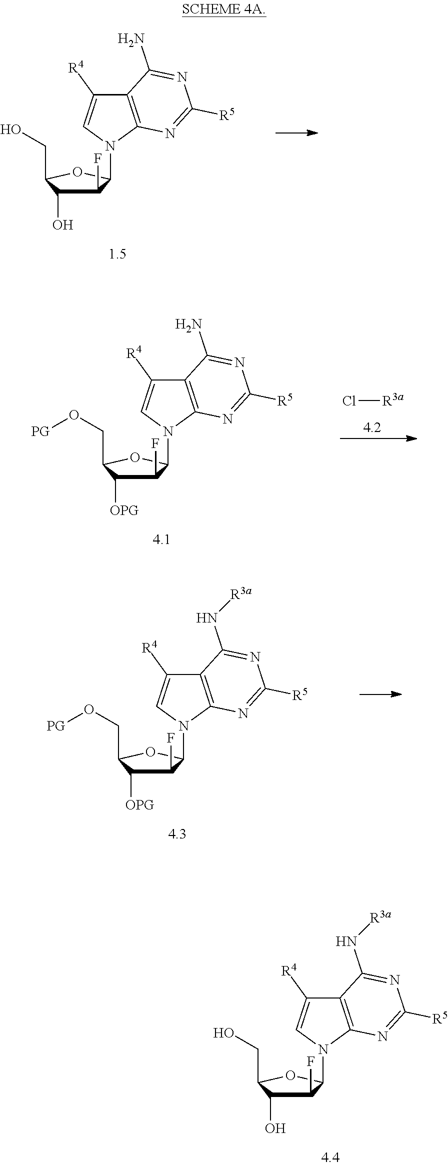

Compounds are represented in generic form, wherein each occurrence of PG is independently an alcohol protecting group and with substituents as noted in compound descriptions elsewhere herein. A more specific example is set forth below.

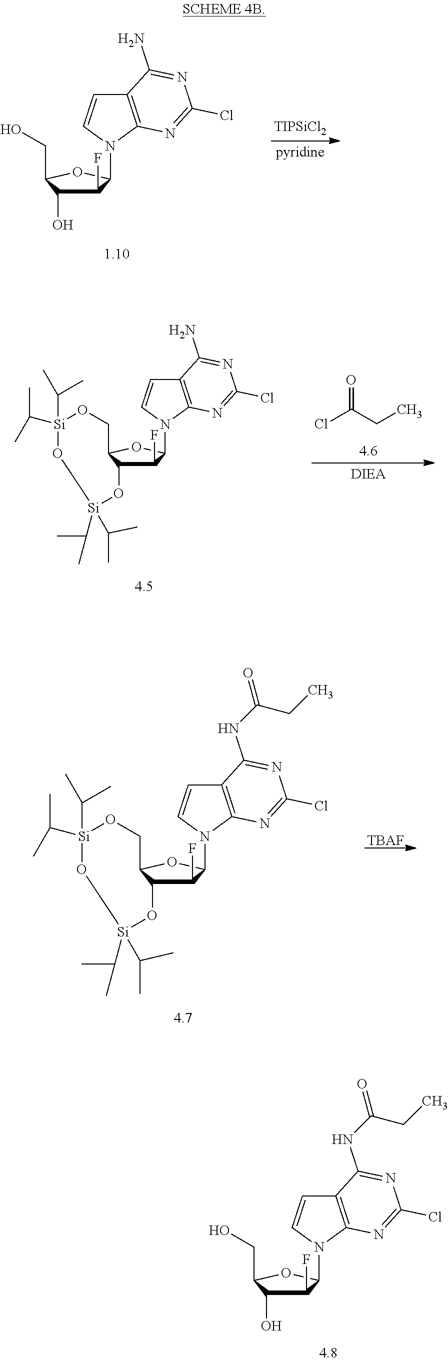

In one aspect, compounds of type 4.8, and similar compounds, can be prepared according to reaction Scheme 4B above. Thus, compounds of type 4.5 can be prepared by protection of an appropriate alcohol, e.g., 1.10 as shown above. The protection is carried out in the presence of an appropriate protecting agent, e.g., 1,3-dichloro-1,1,3,3-tetraisopropyldisiloxane, and an appropriate base, e.g., pyridine. Compounds of type 4.7 can be prepared by acylation of an appropriate amine, e.g., 4.5 as shown above. The acylation is carried out in the presence of an appropriate acyl halide, e.g., 4.6 as shown above, and an appropriate base, e.g., N,N-diisopropylethylamine (DIEA). Appropriate acyl halides are commercially available or prepared by methods known to one skilled in the art. As would be understood by one skilled in the art, similar protocols could be followed to alkylate amine 4.5 as desired. Compounds of type 4.8 can be prepared by deprotection of an appropriate nucleoside, e.g., 4.7 as shown above. The deprotection is carried out in the presence of an appropriate deprotecting agent, e.g., tetra-n-butylammonium fluoride (TBAF) as shown above. As can be appreciated by one skilled in the art, the above reaction provides an example of a generalized approach wherein compounds similar in structure to the specific reactants above (compounds similar to compounds of type 1.5, 4.1, 4.2, and 4.3), can be substituted in the reaction to provide 2,4,7-substituted-7-deaza-2′-deoxy-2′-fluoroarabinosyl nucleoside prodrug analogs similar to Formula 4.4.

### E. Methods of Using the Compounds

The compounds and pharmaceutical compositions of the invention are useful in treating or controlling disorders associated with a viral infection, in particular, viral hepatitis or herpes simplex virus.

Examples of viral infections for which the compounds and compositions can be useful in treating, include, but are not limited to, human immunodeficiency virus (HIV), human papillomavirus (HPV), herpes simplex virus (HSV), human cytomegalovirus (HCMV), chicken pox, infectious mononucleosis, mumps, measles, rubella, shingles, ebola, viral gastroenteritis, viral hepatitis, viral meningitis, human metapneumovirus, human parainfluenza virus type 1, parainfluenza virus type 2, parainfluenza virus type 3, respiratory syncytial virus, viral pneumonia, Chikungunya virus (CHIKV), Venezuelan equine encephalitis (VEEV), dengue (DENV), influenza, West Nile virus (WNV), and zika (ZIKV).

To treat or control the disorder, the compounds and pharmaceutical compositions comprising the compounds are administered to a subject in need thereof, such as a vertebrate, e.g., a mammal, a fish, a bird, a reptile, or an amphibian. The subject can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. The subject is preferably a mammal, such as a human. Prior to administering the compounds or compositions, the subject can be diagnosed with a need for treatment of a viral infection, such as, for example, viral hepatitis or herpes simplex virus.

The compounds or compositions can be administered to the subject according to any method. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration and parenteral administration, including injectable such as intravenous administration, intra-arterial administration, intramuscular administration, and subcutaneous administration. Administration can be continuous or intermittent. A preparation can be administered therapeutically; that is, administered to treat an existing disease or condition. A preparation can also be administered prophylactically; that is, administered for prevention of a viral infection, such as, for example, viral hepatitis or herpes simplex virus.

The therapeutically effective amount or dosage of the compound can vary within wide limits. Such a dosage is adjusted to the individual requirements in each particular case including the specific compound(s) being administered, the route of administration, the condition being treated, as well as the patient being treated. In general, in the case of oral or parenteral administration to adult humans weighing approximately 70 Kg or more, a daily dosage of about 10 mg to about 10,000 mg, preferably from about 200 mg to about 1,000 mg, should be appropriate, although the upper limit may be exceeded. The daily dosage can be administered as a single dose or in divided doses, or for parenteral administration, as a continuous infusion. Single dose compositions can contain such amounts or submultiples thereof of the compound or composition to make up the daily dose. The dosage can be adjusted by the individual physician in the event of any contraindications. Dosage can vary, and can be administered in one or more dose administrations daily, for one or several days.

1. Treatment Methods

The compounds disclosed herein are useful for treating or controlling disorders associated with a viral infection, in particular, human immunodeficiency virus (HIV), human papillomavirus (HPV), herpes simplex virus (HSV), human cytomegalovirus (HCMV), chicken pox, infectious mononucleosis, mumps, measles, rubella, shingles, ebola, viral gastroenteritis, viral hepatitis, viral meningitis, human metapneumovirus, human parainfluenza virus type 1, parainfluenza virus type 2, parainfluenza virus type 3, respiratory syncytial virus, viral pneumonia, Chikungunya virus (CHIKV), Venezuelan equine encephalitis (VEEV), dengue (DENV), influenza, West Nile virus (WNV), and zika (ZIKV). Thus, provided is a method comprising administering a therapeutically effective amount of a composition comprising a disclosed compound to a subject. In a further aspect, the method can be a method for treating a viral infection.

a. Treating a Viral Infection

In one aspect, disclosed are methods of treating a viral infection in a subject having the viral infection, the method comprising the step of administering to the subject a therapeutically effective amount of at least one disclosed compound, or a pharmaceutically acceptable salt thereof.

In one aspect, disclosed are methods for the treatment of a viral infection in a subject having the viral infection, the method comprising the step of administering to the subject a therapeutically effective amount of at least one compound having a structure represented by a formula:

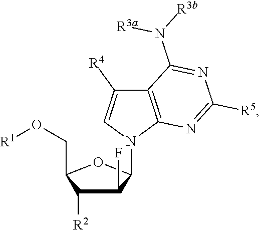

wherein R¹ is selected from hydrogen, —C(O)R¹⁰, —P(O)(OR¹¹)₂, and —P(O)(OR¹¹)R¹²; wherein R² is selected from hydrogen, —OH, C1-C8 alkoxy, —P(O)(OR^{11′})₂, and —P(O)(OR^{11′})R^{12′}; wherein R¹⁰, when present, is selected from C1-C30 alkyl, C2-C30 alkenyl, and —CH(NH₂)R²⁰; wherein R²⁰, when present, is selected from hydrogen, methyl, isopropyl, isobutyl, sec-butyl, —(CH₂)₃NHC(NH)NH₂, —(CH₂)₄NH₂, —CH₂CO₂H, —(CH₂)₂CO₂H, —CH₂OH, —CH(OH)CH₃, —CH₂C(O)NH₂, —(CH₂)₂C(O)NH₂, —CH₂SH, —(CH₂)₂SCH₃, —CH₂SeH, —CH₂C₆H₅, and -CH₂Cy¹; wherein Cy¹, when present, is selected from monocyclic aryl, para-hydroxy monocyclic aryl, 4-imidazolyl, and 3-indolyl; wherein each of R¹¹ and R^{11′}, when present, is independently selected from hydrogen, C1-C4 alkyl, —(C1-C10 alkyl)CO₂(C1-C10 alkyl), —(C1-C10 alkoxy)CO₂(C1-C10 alkyl), —(C1-C10 alkyl)CO₂(C1-C10 alkylthiol), —(C1-C10 alkyl)-S—S—(C1-C10 alkyl), Ar¹, and —CH₂Ar¹; wherein each occurrence of Ar¹, when present, is selected from aryl and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R¹² and R^{12′}, when present, is selected from —OR²¹ and —NHR²¹; wherein each occurrence of R²¹, when present, is selected from hydrogen, —(C1-C10 alkyl)CO₂(C1-C10 alkyl), —(C1-C10 alkoxy)CO₂(C1-C10 alkyl), —(C1-C10 alkyl)CO₂(C1-C10 alkylthiol), —(C1-C10 alkyl)-S—S—(C1-C10 alkyl), Ar², —CH₂Ar², —P(O)OHOP(O)(OH)₂, and a structure represented by a formula:

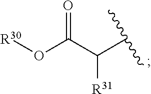

wherein each occurrence of R³⁰, when present, is independently selected from hydrogen, C1-C8 alkyl, Cy², and —CH₂Cy²; wherein each occurrence of Cy², when present, is independently selected from C3-C6 cycloalkyl, aryl, and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each occurrence of R³¹, when present, is independently selected from hydrogen and C1-C8 alkyl; and wherein each occurrence of Ar², when present, is independently selected from aryl and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; or wherein each of R¹ and R² together comprise a structure represented by a formula:

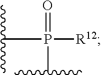

wherein each of R^{3a} and R^{3b} is independently selected from hydrogen, —OH, C1-C10 alkoxy, C1-C8 alkyl, —C(O)(C1-C30 alkyl), —C(O)(C2-C30 alkenyl), Cy³, —CR^{32a}R^{32b}Ar³; wherein each of R^{32a} and R^{32b}, when present, is independently selected from hydrogen and C1-C4 alkyl; wherein Cy³, when present, is C3-C6 cycloalkyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein Ar³, when present, is selected from aryl and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R⁴ is selected from hydrogen, halogen, —CN, —C(O)NH₂, —CO₂H, —COMe, —SO₂Me, C1-C4 haloalkyl, and Ar⁴; wherein Ar⁴, when present, is selected from aryl and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R⁵ is selected from halogen, —CF₃, C1-C10 alkyl, and Ar⁵; and wherein Ar⁵, when present, is selected from aryl and heteroaryl, and is substituted with 0, 1, 2, or 3 groups halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, or a pharmaceutically acceptable salt thereof.

Examples of viral infections include, but are not limited to, human immunodeficiency virus (HIV), human papillomavirus (HPV), herpes simplex virus (HSV), human cytomegalovirus (HCMV), chicken pox, infectious mononucleosis, mumps, measles, rubella, shingles, ebola, viral gastroenteritis, viral hepatitis, viral meningitis, human metapneumovirus, human parainfluenza virus type 1, parainfluenza virus type 2, parainfluenza virus type 3, respiratory syncytial virus, viral pneumonia, Chikungunya virus (CHIKV), Venezuelan equine encephalitis (VEEV), dengue (DENV), influenza, West Nile virus (WNV), and zika (ZIKV).

In a further aspect, the subject has been diagnosed with a need for treatment of the disorder prior to the administering step.

In a further aspect, the subject is a mammal. In a still further aspect, the mammal is a human.

In a further aspect, the method further comprises the step of identifying a subject in need of treatment of the viral infection.

In a further aspect, the effective amount is a therapeutically effective amount. In a still further aspect, the effective amount is a prophylactically effective amount.

In a further aspect, the disorder is associated with a viral infection. In a still further aspect, the viral infection is selected from human immunodeficiency virus (HIV), human papillomavirus (HPV), herpes simplex virus (HSV), human cytomegalovirus (HCMV), chicken pox, infectious mononucleosis, mumps, measles, rubella, shingles, ebola, viral gastroenteritis, viral hepatitis, viral meningitis, human metapneumovirus, human parainfluenza virus type 1, parainfluenza virus type 2, parainfluenza virus type 3, respiratory syncytial virus, viral pneumonia, Chikungunya virus (CHIKV), Venezuelan equine encephalitis (VEEV), dengue (DENV), influenza, West Nile virus (WNV), zika (ZIKV), 229E, NL63, OC43, HKU1, Middle East respiratory syndrome coronavirus (MERS-CoV), severe acute respiratory syndrome coronavirus (SARS-CoV), and severe acute respiratory syndrome coronavirus disease 2019 (SARS-CoV-2). In an even further aspect, the viral infection is viral hepatitis. In a still further aspect, the viral hepatitis is hepatitis B virus. In yet a further aspect, the viral infection is herpes simplex virus.

In a further aspect, the method further comprises the step of administering a therapeutically effective amount of at least one antiviral agent. In a still further aspect, the at least one agent is selected from acemannan, acyclovir, acyclovir sodium, adamantanamine, adefovir, adenine arabinoside, alovudine, alvircept sudotox, amantadine hydrochloride, aranotin, arildone, atevirdine mesylate, avridine, cidofovir, cipamfylline, cytarabine hydrochloride, BMS 806, C31G, carrageenan, cellulose sulfate, cyclodextrins, dapivirine, delavirdine mesylate, desciclovir, dextrin 2-sulfate, didanosine, disoxaril, dolutegravir, edoxudine, enviradene, envirozime, etravirine, famciclovir, famotine hydrochloride, fiacitabine, fialuridine, fosarilate, foscarnet sodium, fosfonet sodium, FTC, ganciclovir, ganciclovir sodium, GSK 1265744, 9-2-hydroxy-ethoxy methylguanine, ibalizumab, idoxuridine, interferon, 5-iodo-2′-deoxyuridine, IQP-0528, kethoxal, lamivudine, lobucavir, maraviroc, memotine pirodavir, penciclovir, raltegravir, ribavirin, rimantadine hydrochloride, rilpivirine (TMC-278), saquinavir mesylate, SCH-C, SCH-D, somantadine hydrochloride, sorivudine, statolon, stavudine, T20, tilorone hydrochloride, TMC120, TMC125, trifluridine, trifluorothymidine, tenofovir, tenofovir alefenamide, tenofovir disoproxyl fumarate, prodrugs of tenofovir, UC-781, UK-427, UK-857, valacyclovir, valacyclovir hydrochloride, vidarabine, vidarabine phosphate, vidarabine sodium phosphate, viroxime, zalcitabene, zidovudine, and zinviroxime.

In a further aspect, the at least one compound and the at least one agent are administered sequentially. In a still further aspect, the at least one compound and the at least one agent are administered simultaneously.

In a further aspect, the at least one compound and the at least one agent are co-formulated. In a still further aspect, the at least one compound and the at least one agent are co-packaged.

2. Methods of Inhibiting a Viral Infection in a Mammal

In one aspect, disclosed are methods of inhibiting a viral infection in a mammal, the method comprising the step of administering to the mammal a therapeutically effective amount of at least one disclosed compound, or a pharmaceutically acceptable salt thereof.

Thus, in one aspect, disclosed are methods of inhibiting a viral infection in a mammal, the method comprising the step of administering to the mammal a therapeutically effective amount of at least one compound having a structure represented by a formula:

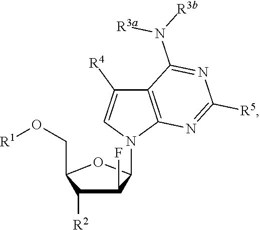

wherein R¹ is selected from hydrogen, —C(O)R¹⁰, —P(O)(OR¹¹)₂, and —P(O)(OR¹¹)R¹²; wherein R² is selected from hydrogen, —OH, C1-C8 alkoxy, —P(O)(OR¹¹)₂, and —P(O)(OR^{11′})R^{12′}; wherein R¹⁰, when present, is selected from C1-C30 alkyl, C2-C30 alkenyl, and —CH(NH₂)R²⁰; wherein R²⁰, when present, is selected from hydrogen, methyl, isopropyl, isobutyl, sec-butyl, —(CH₂)₃NHC(NH)NH₂, —(CH₂)₄NH₂, —CH₂CO₂H, —(CH₂)₂CO₂H, —CH₂OH, —CH(OH)CH₃, —CH₂C(O)NH₂, —(CH₂)₂C(O)NH₂, —CH₂SH, —(CH₂)₂SCH₃, —CH₂SeH, —CH₂C₆H₅, and —CH₂Cy¹; wherein Cy¹, when present, is selected from monocyclic aryl, para-hydroxy monocyclic aryl, 4-imidazolyl, and 3-indolyl; wherein each of R¹¹ and R^{11′}, when present, is independently selected from hydrogen, C1-C4 alkyl, —(C1-C10 alkyl)CO₂(C1-C10 alkyl), —(C1-C10 alkoxy)CO₂(C1-C10 alkyl), —(C1-C10 alkyl)CO₂(C1-C10 alkylthiol), —(C1-C10 alkyl)-S—S—(C1-C10 alkyl), Ar¹, and —CH₂Ar¹; wherein each occurrence of Ar¹, when present, is selected from aryl and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R¹² and R^{12′}, when present, is selected from —OR²¹ and —NHR²¹; wherein each occurrence of R²¹, when present, is selected from hydrogen, —(C1-C10 alkyl)CO₂(C1-C10 alkyl), —(C1-C10 alkoxy)CO₂(C1-C10 alkyl), —(C1-C10 alkyl)CO₂(C1-C10 alkylthiol), —(C1-C10 alkyl)-S—S—(C1-C10 alkyl), Ar², —CH₂Ar², —P(O)OHOP(O)(OH)₂, and a structure represented by a formula:

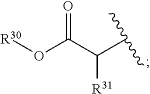

wherein each occurrence of R³⁰, when present, is independently selected from hydrogen, C1-C8 alkyl, Cy², and —CH₂Cy²; wherein each occurrence of Cy², when present, is independently selected from C3-C6 cycloalkyl, aryl, and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each occurrence of R³¹, when present, is independently selected from hydrogen and C1-C8 alkyl; and wherein each occurrence of Ar², when present, is independently selected from aryl and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; or wherein each of R¹ and R² together comprise a structure represented by a formula:

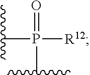

wherein each of R^{3a} and R^{3b} is independently selected from hydrogen, —OH, C1-C10 alkoxy, C1-C8 alkyl, —C(O)(C1-C30 alkyl), —C(O)(C2-C30 alkenyl), Cy³, —CR^{32a}R^{32b}Ar³; wherein each of R^{32a} and R^{32b}, when present, is independently selected from hydrogen and C1-C4 alkyl; wherein Cy³, when present, is C3-C6 cycloalkyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein Ar³, when present, is selected from aryl and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R⁴ is selected from hydrogen, halogen, —CN, —C(O)NH₂, —CO₂H, —COMe, —SO₂Me, C1-C4 haloalkyl, and Ar⁴; wherein Ar⁴, when present, is selected from aryl and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R⁵ is selected from halogen, —CF₃, C1-C10 alkyl, and Ar⁵; and wherein Ar⁵, when present, is selected from aryl and heteroaryl, and is substituted with 0, 1, 2, or 3 groups halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, or a pharmaceutically acceptable salt thereof.

In a further aspect, the compound exhibits inhibition of a viral infection. In a still further aspect, the compound exhibits a decrease in a viral infection. In yet a further aspect, the viral infection is viral hepatitis such as, for example, hepatitis B virus or herpes simplex virus.

In a further aspect, the compound exhibits inhibition of viral hepatitis activity with an IC₅₀ of less than about 30 μM. In a still further aspect, the compound exhibits inhibition of viral hepatitis activity with an IC₅₀ of less than about 25 μM. In yet a further aspect, the compound exhibits inhibition of viral hepatitis activity with an IC₅₀ of less than about 20 μM. In an even further aspect, the compound exhibits inhibition of viral hepatitis activity with an IC₅₀ of less than about 15 μM. In a still further aspect, the compound exhibits inhibition of viral hepatitis activity with an IC₅₀ of less than about 10 μM. In yet a further aspect, the compound exhibits inhibition of viral hepatitis activity with an IC₅₀ of less than about 5 μM. In an even further aspect, the compound exhibits inhibition of viral hepatitis activity with an IC₅₀ of less than about 1 μM. In a still further aspect, the compound exhibits inhibition of viral hepatitis activity with an IC₅₀ of less than about 0.5 μM.

In a further aspect, the compound exhibits inhibition of HSV activity with an IC₅₀ of less than about 30 μM. In a still further aspect, the compound exhibits inhibition of HSV activity with an IC₅₀ of less than about 25 μM. In yet a further aspect, the compound exhibits inhibition of HSV activity with an IC₅₀ of less than about 20 μM. In an even further aspect, the compound exhibits inhibition of HSV activity with an IC₅₀ of less than about 15 μM. In a still further aspect, the compound exhibits inhibition of HSV activity with an IC₅₀ of less than about 10 μM. In yet a further aspect, the compound exhibits inhibition of HSV with an IC₅₀ of less than about 5 μM. In an even further aspect, the compound exhibits inhibition of HSV activity with an IC₅₀ of less than about 1 μM. In a still further aspect, the compound exhibits inhibition HSV activity with an IC₅₀ of less than about 0.5 μM.

In a further aspect, the subject is a mammal. In a still further aspect, the subject is a human.

In a further aspect, the subject has been diagnosed with a need for treatment of the disorder prior to the administering step. In a still further aspect, the method further comprises the step of identifying a subject in need of treatment of the disorder.

3. Methods of Inhibiting a Viral Infection in at Least One Cell

In one aspect, disclosed are methods for inhibiting a viral infection in at least one cell, the method comprising the step of contacting the at least one cell with an effective amount of at least one disclosed compound, or a pharmaceutically acceptable salt thereof.

Thus, in one aspect, disclosed are methods for inhibiting a viral infection in at least one cell, the method comprising the step of contacting the at least one cell with an effective amount of at least one compound having a structure represented by a formula:

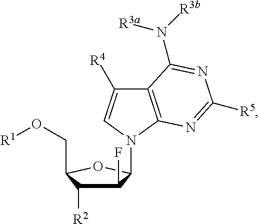

wherein R¹ is selected from hydrogen, —C(O)R¹⁰, —P(O)(OR¹¹)₂, and —P(O)(OR¹¹)R¹²; wherein R² is selected from hydrogen, —OH, C1-C8 alkoxy, —P(O)(OR^{11′})₂, and —P(O)(OR^{11′})R^{12′}; wherein R¹⁰, when present, is selected from C1-C30 alkyl, C2-C30 alkenyl, and —CH(NH₂)R²⁰; wherein R²⁰, when present, is selected from hydrogen, methyl, isopropyl, isobutyl, sec-butyl, —(CH₂)₃NHC(NH)NH₂, —(CH₂)₄NH₂, —CH₂CO₂H, —(CH₂)₂CO₂H, —CH₂OH, —CH(OH)CH₃, —CH₂C(O)NH₂, —(CH₂)₂C(O)NH₂, —CH₂SH, —(CH₂)₂SCH₃, —CH₂SeH, —CH₂C₆H₅, and -CH₂Cy¹; wherein Cy¹, when present, is selected from monocyclic aryl, para-hydroxy monocyclic aryl, 4-imidazolyl, and 3-indolyl; wherein each of R¹¹ and R^{11′}, when present, is independently selected from hydrogen, C1-C4 alkyl, —(C1-C10 alkyl)CO₂(C1-C10 alkyl), —(C1-C10 alkoxy)CO₂(C1-C10 alkyl), —(C1-C10 alkyl)CO₂(C1-C10 alkylthiol), —(C1-C10 alkyl)-S—S—(C1-C10 alkyl), Ar¹, and —CH₂Ar¹; wherein each occurrence of Ar¹, when present, is selected from aryl and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R¹² and R^{12′}, when present, is selected from —OR²¹ and —NHR²¹; wherein each occurrence of R²¹, when present, is selected from hydrogen, —(C1-C10 alkyl)CO₂(C1-C10 alkyl), —(C1-C10 alkoxy)CO₂(C1-C10 alkyl), —(C1-C10 alkyl)CO₂(C1-C10 alkylthiol), —(C1-C10 alkyl)-S—S—(C1-C10 alkyl), Ar², —CH₂Ar², —P(O)OHOP(O)(OH)₂, and a structure represented by a formula:

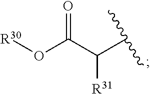

wherein each occurrence of R³⁰, when present, is independently selected from hydrogen, C1-C8 alkyl, Cy², and —CH₂Cy²; wherein each occurrence of Cy², when present, is independently selected from C3-C6 cycloalkyl, aryl, and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each occurrence of R³¹, when present, is independently selected from hydrogen and C1-C8 alkyl; and wherein each occurrence of Ar², when present, is independently selected from aryl and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; or wherein each of R¹ and R² together comprise a structure represented by a formula:

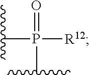

wherein each of R^{3a} and R^{3b} is independently selected from hydrogen, —OH, C1-C10 alkoxy, C1-C8 alkyl, —C(O)(C1-C30 alkyl), —C(O)(C2-C30 alkenyl), Cy³, —CR^{32a}R^{32b}Ar³; wherein each of R^{32a} and R^{32b}, when present, is independently selected from hydrogen and C1-C4 alkyl; wherein Cy³, when present, is C3-C6 cycloalkyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein Ar³, when present, is selected from aryl and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R⁴ is selected from hydrogen, halogen, —CN, —C(O)NH₂, —CO₂H, —COMe, —SO₂Me, C1-C4 haloalkyl, and Ar⁴; wherein Ar⁴, when present, is selected from aryl and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R⁵ is selected from halogen, —CF₃, C1-C10 alkyl, and Ar⁵; and wherein Ar⁵, when present, is selected from aryl and heteroaryl, and is substituted with 0, 1, 2, or 3 groups halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, or a pharmaceutically acceptable salt thereof.

In a further aspect, the cell is mammalian. In a still further aspect, the cell is human. In yet a further aspect, the cell has been isolated from a mammal prior to the contacting step.

In a further aspect, contacting is via administration to a mammal.

4. Use of Compounds

In one aspect, the invention relates to the use of a disclosed compound or a product of a disclosed method. In a further aspect, a use relates to the manufacture of a medicament for the treatment of a viral infection in a subject.

Also provided are the uses of the disclosed compounds and products. In one aspect, the invention relates to use of at least one disclosed compound; or a pharmaceutically acceptable salt, hydrate, solvate, or polymorph thereof. In a further aspect, the compound used is a product of a disclosed method of making.

In a further aspect, the use relates to a process for preparing a pharmaceutical composition comprising a therapeutically effective amount of a disclosed compound or a product of a disclosed method of making, or a pharmaceutically acceptable salt, solvate, or polymorph thereof, for use as a medicament.

In a further aspect, the use relates to a process for preparing a pharmaceutical composition comprising a therapeutically effective amount of a disclosed compound or a product of a disclosed method of making, or a pharmaceutically acceptable salt, solvate, or polymorph thereof, wherein a pharmaceutically acceptable carrier is intimately mixed with a therapeutically effective amount of the compound or the product of a disclosed method of making.

In various aspects, the use relates to a treatment of a viral infection in a subject. Also disclosed is the use of a compound for antagonism of a viral infection. In one aspect, the use is characterized in that the subject is a human. In one aspect, the use is characterized in that the disorder is a viral infection.

In a further aspect, the use relates to the manufacture of a medicament for the treatment of a viral infection in a subject.

In a further aspect, the use relates to antagonism of a viral infection in a subject. In a further aspect, the use relates to modulating viral activity in a subject. In a still further aspect, the use relates to modulating viral activity in a cell. In yet a further aspect, the subject is a human.

It is understood that the disclosed uses can be employed in connection with the disclosed compounds, products of disclosed methods of making, methods, compositions, and kits. In a further aspect, the invention relates to the use of a disclosed compound or a disclosed product in the manufacture of a medicament for the treatment of a viral infection in a mammal. In a further aspect, the viral infection is selected from human immunodeficiency virus (HIV), human papillomavirus (HPV), herpes simplex virus (HSV), human cytomegalovirus (HCMV), chicken pox, infectious mononucleosis, mumps, measles, rubella, shingles, ebola, viral gastroenteritis, viral hepatitis, viral meningitis, human metapneumovirus, human parainfluenza virus type 1, parainfluenza virus type 2, parainfluenza virus type 3, respiratory syncytial virus, viral pneumonia, Chikungunya virus (CHIKV), Venezuelan equine encephalitis (VEEV), dengue (DENV), influenza, West Nile virus (WNV), zika (ZIKV), 229E, NL63, OC43, HKU1, Middle East respiratory syndrome coronavirus (MERS-CoV), severe acute respiratory syndrome coronavirus (SARS-CoV), and severe acute respiratory syndrome coronavirus disease 2019 (SARS-CoV-2).

5. Manufacture of a Medicament

In one aspect, the invention relates to a method for the manufacture of a medicament for treating a viral infection in a subject having the viral infection, the method comprising combining a therapeutically effective amount of a disclosed compound or product of a disclosed method with a pharmaceutically acceptable carrier or diluent.

As regards these applications, the present method includes the administration to an animal, particularly a mammal, and more particularly a human, of a therapeutically effective amount of the compound effective in the inhibition of a viral infection. The dose administered to an animal, particularly a human, in the context of the present invention should be sufficient to affect a therapeutic response in the animal over a reasonable time frame. One skilled in the art will recognize that dosage will depend upon a variety of factors including the condition of the animal and the body weight of the animal.

The total amount of the compound of the present disclosure administered in a typical treatment is preferably between about 10 mg/kg and about 1000 mg/kg of body weight for mice, and between about 100 mg/kg and about 500 mg/kg of body weight, and more preferably between 200 mg/kg and about 400 mg/kg of body weight for humans per daily dose. This total amount is typically, but not necessarily, administered as a series of smaller doses over a period of about one time per day to about three times per day for about 24 months, and preferably over a period of twice per day for about 12 months.

The size of the dose also will be determined by the route, timing and frequency of administration as well as the existence, nature and extent of any adverse side effects that might accompany the administration of the compound and the desired physiological effect. It will be appreciated by one of skill in the art that various conditions or disease states, in particular chronic conditions or disease states, may require prolonged treatment involving multiple administrations.

Thus, in one aspect, the invention relates to the manufacture of a medicament comprising combining a disclosed compound or a product of a disclosed method of making, or a pharmaceutically acceptable salt, solvate, or polymorph thereof, with a pharmaceutically acceptable carrier or diluent.

6. Kits

In one aspect, disclosed are kits comprising at least one disclosed compound and one or more of: (a) at least one antiviral agent; (b) a instructions for administering the at least one compound in connection with treating a viral infection; (c) instructions for administering the at least one compound in connection with reducing the risk of viral infection; and (d) instructions for treating a viral infection.

In a further aspect, disclosed are kits comprising at least one compound having a structure represented by a formula:

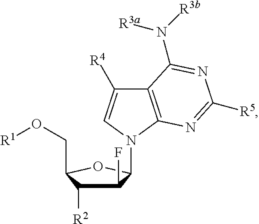

wherein R¹ is selected from hydrogen, —C(O)R¹⁰, —P(O)(OR¹¹)₂, and —P(O)(OR¹¹)R¹²; wherein R² is selected from hydrogen, —OH, C1-C8 alkoxy, —P(O)(OR^{11′})₂, and —P(O)(OR^{11′})R^{12′}; wherein R¹⁰, when present, is selected from C1-C30 alkyl, C2-C30 alkenyl, and —CH(NH₂)R²⁰; wherein R²⁰, when present, is selected from hydrogen, methyl, isopropyl, isobutyl, sec-butyl, —(CH₂)₃NHC(NH)NH₂, —(CH₂)₄NH₂, —CH₂CO₂H, —(CH₂)₂CO₂H, —CH₂OH, —CH(OH)CH₃, —CH₂C(O)NH₂, —(CH₂)₂C(O)NH₂, —CH₂SH, —(CH₂)₂SCH₃, —CH₂SeH, —CH₂C₆H₅, and —CH₂Cy¹; wherein Cy¹, when present, is selected from monocyclic aryl, para-hydroxy monocyclic aryl, 4-imidazolyl, and 3-indolyl; wherein each of R¹¹ and R^{11′}, when present, is independently selected from hydrogen, C1-C4 alkyl, —(C1-C10 alkyl)CO₂(C1-C10 alkyl), —(C1-C10 alkoxy)CO₂(C1-C10 alkyl), —(C1-C10 alkyl)CO₂(C1-C10 alkylthiol), —(C1-C10 alkyl)-S—S—(C1-C10 alkyl), Ar¹, and —CH₂Ar¹; wherein each occurrence of Ar¹, when present, is selected from aryl and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R¹² and R^{12′}, when present, is selected from —OR²¹ and —NHR²¹; wherein each occurrence of R²¹, when present, is selected from hydrogen, —(C1-C10 alkyl)CO₂(C1-C10 alkyl), —(C1-C10 alkoxy)CO₂(C1-C10 alkyl), —(C1-C10 alkyl)CO₂(C1-C10 alkylthiol), —(C1-C10 alkyl)-S—S—(C1-C10 alkyl), Ar², —CH₂Ar², —P(O)OHOP(O)(OH)₂, and a structure represented by a formula:

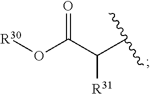

wherein each occurrence of R³⁰, when present, is independently selected from hydrogen, C1-C8 alkyl, Cy², and —CH₂Cy²; wherein each occurrence of Cy², when present, is independently selected from C3-C6 cycloalkyl, aryl, and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each occurrence of R³¹, when present, is independently selected from hydrogen and C1-C8 alkyl; and wherein each occurrence of Ar², when present, is independently selected from aryl and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; or wherein each of R¹ and R² together comprise a structure represented by a formula:

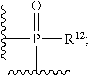

wherein each of R^{3a} and R^{3b} is independently selected from hydrogen, —OH, C1-C10 alkoxy, C1-C8 alkyl, —C(O)(C1-C30 alkyl), —C(O)(C2-C30 alkenyl), Cy³, —CR^{32a}R^{32b}Ar³; wherein each of R^{32a} and R^{32b}, when present, is independently selected from hydrogen and C1-C4 alkyl; wherein Cy³, when present, is C3-C6 cycloalkyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein Ar³, when present, is selected from aryl and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R⁴ is selected from hydrogen, halogen, —CN, —C(O)NH₂, —CO₂H, —COMe, —SO₂Me, C1-C4 haloalkyl, and Ar⁴; wherein Ar⁴, when present, is selected from aryl and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R⁵ is selected from halogen, —CF₃, C1-C10 alkyl, and Ar⁵; and wherein Ar⁵, when present, is selected from aryl and heteroaryl, and is substituted with 0, 1, 2, or 3 groups halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, or a pharmaceutically acceptable salt thereof, and one or more of: (a) at least one antiviral agent; (b) a instructions for administering the at least one compound in connection with treating a viral infection; (c) instructions for administering the at least one compound in connection with reducing the risk of viral infection; and (d) instructions for treating a viral infection.

In a further aspect, the viral infection is selected from human immunodeficiency virus (HIV), human papillomavirus (HPV), herpes simplex virus (HSV), human cytomegalovirus (HCMV), chicken pox, infectious mononucleosis, mumps, measles, rubella, shingles, ebola, viral gastroenteritis, viral hepatitis, viral meningitis, human metapneumovirus, human parainfluenza virus type 1, parainfluenza virus type 2, parainfluenza virus type 3, respiratory syncytial virus, viral pneumonia, Chikungunya virus (CHIKV), Venezuelan equine encephalitis (VEEV), dengue (DENV), influenza, West Nile virus (WNV), zika (ZIKV), 229E, NL63, OC43, HKU1, Middle East respiratory syndrome coronavirus (MERS-CoV), severe acute respiratory syndrome coronavirus (SARS-CoV), and severe acute respiratory syndrome coronavirus disease 2019 (SARS-CoV-2). In a still further aspect, the viral infection is viral hepatitis. In yet a further aspect, the viral hepatitis is hepatitis B virus (HBV). In an even further aspect, the viral hepatitis is herpes simplex virus.

In a still further aspect, the antiviral agent is selected from selected from acemannan, acyclovir, acyclovir sodium, adamantanamine, adefovir, adenine arabinoside, alovudine, alvircept sudotox, amantadine hydrochloride, aranotin, arildone, atevirdine mesylate, avridine, cidofovir, cipamfylline, cytarabine hydrochloride, BMS 806, C31G, carrageenan, cellulose sulfate, cyclodextrins, dapivirine, delavirdine mesylate, desciclovir, dextrin 2-sulfate, didanosine, disoxaril, dolutegravir, edoxudine, enviradene, envirozime, etravirine, famciclovir, famotine hydrochloride, fiacitabine, fialuridine, fosarilate, foscarnet sodium, fosfonet sodium, FTC, ganciclovir, ganciclovir sodium, GSK 1265744, 9-2-hydroxy-ethoxy methylguanine, ibalizumab, idoxuridine, interferon, 5-iodo-2′-deoxyuridine, IQP-0528, kethoxal, lamivudine, lobucavir, maraviroc, memotine pirodavir, penciclovir, raltegravir, ribavirin, rimantadine hydrochloride, rilpivirine (TMC-278), saquinavir mesylate, SCH-C, SCH-D, somantadine hydrochloride, sorivudine, statolon, stavudine, T20, tilorone hydrochloride, TMC120, TMC125, trifluridine, trifluorothymidine, tenofovir, tenofovir alefenamide, tenofovir disoproxyl fumarate, prodrugs of tenofovir, UC-781, UK-427, UK-857, valacyclovir, valacyclovir hydrochloride, vidarabine, vidarabine phosphate, vidarabine sodium phosphate, viroxime, zalcitabene, zidovudine, and zinviroxime.

In a further aspect, the at least one compound and the at least one agent are co-formulated. In a further aspect, the at least one compound and the at least one agent are co-packaged.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is understood that the disclosed kits can be prepared from the disclosed compounds, products, and pharmaceutical compositions. It is also understood that the disclosed kits can be employed in connection with the disclosed methods of using.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

### F. Examples

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The Examples are provided herein to illustrate the invention, and should not be construed as limiting the invention in any way. Examples are provided herein to illustrate the invention and should not be construed as limiting the invention in any way.

### 1. Chemistry Experimentals

### A. General Experimental

The reactions were performed under a dry argon atmosphere and reaction temperatures were measured externally. Anhydrous solvents over molecular sieves were purchased from Aldrich and used as such in reactions. Microwave (MW) reactions were performed in CEM Discover Labmate System with Intelligent Technology for Focused™ Microwave Synthesizer (Explorer 48) or Biotage Initiator+ equipped with Robot Eight microwave system. The reactions were monitored by thin-layer chromatography (TLC) on pre-coated silica gel (60F₂₅₄) aluminium plates (0.25 mm) from E. Merck and visualized using UV light (254 nm). Purification of compounds was performed on an Isco Teledyne Combiflash Rf200. Universal RediSep solid sample loading pre-packed cartridges (5.0 g silica) were used to absorb crude product and purified on 12 g silica RediSep Rf Gold Silica (20-40 μm spherical silica) columns using appropriate solvent gradients. Pure samples were dried overnight under high vacuum before analyses. The high resolution electrospray ionization mass spectral data (HR-ESIMS) were obtained on an Agilent LC-MSTOF. ¹H NMR spectra were recorded at 400 MHz on Agilent/Varian MR-400 spectrometer in CDCl₃, CD₃OD, or DMSO-d₆ as solvents. The chemical shifts (6) are in ppm downfield from standard tetramethylsilane (TMS). HPLC of final compounds were run on an Agilent 1100 LC equipped with a diode array UV detector and were monitored at 254 nm using the following using Sunfire C18 column (5 μm, 4.6×150 mm) using H₂O—CH₃CN (both containing 0.1% formic acid) 5-95% in 20 min with flow rate 1.0 mL/min.

### b. Procedure for the Synthesis of 7-deaza-2′-deoxy-2′fluoroarabinosyl Nucleoside Analogs

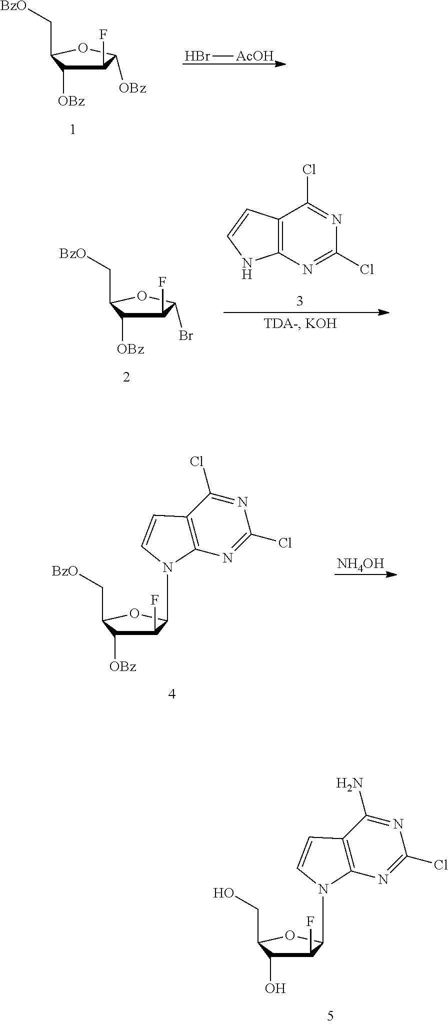

### i. Preparation of ((2R,3R,4S,5R)-3-(benzoyloxy)-5-bromo-4-fluorotetrahydrofuran-2-yl)methyl benzoate (2)

To a cold (−5° C.) solution of (2R,3S,4R,5R)-5-((benzoyloxy)-methyl)-3-fluorotetrahydrofuran-2,4-diyl-dibenzoate 1 (30.0 g, 64.59 mmol, 1.0 eq) in anhydrous dichloromethane (140 mL) was added 33% hydrobromic acid (35.1 mL, 193.78 mmol, 3.0 eq) in acetic acid, dropwise, over 20 min. Upon completion of addition, the reaction mixture was stirred for 18 hrs as it warmed to 20° C. The reaction mixture was evaporated under reduced pressure to afford a red oil, which was dissolved in dichloromethane (300 mL) and then washed with water (3×100 mL), sat NaHCO₃ (2×100 mL), followed by brine (100 mL). The organic layer was separated, dried (Na₂SO₄), filtered, and then the filtrate was evaporated in vacuo to provide 27.68 g (100%) of 2 as a light brown oil. ¹H NMR (CDCl₃) δ 8.16-8.04 (m, 4H), 7.68-7.54 (m, 2H), 7.54-7.40 (m, 4H), 6.65 (dt, J=12.2, 1.0 Hz, 1H), 5.71-5.50 (m, 2H), 4.88-4.67 (m, 3H); ¹⁹F NMR δ_{F}-165.86 to −166.11 (m, 1F).

### ii. Preparation of ((2R,3R,4S,5R)-3-(benzoyloxy)-5-(2,4-dichloro-7H-pyrrolo[2,3-d]pyrimidin-7-yl)-4-fluorotetrahydrofuran-2-yl)methyl benzoate (4)

To a mixture of anhydrous acetonitrile (300 mL) and potassium hydroxide (4.92 g, 87.6 mmol, 2.12 eq) was added catalytic Tris[2-(2-methoxyethoxy)ethyl]amine (TDA-1) (0.793 mL, 2.48 mmol, 0.06 eq). The mixture was stirred for 20 min and then the nucleobase 3 (7.77 g, 41.3 mmol, 1.0 eq) was added. The reaction mixture was stirred for 30 min after which time a solution of the brominated sugar 2 (20.99 g, 49.59 mmol, 1.2 eq) in anhydrous acetonitrile (200 mL) was added. The reaction mixture was stirred at 20° C. for 18 hrs. The reaction mixture was quenched with sat NH₄Cl (300 mL). The organic layer was separated and then evaporated in vacuo to afford a tacky solid, which was suspended into the aqueous layer above and then extracted with dichloromethane (3×100 mL). The organic extracts were combined and washed with brine (100 mL). The organic layer was separated, dried (Na₂SO₄), filtered, and then the filtrate was evaporated under reduced pressure to give 27.78 g of a crude tan tacky solid. Purification by flash chromatography (5×120 g silica columns, 100-70% hexane in ethyl acetate, gradient elution) provided 14.4 g (66%) of 4 as a whited foamy solid. ¹H NMR (CDCl₃) δ 8.17-8.08 (m, 4H), 7.73-7.39 (m, 7H), 6.80 (dd, J=22.3, 2.9 Hz, 1H), 6.66 (d, J=3.8 Hz, 1H), 5.76 (ddd, J=17.7, 3.1, 0.9 Hz, 1H), 5.36 (ddd, J=50.1, 3.0, 0.8 Hz, 1H), 4.87-4.76 (m, 2H), 4.57 (td, J=4.6, 3.0 Hz, 1H); ¹⁹F NMR δ_{F}−198.27 to −198.52 (m, 1F); LCMS m/z 530 (M+H)⁺.

### iii. Synthesis of (2R,3R,4S,5R)-5-(4-amino-2-chloro-7H-pyrrolo[2,3-d]pyrimidin-7-yl)-4-fluoro-2-(hydroxymethyl)tetrahydrofuran-3-ol (5)

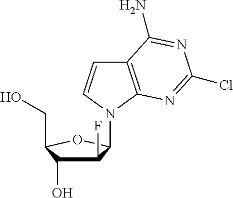

To a steel bomb was added the nucleoside 4 (1.39 g, 2.62 mmol, 1.0 eq), 1,4-dioxane (5.0 mL), followed by 28% aqueous ammonium hydroxide (5.10 mL, 38.01 mmol, 14.5 eq). The reaction mixture was stirred at 80° C. for 18 hrs. The reaction mixture was evaporated at 40° C. under reduced pressure to afford a semi-solid, which was purified by flash chromatography (40 g silica column, 100-90% dichloromethane in methanol, gradient elution) to provide 669 mg (84%) of 5 as a white powder. ¹H NMR (DMSO-d6) δ 7.60 (s, 2H), 7.29 (dd, J=3.7, 2.3 Hz, 1H), 6.64 (dd, J=3.7, 0.4 Hz, 1H), 6.43 (dd, J=15.7, 4.4 Hz, 1H), 5.91 (d, J=5.2, 1H), 5.23-4.96 (m, 2H), 4.36 (dtd, J=18.9, 5.2, 3.7 Hz, 1H), 3.86-3.76 (m, 1H), 3.73-3.54 (m, 2H); ¹⁹F NMR δ_{F}-198.50 to −198.73 (m, 1F); LCMS m/z 303 (M+H)⁺; HRMS m/z 303.0655 (M+H)⁺; HPLC 96.9% at 254 nm.

### c. Procedure for the Synthesis of 7-Deaza-2′-deoxy-2′-fluoroarabinosyl Nucleotide Analogs

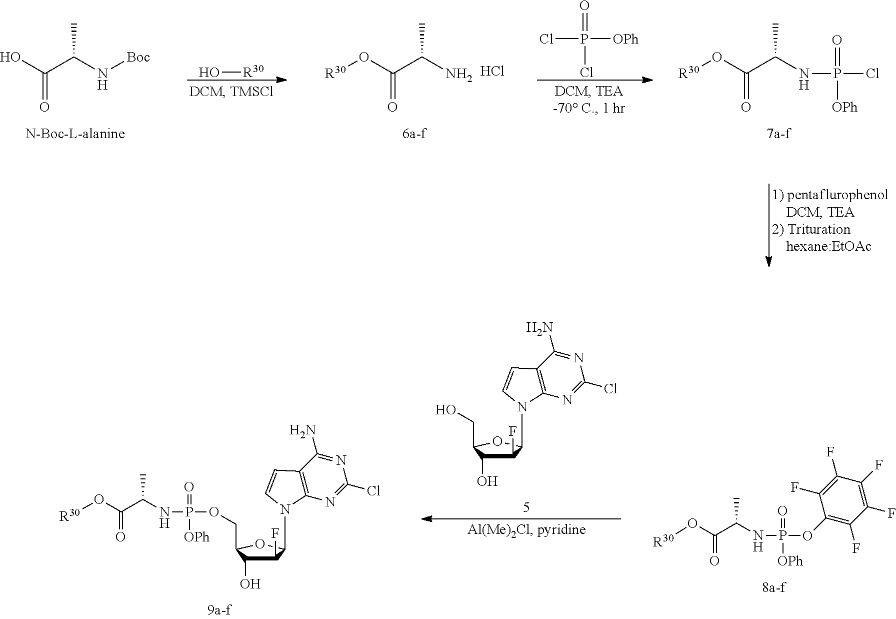

### i. Synthesis of 2-ethylbutyl ((((2R,3R,4S,5R)-5-(4-amino-2-chloro-7H-pyrrolo[2,3-d]pyrimidin-7-yl)-4-fluoro-3-hydroxytetrahydrofuran-2-yl)methoxy)(phenoxy)phosphoryl)-L-alaninate (9a)

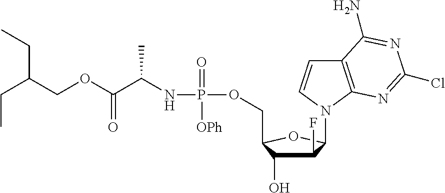

### (i) Preparation of 2-ethylbutyl L-alaninate hydrochloride (6a)

To a solution of N-Boc-L-alinine (10.0 g, 52.85 mmol, 1.0 Eq.) in 2-ethyl-1-butanol (100 mL, 15.5 Eq.) was added trimethylsilyl chloride (33.5 mL, 264 mmol, 5.0 eq). The reaction mixture was stirred at 20° C. for 18 hrs under argon. The reaction mixture was evaporated under reduced pressure at 40-60° C. to afford a semi-solid, which was triturated in 100 mL of anhydrous diethyl ether for 18 hrs under argon. The mixture was filtered by vacuum filtration to collect a solid which was rinsed with anhydrous diethyl ether (2×20 mL) dried under reduced pressure at 40° C. to provide 9.40 g (85%) of 6a as a white solid. ¹H-NMR (DMSO-d₆) δ 8.59 (s, 3H), 4.18-4.01 (m, 3H), 1.53 (hept, J=6.1 Hz, 1H), 1.44 (d, J=7.2 Hz, 3H), 1.41-1.29 (m, 4H), 0.88 (t, J=7.4 Hz, 6H).

### (ii) Preparation 2-ethylbutyl ((S)-(perfluorophenoxy)(phenoxy)phosphoryl)-L-alaninate (8a)

To a mixture of 6a (5.0 g, 23.84 mmol, 1.0 eq) in 70 mL of anhydrous dichloromethane was added phenyl phosphorodichloridate (3.91 mL, 26.5 mmol, 1.1 eq). The mixture was cooled to −72° C. and then a solution of triethyl amine (6.9 mL, 50 mmol, 2.1 eq) in 30 mL of anhydrous dichloromethane was added over 2 hrs and 20 min at −70° C. Upon completion of addition, the reaction mixture was stirred at −72° C. for 2 hrs and then for 18 hrs as it warmed to 20° C. The reaction mixture was evaporated under reduced pressure to afford a semi-solid, which was triturated in 50 mL of anhydrous t-butyl methyl ether for 1 hr under argon. The mixture was filtered by vacuum filtration to remove triethyl amine hydrochloride, which was rinsed with anhydrous t-butyl methyl ether (2×50 mL). The filtrate was evaporated in vacuo to provide 8.82 g of 7a as a colorless oil which was used as is without further purification.

To a cold (−5° C.) solution of 7a (8.3 g, 23.84 mmol, 1.0 eq) in 60 mL of anhydrous dichloromethane was added a solution of pentafluorophenol (4.82 g, 26.22 mmol, 1.1 eq) and triethylamine (3.65 mL, 26.22 mmol, 1.1 eq) in 25 mL of anhydrous dichloromethane over 1 hr at −5° C. The reaction mixture was stirred at 0° C. for 2 hrs and then for 18 hrs as it warmed to 20° C. The reaction mixture was evaporated under reduced pressure to afford a semi-solid, which was triturated in ethyl acetate (100 mL) and then stirred for 30 min. The mixture was filtered by vacuum filtration to remove triethylamine hydrochloride. The filtrate was washed with water (2×500 mL), 10% Na₂CO₃ (2×100 mL), NH₄Cl (100 mL), followed by brine (25 mL). The organic layer was separated, dried (Na₂SO₄), filtered, and then the filtrate was evaporated in vacuo to give 16.0 g of a crude semi-solid. The material was purified in two portions by flash chromatography (220 g column, 100 to 70% hexane in ethyl acetate, gradient elution) to provide a combined mass of 8.0 g of a solid. Trituration from 95% hexane in ethyl acetate (100 mL) gave 4.7 g (41%) of 8a as white needles and as a single diastereomer. ¹H-NMR (DMSO-d6) δ 7.48-7.38 (m, 2H), 7.30-7.19 (m, 3H), 6.90 (dd, J=14.2, 9.9 Hz, 1H), 4.12-3.88 (m, 3H), 1.46 (h, J=6.1 Hz, 1H), 1.37-1.22 (m, 7H), 0.84 (t, J=7.5 Hz, 6H); ¹⁹F-NMR δ_{F}-153.25 to −154.25 (m, 2F), −160.38 (td, J=23.6, 3.3 Hz, 1F), −163.07 (td, J=23.6, 4.1 Hz, 2F); ³¹P-NMR δ_{P} 0.26; LCMS: m/z 496 (M+H)⁺.

### (iii) Preparation of 9a

To an oven dried 50 mL rbf was added the nucleoside 5 (105 mg, 0.330 mmol, 1.0 eq). Anhydrous pyridine (5.0 mL) was added and then evaporated under reduced pressure at 30° C. to remove residual water. This was done one more time with a fresh portion of pyridine (5.0 mL). The nucleoside was dissolved in anhydrous pyridine (1.50 mL) and then the phosphoramidate 8a (196 mg, 0.396 mmol, 1.2 eq) was added. The solution was cooled to −5° C. and then dimethyl aluminum chloride (0.165 mL, 0.165 mmol, 1.0 eq) was added all at once. Upon completion of addition, the reaction mixture was stirred under 0° C. for 2 hrs and then for 20 hrs as it warmed to 20° C. The reaction mixture was evaporated in vacuo to afford an oil, which was purified by flash chromatography (40 g silica column, 100-95% dichloromethane in methanol, gradient elution) to provide 39 mg (18%) of 9a as a white foamy solid and as a single diastereomer (S,Sp). ¹H NMR (DMSO-d6) δ 7.61 (s, 2H), 7.43-7.33 (m, 2H), 7.27-7.14 (m, 4H), 6.63 (dd, J=3.7, 0.4 Hz, 1H), 6.48 (dd, J=16.9, 4.3 Hz, 1H), 6.13-6.00 (m, 2H), 5.24-5.09 (m, 1H), 4.46-4.33 (m, 1H), 4.32-4.12 (m, 2H), 4.04-3.80 (m, 4H), 1.44 (hept, J=6.1 Hz, 1H), 1.34-1.12 (m, 7H), 0.82 (t, J=7.4 Hz, 6H); ³¹P NMR δ_{P}3.64; ¹⁹F NMR δ_{F}-198.34 to −198.58 (m, 1F); LCMS m/z 614 (M+H)⁺; HRMS m/z 614.1935 (M+H)⁺; HPLC 97.1% at 254 nm.

### ii. Synthesis of isopropyl ((((2R,3R,4S,5R)-5-(4-amino-2-chloro-7H-pyrrolo[2,3-d]pyrimidin-7-yl)-4-fluoro-3-hydroxytetrahydrofuran-2-yl)methoxy)(phenoxy)phosphoryl)-L-alaninate (9b)

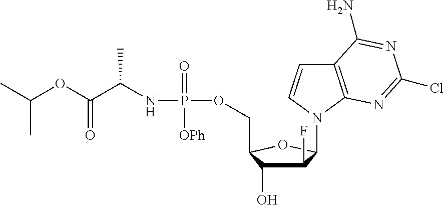

### (i) Preparation of isopropyl ((S)-(perfluorophenoxy)(phenoxy)phosphoryl)-L-alaninate (8b)

To a mixture of isopropyl-L-alaninate 6b (2.0 g, 11.93 mmoles, 1.0 eq) in 20 mL of anhydrous dichloromethane was added phenyl phosphorodichloridate (1.96 mL, 13.12 mmoles, 1.1 eq). The mixture was cooled to −70° C. and then a solution of triethylamine (3.49 mL, 25.05 mmoles, 2.1 eq) in 10 mL of anhydrous dichloromethane was added over 1 hr and 10 min at −70° C. Upon completion of addition, the reaction mixture was stirred at −70° C. for 1 hr and then for 18 hrs as it warmed to 20° C. The reaction mixture was evaporated under reduced pressure to afford a solid, which was triturated in 50 mL of anhydrous t-butyl methyl ether for 2 hrs. The mixture was filtered by vacuum filtration to remove triethyl amine hydrochloride, which was rinsed with anhydrous t-butyl methyl ether (2×20 mL). The filtrate was evaporated in vacuo to provide 3.65 g of isopropyl 7b as a colorless oil.

To a cold (−5° C.) solution of 7b (3.65 g, 11.93 mmoles, 1.0 eq) in 20 mL of anhydrous dichloromethane was added a solution of pentafluorophenol (2.41 g, 13.12 mmoles, 1.1 eq) and trimethylamine (1.83 mL, 13.12 mmoles, 1.1 eq) in 10.0 mL of anhydrous dichloromethane over 20 min at −5° C. The reaction mixture was stirred for 2 hrs at −5° C. and then for 18 hrs as it warmed to 20° C. The reaction mixture was evaporated under reduced pressure to afford a solid, which was suspended in ethyl acetate (100 mL) and then stirred for 30 min. The mixture was filtered by vacuum filtration to remove triethylamine hydrochloride. The filtrate was washed with water (2×50 mL), 10% Na₂CO₃ (2×50 mL), followed by brine (100 mL). The organic layer was separated, dried (Na₂SO₄), filtered, and then the filtrate was evaporated in vacuo to give a crude white solid. Purification by flash chromatography (120 g column, 100% to 70% hexane in ethyl acetate, gradient elution), followed by trituration in 95% hexane in ethyl acetate hexane (30 mL) provided 2.22 g (41%) of 8b as a white solid and as a single diastereomer. ¹H-NMr (DMSO-d6) δ 7.48-7.39 (m, 2H), 7.32-7.20 (m, 3H), 6.99-6.74 (m, 1H), 4.89 (pd, J=6.3, 5.5 Hz, 1H), 4.02-3.82 (m, 1H), 1.29 (ddd, J=7.1, 4.6, 1.2 Hz, 3H), 1.17 (dd, J=6.3, 1.1 Hz, 6H); ¹⁹F-NMR δ_{F}-153.76 (t, J=21.2 Hz, 2F), −159.94 to −160.90 (m, 1F), −162.68 to −163.68 (m, 2F); ³¹P-NMR δ_{P} 0.31; LCMS: m/z 454 (M+H)⁺.

### (ii) Preparation of 9b

The final product 9b was prepared from 5 (88 mg, 0.291 mmoles, 1.0 eq) and 8b (158 mg, 0.349 mmoles, 1.2 eq) according to the procedure described for the preparation of 9a. Purification by flash chromatography (40 g silica column, 100-95% dichloromethane in methanol, gradient elution) provided 51 mg (310%) as a white foamy solid and as a mixture of two diastereomers (2:1). ¹H NMR (DMSO-d6) δ 7.61 (s, 2H), 7.42-7.32 (m, 2H), 7.26-7.14 (m, 4H), 6.68-6.59 (m, 1H), 6.48 (ddd, J=17.0, 7.3, 4.4 Hz, 1H), 6.13-5.97 (m, 2H), 5.25-5.09 (m, 1H), 4.86 (pd, J=6.3, 5.3 Hz, 1H), 4.40 (dq, J=18.7, 4.6 Hz, 1H), 4.33-4.12 (m, 2H), 4.08-3.70 (m, 2H), 1.26-1.18 (m, 3H), 1.18-1.12 (m, 6H); ³¹P NMR δ_{P} 3.68, 3.61; ¹⁹F NMR δ_{F}−198.30 to −198.53 (m, 1F); LCMS m/z 572 (M+H)⁺; HRMS m/z 572.147 (M+H)⁺; HPLC 96.5% at 254 nm.

### iii. Synthesis of benzyl ((((2R,3R,4S,5R)-5-(4-amino-2-chloro-7H-pyrrolo[2,3-d]pyrimidin-7-yl)-4-fluoro-3-hydroxytetrahydro-furan-2-yl)methoxy)-(phenoxy)phosphoryl)-L-alaninateate (9c)

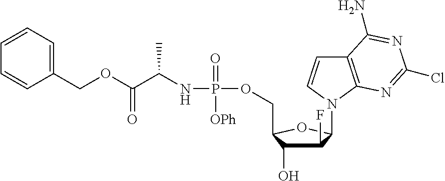

### (i) Preparation of benzyl ((perfluorophenoxy)-(phenoxy)-phosphoryl)-L-alaninate (8c)

Intermediate 7c was prepared from commercial benzyl-L-alaninate hydrochloride 6c (10.0 g, 46.37 mmoles, 1.0 eq) and phenyl phosphorodichloridate (7.60 mL, 51.0 mmoles, 1.1 eq) in 140 mL of anhydrous dichloromethane with triethylamine (13.57 mL, 97.37 mmoles, 2.1 eq) as base according to the procedure described for the preparation of 7b to afford 18.02 g of a yellow-green oil. Intermediate 8c was prepared from 7c (16.4 g, 46.37 mmoles, 1.0 eq) and pentafluorophenol (9.39 g, 51.0 mmoles, 1.1 eq) in 120 mL of anhydrous dichloromethane with triethylamine (7.11 mL, 51.0 mmoles, 1.1 eq) as base according to the procedure described for the preparation of 8b to afford 11.24 g (48%) of a white solid and as a single diastereomer. ¹H NMR (400 MHz, DMSO-d₆) δ 7.44-7.30 (m, 7H), 7.29-7.19 (m, 3H), 6.97 (dd, J=14.1, 9.9 Hz, 1H), 5.12 (s, 2H), 4.17-3.94 (m, 1H), 1.33 (dd, J=7.1, 1.3 Hz, 3H); ¹⁹F NMR (376 MHz, DMSO-d₆) δ_{F} δ−153.30-−154.12 (m, 2F), −160.26 (td, J=23.6, 3.5 Hz, 1F), −163.14 (td, J=23.6, 4.1 Hz, 2F); ³¹P NMR δ_{P} 0.26; LCMS m/z 502 (M+H)⁺.

### (ii) Preparation of 9c

The final target 9c was prepared from 5 (100 mg, 0.330 mmoles, 1.0 eq), 8c (199 mg, 0.396 mmoles, 1.2 eq), and a 1M (in hexanes) dimethylaluminum chloride (0.165 mL, 0.165 mmoles, 0.50 eq) in 1.0 mL of anhydrous pyridine according to the procedure described for the preparation of 9a to afford, after purification by flash chromatography (40 g silica column, 100-92% dichloromethane in methanol, gradient elution), 17 mg (8%) of a white solid as a single diastereomer. 1H NMR (400 MHz, DMSO-d6) δ 7.61 (s, 2H), 7.40-7.29 (m, 7H), 7.25-7.14 (m, 4H), 6.63 (dd, J=3.7, 0.4 Hz, 1H), 6.48 (dd, J=16.9, 4.3 Hz, 1H), 6.15 (dd, J=13.1, 10.0 Hz, 1H), 6.08 (d, J=5.1 Hz, 1H), 5.25-5.02 (m, 3H), 4.47-4.34 (m, 1H), 4.32-4.10 (m, 2H), 4.05-3.87 (m, 2H), 1.27 (dd, J=7.1, 1.0 Hz, 3H); 19F NMR (376 MHz, DMSO-d6) δF −198.29 to −198.53 (m, 1F); 31P NMR δP 3.66; LCMS m/z 620 (M+H)+; HRMS calc for C27H28ClFN5O7P.H, 620.14717, found, 620.14714; HPLC 91.6% at 254 nm.

### iv. Synthesis of isobutyl ((((2R,3R,4S,5R)-5-(4-amino-2-chloro-7H-pyrrolo[2,3-d]pyrimidin-7-yl)-4-fluoro-3-hydroxytetrahydro-furan-2-yl)methoxy)-(phenoxy)phosphoryl)-L-alaninate (9d)

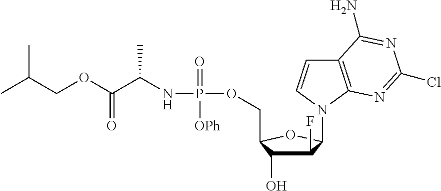

### (i) Preparation of isobutyl-L-alinate hydrochloride (6d)

Intermediate 6d was prepared from N-Boc-L-alinine (3.0 g, 15.86 mmoles, 1.0 eq) and chlorotrimethylsilane (10.0 mL, 79.28 mmoles, 5.0 eq) in 100 mL of 2-methyl-1-propanol (69 eq) according to the procedure described for the preparation of 6a to afford 2.66 g (92%) of a white solid. ¹H NMR (400 MHz, DMSO-d₆) δ 8.61 (s, 3H), 4.09 (q, J=7.2 Hz, 1H), 4.04-3.89 (m, 2H), 1.94 (dh, J=13.4, 6.6 Hz, 1H), 1.45 (d, J=7.2 Hz, 3H), 0.93 (dd, J=6.7, 0.7 Hz, 6H); LCMS m/z 145 (M−HCl)⁺.

### (ii) Preparation of isobutyl-((perfluorophenoxy)-(phenoxy)-phosphoryl)-L-alaninate (8d)

Intermediate 7d was prepared from 6d (2.0 g, 11.01 mmoles, 1.0 eq) and phenyl phosphorodichloridate (1.81 mL, 12.11 mmoles, 1.1 eq) in 20 mL of anhydrous dichloromethane with triethylamine (3.22 mL, 23.12 mmoles, 2.1 eq) as base according to the procedure described for the preparation of 7b to afford 3.83 g of a colorless oil. Intermediate 8d was prepared from 7d (3.83 g, 11.98 mmoles, 1.0 eq) and pentafluorophenol (2.43 g, 13.18 mmoles, 1.1 eq) in 20 mL of anhydrous dichloromethane with triethylamine (1.84 mL, 13.18 mmoles, 1.1 eq) as base according to the procedure described for the preparation of 8b to afford 1.43 g (26%) of a white solid and as a single diastereomer. 1H NMR (400 MHz, DMSO-d6) δ 7.48-7.38 (m, 2H), 7.30-7.19 (m, 3H), 6.90 (dd, J=14.1, 9.9 Hz, 1H), 4.11-3.94 (m, 1H), 3.84 (dd, J=6.6, 0.6 Hz, 2H), 1.93-1.79 (m, J=6.7 Hz, 1H), 1.32 (dd, J=7.1, 1.2 Hz, 3H), 0.88 (d, J=6.7 Hz, 6H); 31P NMR δP 0.29; LCMS m/z 468 (M+H)⁺.

### (iii) Preparation of 9d

The final target 9d was prepared from 5 (100 mg, 0.330 mmoles, 1.0 eq), 8d (185 mg, 0.396 mmoles, 1.2 eq), and a 1M (in hexanes) dimethylaluminum chloride (0.165 mL, 0.165 mmoles, 0.50 eq) in 1.5 mL of anhydrous pyridine according to the procedure described for the preparation of 9a to afford a residue. Purification by flash chromatography (40 g silica column, 100-90% dichloromethane in methanol, gradient elution), provided 45 mg (23%) of a white foamy solid as a single diastereomer. ¹H NMR (400 MHz, DMSO-d₆) δ 7.57 (s, 2H), 7.39-7.29 (m, 2H), 7.23-7.11 (m, 4H), 6.59 (d, J=3.7 Hz, 1H), 6.44 (dd, J=16.8, 4.3 Hz, 1H), 6.09-5.96 (m, 2H), 4.36 (dq, J=18.8, 4.8 Hz, 1H), 4.28-4.09 (m, 2H), 3.98 (q, J=3.5 Hz, 1H), 3.90-3.66 (m, 3H), 1.79 (dt, J=13.4, 6.7 Hz, 1H), 1.22 (dd, J=7.1, 0.9 Hz, 3H), 0.81 (dd, J=6.7, 1.7 Hz, 6H); ¹⁹F NMR (376 MHz, DMSO-d₆) δ_{F}−198.33 to −198.56 (m, 1F); ³¹P NMR δ_{P} 3.64; LCMS m/z 586 (M+H)⁺; HRMS calc for C₂₄H₃₀ClFN₅O₇P.H, 586.16252, found, 586.16188; HPLC 93.8% at 254 nm.

### v. Synthesis of ethyl ((((2R,3R,4S,5R)-5-(4-amino-2-chloro-7H-pyrrolo[2,3-d]pyrimidin-7-yl)-4-fluoro-3-hydroxytetrahydro-furan-2-yl)methoxy)-(phenoxy)phosphoryl)-L-alaninate (9e)

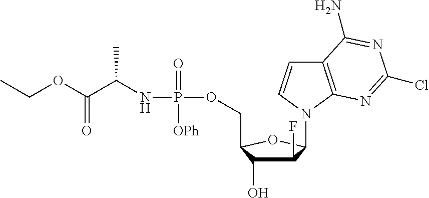

### (i) Preparation of ethyl((perfluorophenoxy)-(phenoxy)-phosphoryl)-L-alaninate (8e)

Intermediate 7e was prepared from commercial ethyl-L-alaninate hydrochloride 6e (6.50 g, 42.34 mmoles, 1.0 eq) and phenyl phosphorodichloridate (6.94 mL, 46.58 mmoles, 1.1 eq) in 70 mL of anhydrous dichloromethane with triethylamine (12.13 mL, 88.92 mmoles, 2.1 eq) as base according to the procedure described for the preparation of 7b to afford 14.01 g of a colorless oil. Intermediate 8e was prepared from 7e (12.35 g, 42.34 mmoles, 1 eq) and pentafluorophenol (8.57 g, 46.58 mmoles, 1.1 eq) in 100 mL of anhydrous dichloromethane with triethylamine (6.49 mL, 46.58 mmoles, 1.1 eq) as base according to the procedure described for the preparation of 8b to afford 8.28 g (45%) of a white solid and as a single diastereomer. ¹H NMR (400 MHz, DMSO-d₆) δ 7.48-7.38 (m, 2H), 7.26 (dddt, J=9.8, 7.7, 2.3, 1.1 Hz, 3H), 6.89 (ddd, J=13.9, 9.9, 6.4 Hz, 1H), 4.12-4.04 (m, 2H), 4.04-3.92 (m, 1H), 1.30 (ddd, J=7.1, 5.0, 1.2 Hz, 3H), 1.17 (t, J=7.1 Hz, 3H); ¹⁹F NMR (376 MHz, DMSO-d₆) δ_{F} δ−153.69-−154.18 (m, 2F), −160.26-−160.44 (m, 1F), −163.0426-−163.27 (m, 2F); ³¹P NMR δ_{P} 0.33; LCMS m/z 440 (M+H)⁺.

### (ii) Preparation of 9e

The final target 9e was prepared from 5 (200 mg, 0.661 mmoles, 1.0 eq), 8e (348 mg, 0.793 mmoles, 1.2 eq), and a 1M (in hexanes) of dimethylaluminum chloride (0.330 mL, 0.330 mmoles, 0.50 eq) in 1.5 mL of anhydrous pyridine according to the procedure described for the preparation of 9a to afford, after purification by flash chromatography (40 g silica column, 100-90% dichloromethane in methanol, gradient elution), 46 mg (12%) of a white solid as a mixture of diastereomers (69:31). ¹H NMR (400 MHz, DMSO-d₆) δ 7.61 (s, 2H), 7.45-7.32 (m, 2H), 7.27-7.14 (m, 4H), 6.64 (dd, J=3.7, 2.9 Hz, 1H), 6.49 (ddd, J=16.9, 7.5, 4.3 Hz, 1H), 6.13-5.99 (m, 2H), 5.24-5.09 (m, 1H), 4.41 (dtd, J=18.7, 5.0, 3.3 Hz, 1H), 4.34-4.10 (m, 2H), 4.09-3.98 (m, 3H), 3.90-3.78 (m, 1H), 1.23 (ddd, J=8.3, 7.1, 1.0 Hz, 3H), 1.15 (m, 3H); ¹⁹F NMR (376 MHz, DMSO-d₆) δ_{F}-198.30 to −198.56 (m, 1F); ³¹P NMR δ_{P} 3.66; LCMS m/z 558 (M+H)⁺; HRMS calc for C₂₂H₂₆ClFN₅O₇P.H, 558.13152, found, 558.13053; HPLC 96.3% at 254 nm.

### vi. Synthesis of methyl ((((2R,3R,4S,5R)-5-(4-amino-2-chloro-7H-pyrrolo[2,3-d]pyrimidin-7-yl)-4-fluoro-3-hydroxytetrahydro-furan-2-yl)methoxy)-(phenoxy)phosphoryl)-L-alaninate (9f)

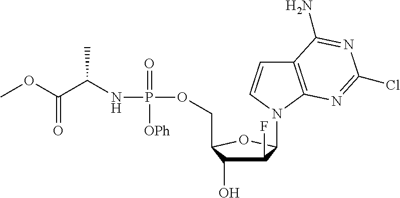

### (i) preparation of methyl((perfluorophenoxy)-(phenoxy)-phosphoryl)-L-alaninate (9f)

Intermediate 7f was prepared from commercial methyl-L-alaninate hydrochloride 6f (10.0 g, 71.64 mmoles, 1.0 eq) and phenyl phosphorodichloridate (11.75 mL, 78.08 mmoles, 1.1 eq) in 140 mL of anhydrous dichloromethane with triethylamine (20.97 mL, 150.45 mmoles, 2.1 eq) as base according to the procedure described for the preparation of 7b to afford 21.34 g of a yellow oil. Intermediate 8f was prepared from 7f (19.89 g, 71.64 mmoles, 1.0 eq) and pentafluorophenol (14.51 g, 78.80 mmoles, 1.1 eq) in 120 mL of anhydrous dichloromethane with triethylamine (10.98 mL, 78.80 mmoles, 1.1 eq) as base according to the procedure described for the preparation of 8b to afford 8.39 g (28%) of a white solid and as a single diastereomer. 1H NMR (400 MHz, DMSO-d6) δ 7.48-7.39 (m, 2H), 7.30-7.21 (m, 3H), 6.91 (dd, J=14.1, 9.9 Hz, 1H), 4.01 (ddq, J=10.9, 9.9, 7.1 Hz, 1H), 3.61 (s, 3H), 1.29 (dd, J=7.1, 1.2 Hz, 3H); 19F NMR (376 MHz, DMSO-d6) δF δ−153.39-−154.18 (m, 2F), −160.05-−160.77 (m, 1F), −163.19 (td, J=23.2, 3.6 Hz, 2F); ³¹P NMR δ_{P} 0.35; LCMS m/z 426 (M+H)⁺.

### (ii) Preparation of 9f

The final target 9f was prepared from 5 (100 mg, 0.330 mmoles, 1.0 eq), 8f (168 mg, 0.0.396 mmoles, 1.2 eq), and 1M (in hexanes) of dimethylaluminum chloride (0.165 mL, 0.165 mmoles, 0.50 eq) in 1.5 mL of anhydrous pyridine according to the procedure described for the preparation of 9a to afford, after purification by flash chromatography (40 g silica column, 100-90% dichloromethane in methanol, gradient elution), 62 mg (34%) of a white foamy solid as a single diastereomer. ¹H NMR (400 MHz, DMSO-d₆) δ 7.61 (s, 2H), 7.43-7.32 (m, 2H), 7.28-7.13 (m, 4H), 6.63 (ddd, J=3.6, 3.1, 0.4 Hz, 1H), 6.49 (ddd, J=16.8, 7.4, 4.4 Hz, 1H), 6.16-6.02 (m, 2H), 5.18 (dddd, J=52.4, 8.9, 4.4, 3.4 Hz, 1H), 4.47-4.35 (m, 1H), 4.34-4.13 (m, 2H), 4.04 (m, 1H), 3.93-3.79 (m, 1H), 3.59 (d, J=3.6 Hz, 3H), 1.27-1.20 (m, 3H): ¹⁹F NMR (376 MHz, DMSO-d₆) δ_{F}−198.33 to −198.62 (m, 1F); ³¹P NMR δ_{P} 3.62; LCMS m/z 544 (M+H)⁺; HRMS calc for C₂₁H₂₄ClFN₅O₇P.H, 544.11587, found, 544.11565; HPLC 96.5% at 254 nm.

### d. Procedure for the Synthesis of 7-Deaza-2′-deoxy-2′-fluoroarabinosyl Nucleoside Prodrugs

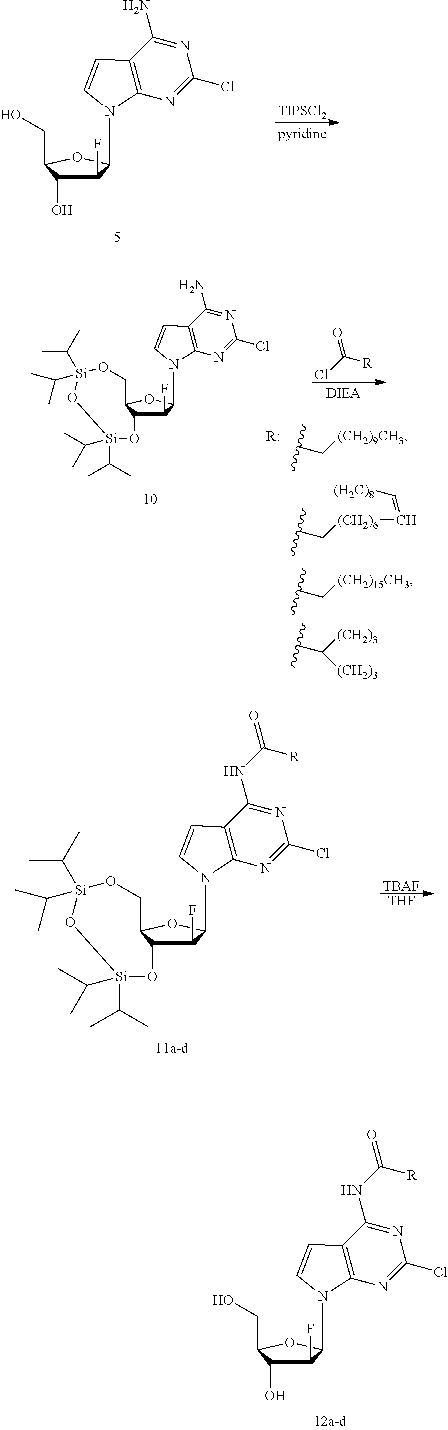

### i. Preparation of N-(2-chloro-7-((6aR,8R,9S,9aR)-9-fluoro-2,2,4,4-tetraisopropyltetrahydro-6H-furo[3,2f][1,3,5,2,4]trioxadisilocin-8-yl)-7H-pyrrolo[2,3-d]pyrimidin-4-yl)dodecanamide (10)

To a solution of 5 (100 mg, 0.330 mmoles, 1.0 eq) in anhydrous pyridine (3.0 mL) was added TIPS-Cl (0.116 mL, 0.363 mmoles, 1.1 eq). The reaction mixture was stirred at 20° C. for 18 hrs. After that time, the reaction mixture was evaporated under reduced pressure to afford a yellow residue, which was dissolved in dichloromethane (50 mL), washed with NH₄Cl (2×30 mL), followed by brine (30 mL). The organic layer was separated, dried (Na₂SO₄), filtered, and then the filtrate was evaporated in vacuo to give a yellow oil. Purification by flash chromatography (24 g silica column, 100-70% hexane in ethyl acetate, gradient elution) provided 151 mg (84%) of 10 as a white foamy solid. ¹H NMR (CDCl₃) δ 7.26 (dd, J=3.8, 2.3 Hz, 1H), 6.56 (dd, J=12.6, 5.0 Hz, 1H), 6.40 (d, J=3.8 Hz, 1H), 5.40 (s, 2H), 5.29-5.13 (m, 1H), 4.68 (ddd, J=23.3, 7.1, 4.5 Hz, 1H), 4.17-4.00 (m, 2H), 3.85 (dddd, J=7.1, 5.3, 3.6, 1.0 Hz, 1H), 1.21-1.03 (m, 28H); ¹⁹F NMR δ_{F}−197.14 to −197.38 (m, 1F); LCMS m/z 545 (M+H)⁺.

### i. Synthesis of N-(2-chloro-7-((2R,3S,4R,5R)-3-fluoro-4-hydroxy-5-(hydroxymethyl)tetrahydrofuran-2-yl)-7H-pyrrolo[2,3-d]pyrimidin-4-yl)dodecanamide (12a)

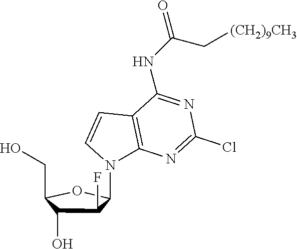

### (i) Preparation of N-(2-chloro-7-((6aR,8R,9S,9aR)-9-fluoro-2,2,4,4-tetraisopropyltetrahydro-6H-furo[3,2-F][1,3,5,2,4]trioxadisilocin-8-yl)-7H-pyrrolo[2,3-d]pyrimidin-4-yl)dodecanamide (11a)

To a solution of the 10 (320 mg, 0.587 mmoles, 1 eq) in anhydrous dichloromethane (2.0 mL) was added DIEA (0.113 mL, 0.646 mmoles, 1.1 eq), followed by a solution of the acid chloride (141 mg, 0.646 mmoles, 1.1 eq) in anhydrous dichloromethane (1.0 mL). The reaction mixture was irradiated with microwaves at 120° C. for 2 hrs. The reaction mixture was evaporated under reduced pressure to afford a brown oil, which was purified by flash chromatography (40 g silica column, 100-70% hexane in EtOAc, gradient elution) to provide 183 mg (43%) of 11a a light yellow solid.

### (ii) Preparation of 12a

To a cold (4° C.) solution of 11a (49 mg, 0.067 mmoles, 1.0 eq) in anhydrous tetrahydrofuran (2.0 mL) was added a 1M solution of tetrabutylammonium fluoride (0.168 mL, 0.168 mmoles, 2.5 eq) in tetrahydrofuran. The reaction mixture was stirred for 18 hrs as it warmed to 20° C. The reaction mixture was evaporated under reduced pressure to afford a residue, which was purified by flash chromatography (24 g silica column 100-95% dichloromethane in methanol, gradient elution) to provide 22 mg (67%) of 12a as a colorless tacky solid. ¹H NMR (DMSO-d6) δ 11.06 (s, 1H), 7.58 (dd, J=3.9, 2.2 Hz, 1H), 6.90 (d, J=3.8 Hz, 1H), 6.59 (dd, J=14.7, 4.6 Hz, 1H), 5.95 (d, J=5.0 Hz, 1H), 5.31-5.12 (m, 1H), 5.08 (t, J=5.6 Hz, 1H), 4.39 (dq, J=19.1, 4.3 Hz, 1H), 3.84 (q, J=4.9 Hz, 1H), 3.75-3.58 (m, 2H), 1.62 (t, J=6.9 Hz, 2H), 1.41-1.13 (m, 18H), 0.94-0.78 (m, 3H); ¹⁹F NMR δ_{F}−197.90 to −198.14 (m, 1F); LCMS m/z 485 (M+H)⁺; HRMS m/z 485.2326 (M+H)⁺; HPLC 95.4% at 254 nm.

### ii. Synthesis of N-(2-chloro-7-((2R,3S,4R,5R)-3-fluoro-4-hydroxy-5-(hydroxymethyl)tetrahydrofuran-2-yl)-7H-pyrrolo[2,3-d]-pyrimidin-4-yl)oleamide (12b)

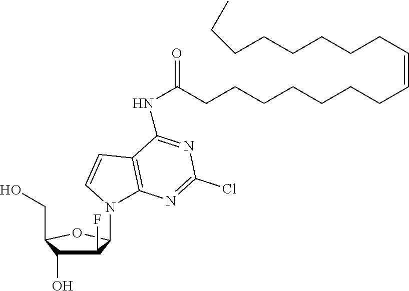

### (i) Preparation of N-(2-chloro-7-((6aR,8R,9S,9aR)-9-fluoro-2,2,4,4-tetraisopropyltetrahydro-6H-furo[3,2-f][1,3,5,2,4]trioxa-disilocin-8-yl)-7H-pyrrolo[2,3-d]pyrimidin-4-yl)oleamide (11b)

Intermediate 11b was prepared from 10 (200 mg, 0.367 mmoles, 1.0 eq) and oleoyl chloride (121 mg, 0.404 mmoles, 1.1 eq) in 2.0 mL of anhydrous dichloromethane with N,N-diisopropylethylamine (0.070 mL, 0.404 mmoles, 1.1 eq) as base according to the procedure described for the preparation of 11a to afford an oil. Purification by flash chromatography (40 g silica column, 100-80% hexane in ethyl acetate, gradient elution) provided 93 mg (31%) of a light yellow oil. ¹H NMR (400 MHz, Chloroform-d) δ 8.05 (s, 1H), 7.40 (dd, J=3.9, 2.0 Hz, 1H), 7.11 (d, J=3.9 Hz, 1H), 6.64 (dd, J=11.1, 5.2 Hz, 1H), 5.41-5.32 (m, 2H), 5.32-5.13 (m, 1H), 4.69 (ddd, J=22.6, 7.3, 4.9 Hz, 1H), 4.15-4.03 (m, 2H), 3.86 (dddd, J=7.4, 4.8, 3.6, 1.0 Hz, 1H), 2.49 (t, J=7.5 Hz, 2H), 2.11-1.95 (m, 4H), 1.76 (p, J=7.4 Hz, 2H), 1.46-1.23 (m, 23H), 1.22-0.96 (m, 30H), 0.94-0.82 (m, 3H); ¹⁹F NMR (376 MHz, Chloroform-d) δ_{F δ-}197.78 to −198.01 (m, 1F); LCMS m/z 809 (M+H)⁺.

### (ii) Preparation of 12b

The final target 12b was prepared from 11b (75 mg, 0.093 mmoles, 1.0 eq) and 1M (in tetrahydrofuran) tetrabutylammonium fluoride (0.232 mL, 0.232 mmoles, 2.5 eq) in 2.0 mL of anhydrous tetrahydrofuran to afford a residue, Purification by flash chromatography (24 g silica column, 100-90% dichloromethane in methanol, gradient elution) provided 41 mg (78%) of a white tacky solid. ¹H NMR (400 MHz, DMSO-d₆) δ 11.06 (s, 1H), 7.58 (dd, J=3.8, 2.2 Hz, 1H), 6.90 (d, J=3.8 Hz, 1H), 6.59 (dd, J=14.7, 4.5 Hz, 1H), 5.95 (d, J=5.1 Hz, 1H), 5.40-5.29 (m, 2H), 5.21 (dt, J=52.8, 4.2 Hz, 1H), 5.09 (t, J=5.6 Hz, 1H), 4.39 (dtd, J=19.1, 5.3, 3.9 Hz, 1H), 3.84 (q, J=4.9 Hz, 1H), 3.75-3.55 (m, 2H), 1.99 (q, J=6.0 Hz, 4H), 1.62 (t, J=7.0 Hz, 2H), 1.42-1.13 (m, 22H), 0.94-0.77 (m, 3H); ¹⁹F NMR (376 MHz, DMSO-d₆) δ_{F}-198.68 to −198.91 (m, 1F); LCMS m/z 567 (M+H)⁺; HRMS calc for C₂₉H₄₄ClFN₄O₄.H, 567.31079, found, 567.31007; HPLC 79.7% at 254 nm.

### iii. Synthesis of N-(2-chloro-7-((2R,3S,4R,5R)-3-fluoro-4-hydroxy-5-(hydroxymethyl)tetrahydrofuran-2-yl)-7H-pyrrolo[2,3-d]pyrimidin-4-yl)stearamide (12c)

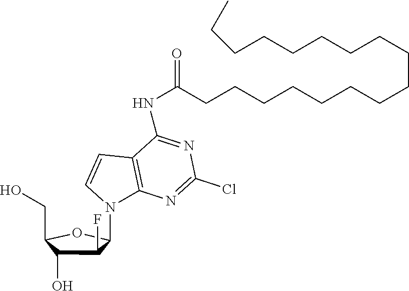

### (i) Preparation of N-(2-chloro-7-((6aR,8R,9S,9aR)-9-fluoro-2,2,4,4-tetraisopropyltetrahydro-6H-furo[3,2-f][1,3,5,2,4]trioxa-disilocin-8-yl)-7H-pyrrolo[2,3-d]pyrimidin-4-yl)stearamide (11c)

Intermediate 11c was prepared from 10 (150 mg, 0.275 mmoles, 1.0 eq) and stearyl chloride (92 mg, 0.303 mmoles, 1.1 eq) in 2.0 mL of anhydrous dichloromethane with N,N-diisopropylethylamine (0.053 mL, 0.303 mmoles, 1.1 eq) as base according to the procedure described for the preparation of 11a to afford a residue. Purification by flash chromatography (40 g silica column, 100-0% hexane in dichloromethane, gradient elution) provided 60 mg (27%) of a colorless tacky solid. ¹H NMR (400 MHz, Chloroform-d) δ 8.34 (s, 1H), 7.40 (dd, J=3.9, 2.0 Hz, 1H), 7.12 (d, J=3.8 Hz, 1H), 6.64 (dd, J=11.1, 5.2 Hz, 1H), 5.32-5.15 (m, 1H), 4.69 (ddd, J=22.7, 7.3, 4.9 Hz, 1H), 4.15-4.02 (m, 2H), 3.86 (dddd, J=7.3, 4.8, 3.6, 1.0 Hz, 1H), 2.50 (t, J=7.5 Hz, 2H), 1.75 (q, J=7.5 Hz, 2H), 1.45-1.23 (m, 28H), 1.19-0.95 (m, 28H), 0.93-0.83 (m, 4H); ¹⁹F NMR (376 MHz, Chloroform-d) δ_{F} δ−197.80 to −198.03 (m, 1F); LCMS m/z 811 (M+H)⁺.

### (ii) Preparation of 12c

The final target 12c was prepared from 11c (46 mg, 0.057 mmoles, 1.0 eq) and 1M (in tetrahydrofuran) tetrabutylammonium fluoride (0.142 mL, 0.142 mmoles, 2.5 eq) in 2.0 mL of anhydrous tetrahydrofuran to afford a residue. Purification by flash chromatography (24 g silica column, 100-95% dichloromethane in methanol, gradient elution) provided 17 mg (52%) of a white waxy solid. ¹H NMR (400 MHz, DMSO-d₆) δ 11.06 (s, 1H), 7.58 (dd, J=3.9, 2.2 Hz, 1H), 6.90 (d, J=3.8 Hz, 1H), 6.59 (dd, J=14.8, 4.6 Hz, 1H), 5.96 (d, J=5.0 Hz, 1H), 5.21 (ddd, J=52.8, 4.7, 3.9 Hz, 1H), 5.09 (t, J=5.7 Hz, 1H), 4.39 (dd, J=19.0, 4.6 Hz, 1H), 3.88-3.80 (m, 1H), 3.67 (dtd, J=17.1, 11.9, 4.9 Hz, 2H), 1.61 (q, J=7.1 Hz, 2H), 1.41-1.20 (m, 30H), 0.92-0.80 (m, 3H); ¹⁹F NMR (376 MHz, DMSO-d₆) δ_{F}-198.68 to −198.91 (m, 1F); HRMS calc for C29H₄₆ClFN₄O₄.H, 569.3264, found, 569.3258; HPLC 97.9% at 254 nm.

### iv. Synthesis of N-(2-chloro-7-((2R,3S,4R,5R)-3-fluoro-4-hydroxy-5-(hydroxymethyl)tetrahydrofuran-2-yl)-7H-pyrrolo[2,3-d]pyrimidin-4-yl)-2-propylpentanamide (12d)

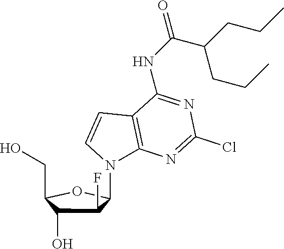

### (i) Preparation of N-(2-chloro-7-((6aR,8R,9S,9aR)-9-fluoro-2,2,4,4-tetra-isopropyltetrahydro-6H-furo[3,2-f][1,3,5,2,4]trioxa-disilocin-8-yl)-7H-pyrrolo[2,3-d]pyrimidin-4-yl)-2-propylpentanamide (11d)

Intermediate 11d was prepared from 10 (150 mg, 0.275 mmoles, 1.0 eq) and 2,2-Di-n-propylacetyl chloride (49 mg, 0.303 mmoles, 1.1 eq) in 2.0 mL of anhydrous dichloromethane with N,N′-diisopropylethylamine (0.053 mL, 0.303 mmoles, 1.1 eq) as base according to the procedure described for the preparation of 11a to afford a residue. Purification by flash chromatography (40 g silica column, 100-80% hexane in ethyl acetate, gradient elution) provided 109 mg (60%) of a colorless oil. ¹H NMR (400 MHz, DMSO-d₆) δ 11.16 (s, 1H), 7.46 (dd, J=3.8, 1.3 Hz, 1H), 6.89 (d, J=3.7 Hz, 1H), 6.62 (dd, J=6.4, 5.3 Hz, 1H), 5.59 (dt, J=54.7, 6.8 Hz, 1H), 4.77 (ddd, J=21.9, 8.6, 7.0 Hz, 1H), 4.15 (ddd, J=12.5, 3.7, 1.7 Hz, 1H), 4.03-3.97 (m, 2H), 3.95-3.88 (m, 1H), 2.77 (tt, J=9.4, 4.9 Hz, 1H), 1.68-0.98 (m, 33H), 0.89 (t, J=7.3 Hz, 9H); ¹⁹F NMR (376 MHz, DMSO-d₆) δ_{F} δ−200.22 to −200.44 (m, 1F); LCMS m/z 671 (M+H)⁺.

### (ii) Preparation of 12d

The final target 12d was prepared from 11d (74 mg, 0.110 mmoles, 1.0 eq) and 1M (in tetrahydrofuran) tetrabutylammonium fluoride (0.276 mL, 0.276 mmoles, 2.5 eq) in 2.0 mL of anhydrous tetrahydrofuran to afford a residue. Purification by flash chromatography (24 g silica column, 100-95% dichloromethane in methanol, gradient elution) provided 40 mg (85%) of a white solid. ¹H NMR (400 MHz, DMSO-d₆) δ 11.13 (s, 1H), 7.59 (dd, J=3.8, 2.2 Hz, 1H), 6.85 (d, J=3.8 Hz, 1H), 6.59 (dd, J=14.6, 4.6 Hz, 1H), 5.95 (d, J=5.1 Hz, 1H), 5.22 (dt, J=52.7, 4.2 Hz, 1H), 5.08 (t, J=5.6 Hz, 1H), 4.46-4.32 (m, 1H), 3.85 (q, J=4.9 Hz, 1H), 3.76-3.56 (m, 2H), 2.77 (tt, J=9.4, 4.9 Hz, 1H), 1.68-1.54 (m, 2H), 1.48-1.22 (m, 6H), 0.90 (td, J=7.3, 1.1 Hz, 6H); ¹⁹F NMR (376 MHz, DMSO-d₆) δ_{F}−198.68 to −198.91 (m, 1F); LCMS: m/z 429 (M+H)⁺; HRMS calc for C₁₉H₂₆ClFN₄O₄.H, 429.1699, found, 429.1704; HPLC 96.7% at 254 nm.

### e. Procedure for the Synthesis of 7-Deaza-2′-deoxy-2′-fluoroarabinosyl Nucleoside Analogs

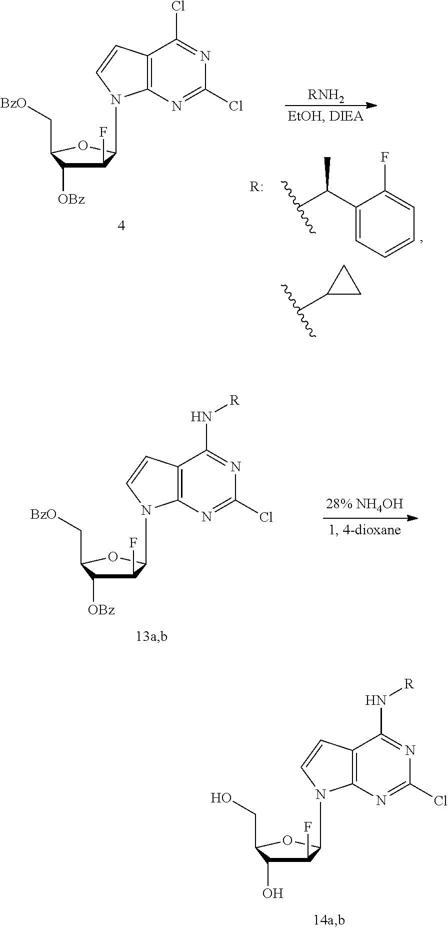

### i. Synthesis of (2R,3R,4S,5R)-5-(2-chloro-4-(((S)-1-(2-fluorophenyl)-ethyl)amino)-7H-pyrrolo[2,3-d]pyrimidin-7-yl)-4-fluoro-2-(hydroxymethyl)tetrahydrofuran-3-ol (14a)

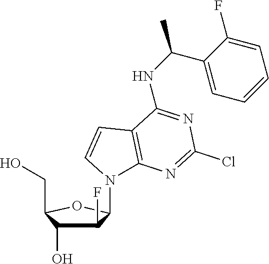

### (i) Preparation of ((2R,3R,4S,5R)-3-(benzoyloxy)-5-(2-chloro-4-(((S)-1-(2-fluorophenyl)ethyl)amino)-7H-pyrrolo[2,3-d]pyrimidin-7-yl)-4-fluorotetrahydrofuran-2-yl)methyl benzoate (13a)

To a mixture of 4 (300 mg, 0.566 mmoles, 1 eq) in 4.0 mL of anhydrous ethanol was added a solution of the S-1-(2-fluorophenyl)ethylamine (94 mg, 0.679 mmoles, 1.2 eq) in 2.0 mL of anhydrous ethanol, followed by N,N′-diisopropylethylamine (0.197 mL, 1.13 mmoles, 2.0 eq). The reaction mixture was stirred in a closed glass high pressure vessel at 80° C. for 18 hrs. The reaction mixture was evaporated under reduced pressure to afford a residue, which was purified by flash chromatography (40 g silica column, 100-0% hexane in ethyl acetate, gradient elution) to provide 195 mg (95%) of 13a as a white foamy solid. ¹H NMR (400 MHz, DMSO-d₆) δ 8.50 (d, J=7.8 Hz, 1H), 8.11 (dd, J=8.2, 1.5 Hz, 2H), 8.06-7.98 (m, 2H), 7.78-7.66 (m, 2H), 7.57 (dt, J=20.0, 7.8 Hz, 4H), 7.46 (td, J=7.7, 1.7 Hz, 1H), 7.35-7.23 (m, 2H), 7.23-7.14 (m, 2H), 6.86 (s, 1H), 6.62 (dd, J=19.5, 3.9 Hz, 1H), 5.88-5.71 (m, 1H), 5.69-5.55 (m, 2H), 4.77 (dd, J=11.6, 3.3 Hz, 1H), 4.72-4.57 (m, 2H), 1.55 (d, J=7.0 Hz, 3H); ¹⁹F NMR (376 MHz, DMSO-d₆) SF 6-119.30 (s, 1F), −198.34 to −198.58 (m, 1F); LCMS m/z 633 (M+H)⁺.

### (ii) Preparation of 14a

To a mixture of 13a (229 mg, 0.362 mmoles, 1.0 eq) in 2.5 mL of 1,4-dioxane was added 28% aqueous ammonium hydroxide (2.5 mL, 18.09 mmoles, 50 eq). The reaction mixture was stirred in a closed glass high pressure vessel at 80° C. for 18 hrs. The reaction mixture was evaporated under reduced pressure to afford an oil, which was purified by flash chromatography (40 g silica column, 100-95% dichloromethane in methanol, gradient elution) to provide 121 mg (79%) of 14a as a white foamy solid. ¹H NMR (400 MHz, DMSO-d₆) δ 8.44 (d, J=7.8 Hz, 1H), 7.46 (td, J=7.7, 1.7 Hz, 1H), 7.31 (ddt, J=13.9, 5.3, 2.4 Hz, 2H), 7.23-7.13 (m, 2H), 6.84 (s, 1H), 6.44 (dd, J=15.3, 4.5 Hz, 1H), 5.89 (d, J=5.1 Hz, 1H), 5.62 (t, J=7.4 Hz, 1H), 5.18 (t, J=4.1 Hz, 1H), 5.11-4.96 (m, 2H), 4.35 (dq, J=18.9, 4.8 Hz, 1H), 3.80 (q, J=4.9 Hz, 1H), 3.65 (qq, J=11.6, 5.2 Hz, 2H), 1.55 (d, J=7.0 Hz, 3H); ¹⁹F NMR (376 MHz, DMSO-d₆) δ_{F}−119.31 (s, 1F), −198.58 to −198.81 (m, 1F); LCMS: m/z 425 (M+H)⁺; HRMS calc for C₁₉H₁₉ClF₂N₄O₃.H, 425.1186, found, 425.1181; HPLC 95.6% at 254 nm.

### ii. Synthesis of (2R,3R,4S,5R)-5-(2-chloro-4-(cyclopropylamino)-7H-pyrrolo[2,3-d]pyrimidin-7-yl)-4-fluoro-2-(hydroxymethyl)-tetrahydrofuran-3-ol (14b)

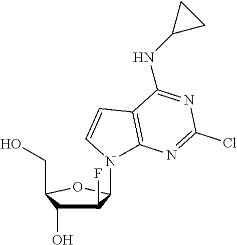

### (i) Preparation of ((2R,3R,4S,5R)-3-(benzoyloxy)-5-(2-chloro-4-(cyclopropylamino)-7H-pyrrolo[2,3-d]pyrimidin-7-yl)-4-fluorotetrahydrofuran-2-yl)methyl benzoate (13b)

Intermediate 13b was prepared from 4 (300 mg, 0.566 mmoles, 1.0 eq) and cyclopropylamine (39 mg, 0.679 mmoles, 1.2 eq) in 4.0 mL of anhydrous ethanol with N,N′-diisopropylethylamine (0.197 mL, 1.13 mmoles, 2.0 eq) as base according to the procedure described for the preparation of 13a to afford a residue. Purification by flash chromatography (24 g silica column, 100-0% hexane in ethyl acetate, gradient elution) provided 214 mg (69%) as a white foamy solid. ¹H NMR (400 MHz, DMSO-d₆) δ 8.17 (s, 1H), 8.11-8.04 (m, 2H), 8.03-7.94 (m, 2H), 7.75-7.62 (m, 2H), 7.61-7.46 (m, 4H), 7.21 (t, J=3.5 Hz, 1H), 6.61 (dd, J=19.9, 3.9 Hz, 2H), 5.83-5.56 (m, 2H), 4.73 (dd, J=11.6, 3.3 Hz, 1H), 4.69-4.57 (m, 2H), 2.91 (dq, J=7.1, 3.5 Hz, 1H), 0.78 (s, 2H), 0.57 (d, J=3.3 Hz, 2H); ¹⁹F NMR (376 MHz, DMSO-d₆) δ_{F δ-}198.39 to −198.53 (m, 1F); LCMS m/z 551 (M+H)⁺.

### (ii) Preparation of 14b

The final target 14b was prepared from 13b (204 mg, 0.370 mmoles, 1.0 eq) and 28% aqueous ammonium hydroxide (2.5 mL, 18.5 mmoles, 50 eq) in 2.5 mL of 1,4-dioxane according to the procedure described for the preparation of 14a to afford a residue. Purification by flash chromatography (24 g silica column, 100-95% dichloromethane in methanol, gradient elution) provided 99 mg (78%) of a white foamy solid. ¹H NMR (400 MHz, DMSO-d₆) δ 8.14 (s, 1H), 7.30 (s, 1H), 6.67 (s, 1H), 6.45 (dd, J=15.5, 4.5 Hz, 1H), 5.90 (d, J=5.1 Hz, 1H), 5.23-4.99 (m, 2H), 4.36 (dq, J=19.0, 4.8 Hz, 1H), 3.80 (q, J=5.0 Hz, 1H), 3.64 (ddt, J=17.8, 11.8, 6.1 Hz, 2H), 2.93 (tq, J=7.2, 3.6 Hz, 1H), 0.82 (d, J=7.5 Hz, 2H), 0.59 (t, J=3.3 Hz, 2H); LCMS: m/z 343 (M+H)⁺; HRMS calc for C₁₄H₁₆ClFN₄O₃.H, 343.0968, found, 343.0967; HPLC 100.0% at 254 nm.

### f. Procedure for the Synthesis of 7-Deaza-2′-deoxy-2′-fluoroarabinosyl Nucleoside Analogs

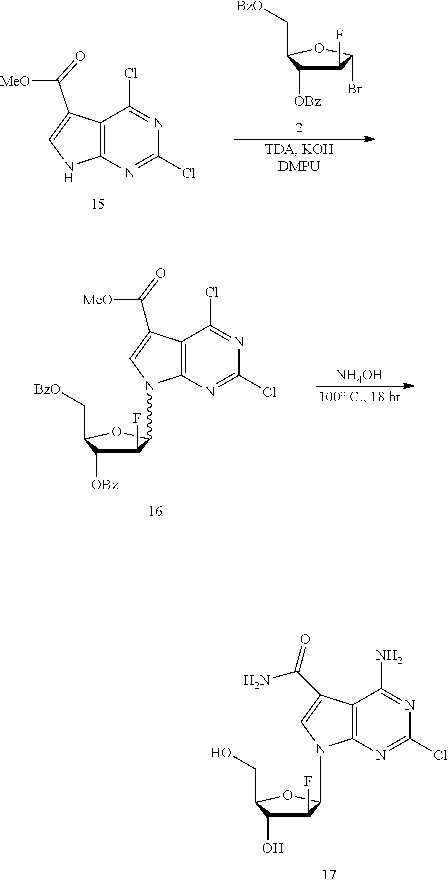

### i. Preparation of methyl 7-((3S,4R,5R)-4-(benzyloxy)-5-((benzyloxy)methyl)-3-fluorotetrahydrofuran-2-yl)-2,4-dichloro-7H-pyrrolo[2,3-d]pyrimidine-5-carboxylate (16)

To a mixture of anhydrous acetonitrile (300 mL) and potassium hydroxide (229 mg, 4.08 mmoles, 2.12 eq) was added cat Tris[2-(2-methoxyethoxy)ethyl]amine (TDA-1) (0.037 mL, 0.115 mmoles, 0.060 eq). The reaction mixture was stirred for 20 min at room temperature and then 15 (500 mg, 1.92 mmoles, 1.0 eq) was added. The reaction mixture was stirred for 30 min at room temperature. 1,3-dimethyl-3,4,5,6-tetrahydro 2(1H)-pyrimidone (0.400 mL) was added to increase solubility and then the reaction mixture was stirred for an additional 1.5 hrs. A solution of 2 (976 mg, 2.31 mmoles, 1.2 eq) in 15 mL of anhydrous acetonitrile was added and then the reaction mixture was stirred at 20° C. for 3 days. The cloudy reaction mixture was evaporated under reduced pressure to afford an orange semi-solid, which was quenched with ammonium chloride (50 mL) and then extracted with ethyl acetate (3×50 mL). The organic extracts were combined and washed with brine (50 mL). The organic layer was separated, dried (sodium sulfate), filtered, and then the filtrated was evaporated under reduced pressure to give an oil. Purification by flash chromatography (40 g silica column, 100-70% hexane in ethyl acetate, gradient elution) provided 521 mg (46%) of 16 as a white solid and as a mixture of anomers (beta:alpha 9:1). ¹H NMR (400 MHz, DMSO-d₆) δ 8.40 (d, J=2.7 Hz, 1H), 8.16-8.09 (m, 2H), 8.08-7.99 (m, 2H), 7.79-7.66 (m, 2H), 7.65-7.46 (m, 4H), 6.90 (dd, J=18.0, 4.0 Hz, 1H), 5.96-5.67 (m, 2H), 4.89-4.71 (m, 3H), 3.78 (d, J=0.5 Hz, 3H); Beta Anomer: ¹⁹F NMR (376 MHz, DMSO-d₆) δ_{F} δ−197.30 to −197.54 (m, 1F); Alpha Anomer: ¹⁹F NMR (376 MHz, DMSO-d₆) δ_{F} δ−187.87 to −188.09 (m, 1F); LCMS m/z 588 (M+H)⁺.

### ii. Preparation of 17

The final target 17 was prepared from 16 (82 mg, 0.144 mmoles, 1.0 eq) and 28% aqueous ammonium hydroxide (5.0 mL, 36.7 mmoles, 255 eq) in 5.0 mL of 1,4-dioxane according to the procedure described for the preparation of 14a,b to afford a solid. Purification by flash chromatography (24 g silica column, 100-90% dichloromethane in methanol, gradient elution) provided 17 mg (34%) of 17 as white solid. ¹H NMR (400 MHz, DMSO-d₆) δ 9.38 (s, 1H), 8.13 (s, 1H), 8.10 (d, J=2.1 Hz, 1H), 7.91 (s, 1H), 7.49 (s, 1H), 6.48 (dd, J=16.8, 4.2 Hz, 1H), 5.97 (d, J=5.1 Hz, 1H), 5.15 (ddd, J=52.3, 4.2, 3.1 Hz, 1H), 4.98 (t, J=5.8 Hz, 1H), 4.35 (dtd, J=18.1, 4.8, 3.2 Hz, 1H), 3.90-3.83 (m, 1H), 3.73-3.61 (m, 2H); ¹⁹F NMR (376 MHz, DMSO-d₆) δ_{F}−198.07 to −198.30 (m, 1F); LCMS: m/z 346 (M+H)⁺; HRMS calc for C₁₂H₁₃ClFN₅O₄.H, 346.0713, found, 346.0718; HPLC 93.3% at 254 nm.

### 2. Biology Experimentals

### a. Cell Lines: HepG2 2.2.15 Cells

The HepG2 2.2.15 cell line is a stable human hepatoblastoma cell line that contains two copies of the HBV wild-type strain ayw1 genome and constitutively produces high levels of HBV. Cells were sub-cultured twice a week in Dulbecco's Modified Eagle's Medium (DMEM) supplemented with 10% FBS, 380 μg/mL G418, 2.0 mM L-Glutamine, 100 units/mL Penicillin, and 100 μg/mL Streptomycin. Total cell number and percent viability determinations were performed using a hemacytometer and trypan blue dye exclusion prior to each experiment set-up. Cell viability was always greater than 95% for experiment set-ups.

### b. HepG2 2.2.15 Cell-Based HBV Replication Assay

The primary anti-HBV assay was performed as previously described (Korba and Milman (1991) Antiviral Res. 15: 217-228; Korba and Gerin (1992) Antiviral Res. 19: 55-70) with modifications to use real-time PCR (TaqMan-based) to measure extracellular HBV DNA virion-associated released from HepG2 2.2.15 cells. Antiviral compounds blocking any late step of viral replication such as transcription, translation, pre-genome encapsidation, reverse transcription, particle assembly and release can be identified and characterized using this cell line. Briefly, HepG2 2.2.15 cells were seeded in 96-well microtiter plates at 1.5×10⁴ cells/well in Dulbecco's Modified Eagle's Medium supplemented with 2% FBS, 2.0 mM L-Glutamine, 100 units/mL Penicillin, and 100 μg/mL Streptomycin. Lamivudine (3TC) was used as the positive control for anti-HBV activity, while media alone was added to cells as the untreated virus replication control. Three days post treatment with test article (DPV), cell culture medium was replaced with fresh medium containing the appropriately diluted test compounds. Six days following the initial administration of the test compounds, the cell culture supernatant was collected, treated with pronase and then used in a real-time TaqMan-based PCR assay. The PCR-amplified HBV DNA was detected by measuring fluorescent signal resulting from the exonucleolytic degradation of a quenched fluorescent probe molecule that hybridizes to the amplified HBV DNA.

### c. Evaluation of Cell Viability

At experiment conclusion, MTS reagent (CellTiter®96 Reagent, Promega) reagent was added to culture wells (96 well microtiter plates) and permitted to incubate with cells for 2-4 hours at 37° C., 5% CO₂. Each cell culture well was measured for MTS reagent reduction (color change, 490/650 nm) using a SpectraMax i3 plate reader (Molecular Devices).

### d. Evaluation of Compound 5 Against HBV in HepG2 2.2.15 Cells

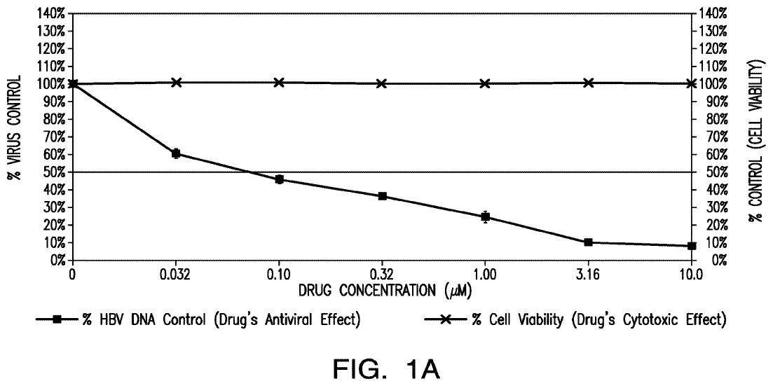

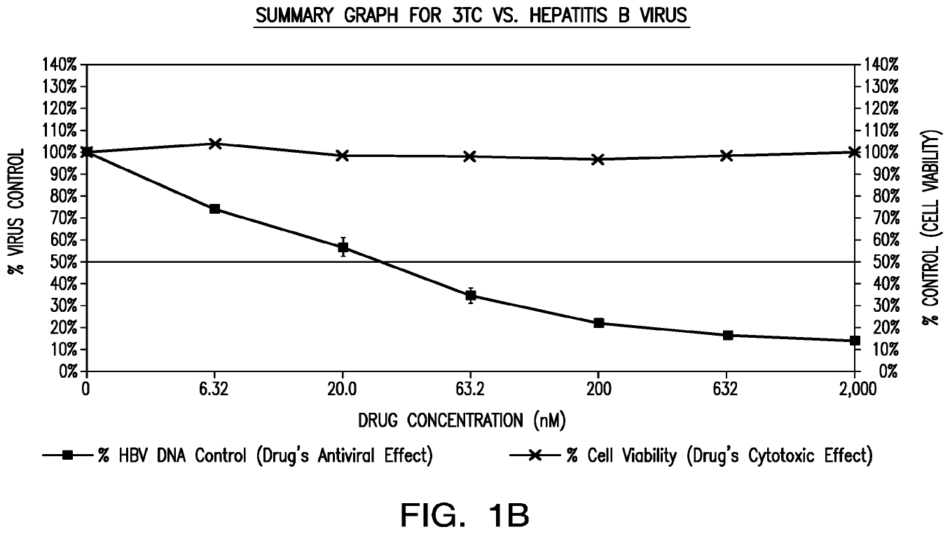

The antiviral activity of compound 5 compared to 3TC against HBV in HepG2 2.2.15 cells is shown in Table 1 below. See also FIG. 1A and FIG. 1B.

TABLE 1

| | High-Test | | | | Selectivity | | |
|---|---|---|---|---|---|---|---|
| | Concen- | EC₅₀ | EC₉₀ | CC₅₀ | Index (SI₅₀) | | |
| No. | tration (μM) | (μM) | (μM) | (μM) | (CC₅₀/EC₅₀) | SI₉₀ | Activity |
| 5 | 10 | 0.07 | 1.28 | >10 | >167 | >3 | Hi |
| 3TC | 2 | 0.029 | 0.511 | >2 | >70 | >3 | Hi |

### e. Evaluation of Compound 5 Against HSV-1 Strain HF in Vero Cells

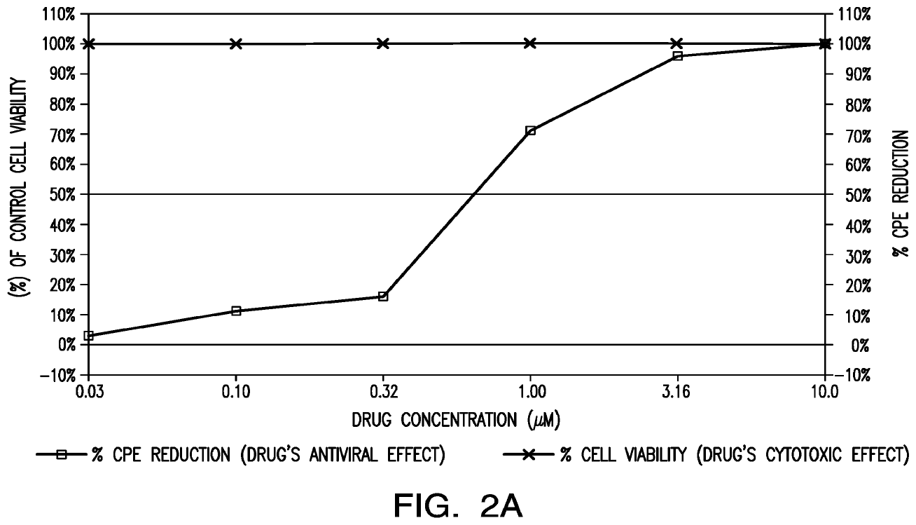

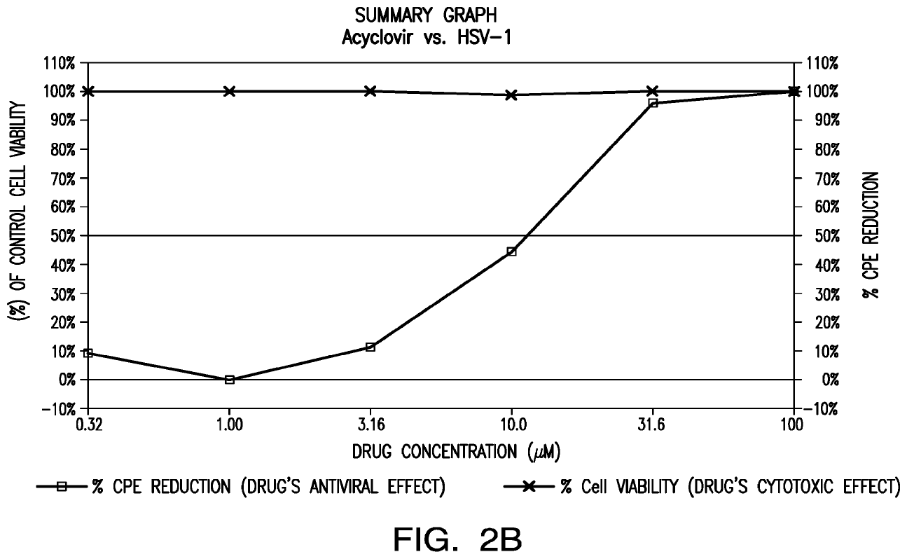

The antiviral activity of compound 5 compared to acyclovir against HSV-1 Strain HF in Vero cells is shown in Table 2 below. See also FIG. 2A and FIG. 2B.

TABLE 2

| | High-Test | IC₅₀ | TC₅₀ | Selectivity Index |
|---|---|---|---|---|
| No. | Concentration (μM) | (μM) | (μM) | (SI₅₀) (TC₅₀/IC₅₀) |
| 5 | 10 | 0.65 | >10 | >15.4 |
| Acyclovir | 100 | 11.4 | >100 | >8.77 |

### 3. Characterization of Antiviral Agents

A list of compounds evaluated for antiviral activity in a HBV virus yield assay is shown in Table 3 below.

TABLE 3

| | | EC₅₀ | EC₉₀ | CC₅₀ |
|---|---|---|---|---|
| No. | Structure | (μM) | (μM) | (μM) |
| 3TC | | 0.029 | 0.511 | >2 |
| (control) | | | | |
| 5 | 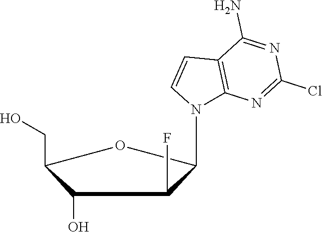 | 0.06 | 1.28 | >10 |
| 9a | 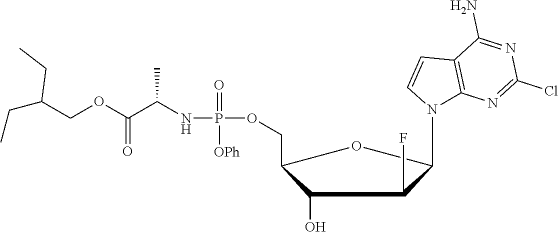 | 0.16 | 1.58 | >10 |
| 9b | 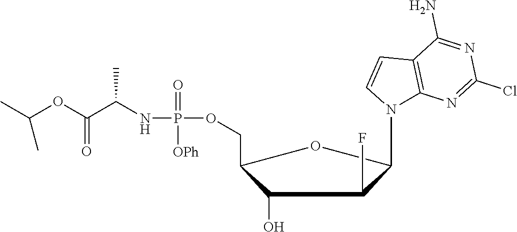 | 0.09 | 1.05 | >10 |
| 9c | 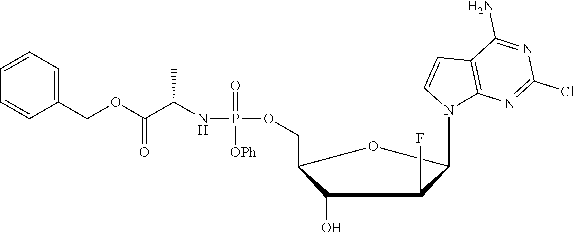 | 0.24 | >10 | >10 |
| 9d | 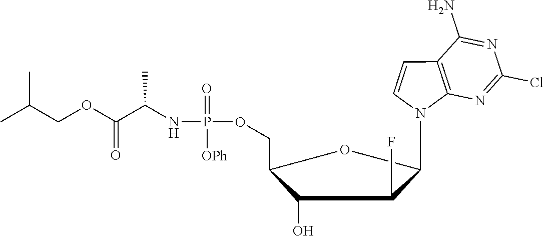 | 0.30 | >10 | >10 |
| 9e | 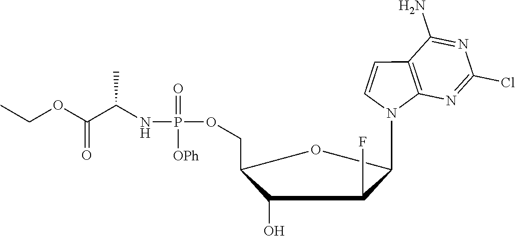 | 0.14 | >10 | >10 |
| 9f | 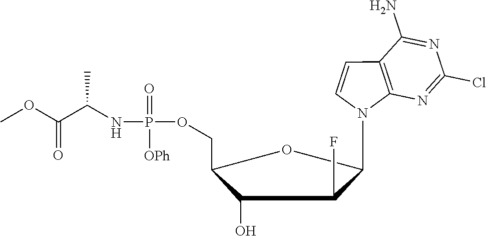 | 0.24 | >10 | >10 |
| 12a | 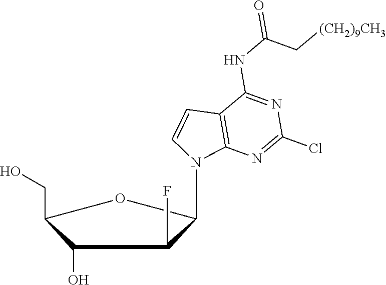 | 0.08 | 0.95 | >10 |
| 12b | 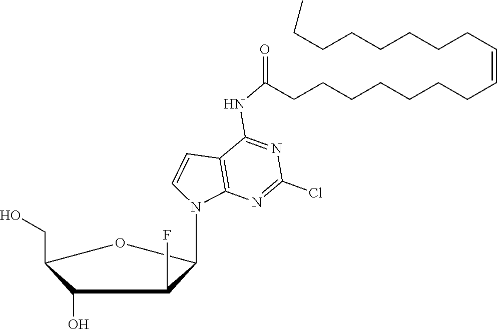 | 0.13 | >10 | >10 |
| 12c | 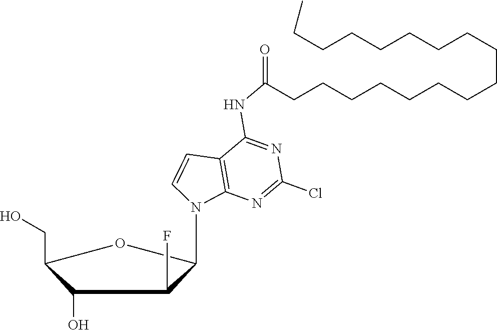 | 0.53 | >10 | >10 |
| 12d | 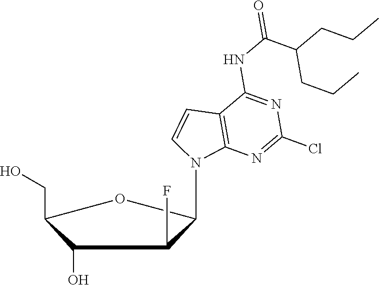 | 2.12 | >10 | >10 |
| 14a | 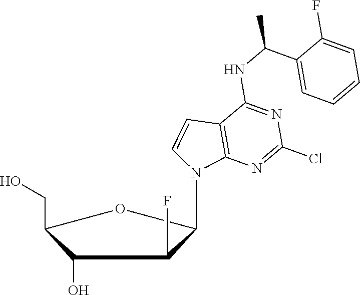 | Not tested | Not tested | Not tested |
| 14b | 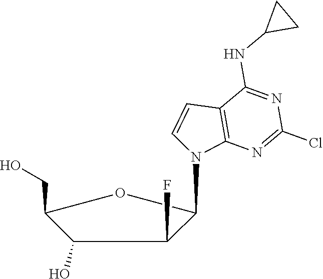 [SMILES: OC[C@H]1O[C@@H](n2ccc3c(NC4CC4)nc(Cl)nc32)[C@@H](F)[C@@H]1O] | Not tested | Not tested | Not tested |
| 17 | 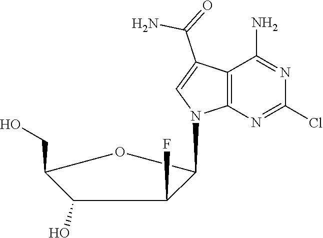 [SMILES: NC(=O)c1cn([C@@H]2O[C@H](CO)[C@@H](O)[C@@H]2F)c2nc(Cl)nc(N)c12] | Not tested | Not tested | Not tested |

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A compound having a structure represented by a formula: 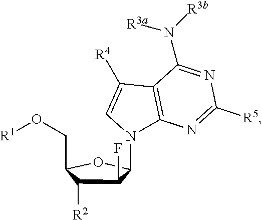
wherein R¹ is selected from hydrogen, —C(O)R¹⁰, —P(O)(OR¹¹)₂, and —P(O)(OR¹¹)R¹²;
wherein R² is selected from hydrogen, —OH, C1-C8 alkoxy, —P(O)(OR^{11′})₂, and —P(O)(OR^{11′})R^{12′};
wherein R¹⁰, when present, is selected from C1-C30 alkyl, C2-C30 alkenyl, and —CH(NH₂)R²⁰;
wherein R²⁰, when present, is selected from hydrogen, methyl, isopropyl, isobutyl, sec-butyl, —(CH₂)₃NHC(NH)NH₂, —(CH₂)₄NH₂, —CH₂CO₂H, —(CH₂)₂CO₂H, —CH₂OH, —CH(OH)CH₃, —CH₂C(O)NH₂, —(CH₂)₂C(O)NH₂, —CH₂SH, —(CH₂)₂SCH₃, —CH₂SeH, —CH₂C₆H₅, and —CH₂Cy¹;
wherein Cy¹, when present, is selected from monocyclic aryl, para-hydroxy monocyclic aryl, 4-imidazolyl, and 3-indolyl;
wherein each of R¹¹ and R^{11′}, when present, is independently selected from hydrogen, C1-C4 alkyl, —(C1-C10 alkyl)CO₂(C1-C10 alkyl), —(C1-C10 alkoxy)CO₂(C1-C10 alkyl), —(C1-C10 alkyl)CO₂(C1-C10 alkylthiol), —(C1-C10 alkyl)-S—S—(C1-C10 alkyl), Ar¹, and —CH₂Ar¹;
wherein each occurrence of Ar¹, when present, is selected from aryl and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl;
wherein each of R¹² and R^{12′}, when present, is selected from —OR²¹ and —NHR²¹;
wherein each occurrence of R²¹, when present, is selected from hydrogen, —(C1-C10 alkyl)CO₂(C1-C10 alkyl), —(C1-C10 alkoxy)CO₂(C1-C10 alkyl), —(C1-C10 alkyl)CO₂(C1-C10 alkylthiol), —(C1-C10 alkyl)-S—S—(C1-C10 alkyl), Ar², —CH₂Ar², —P(O)OHOP(O)(OH)₂, and a structure represented by a formula: 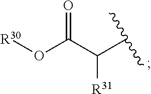
wherein each occurrence of R³⁰, when present, is independently selected from hydrogen, C1-C8 alkyl, Cy², and —CH₂Cy²;
wherein each occurrence of Cy², when present, is independently selected from C3-C6 cycloalkyl, aryl, and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl;
wherein each occurrence of R³¹, when present, is independently selected from hydrogen and C1-C8 alkyl; and
wherein each occurrence of Ar², when present, is independently selected from aryl and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl;
or wherein each of R¹ and R² together comprise a structure represented by a formula: 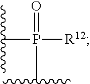
wherein each of R³ and R^{3b} is independently selected from hydrogen, —OH, C1-C10 alkoxy, C1-C8 alkyl, —C(O)(C1-C30 alkyl), —C(O)(C2-C30 alkenyl), Cy³, —CR^{32a}R^{32b}Ar³;
wherein each of R^{32a} and R^{32b}, when present, is independently selected from hydrogen and C1-C4 alkyl;
wherein Cy³, when present, is C3-C6 cycloalkyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl;
wherein Ar³, when present, is selected from aryl and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl;
wherein R⁴ is selected from hydrogen, halogen, —CN, —C(O)NH₂, —CO₂H, —COMe, —SO₂Me, C1-C4 haloalkyl, and Ar⁴;
wherein Ar⁴, when present, is selected from aryl and heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl;
wherein R⁵ is selected from halogen, —CF₃, C1-C10 alkyl, and Ar⁵; and
wherein Ar⁵, when present, is selected from aryl and heteroaryl, and is substituted with 0, 1, 2, or 3 groups halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl,
or a pharmaceutically acceptable salt thereof.

2. The compound of claim 1, wherein R¹ is hydrogen.

3. The compound of claim 1, wherein R¹ is —P(O)(OR¹¹)R¹².

4. The compound of claim 3, wherein R¹² is —NHR²¹.

5. The compound of claim 1, wherein R² is selected from hydrogen and —OH.

6. The compound of claim 1, wherein each of R^{3a} and R^{3b} is independently selected from hydrogen and Cy³.

7. The compound of claim 1, wherein R⁴ is hydrogen.

8. The compound of claim 1, wherein R⁵ is —Cl.

9. The compound of claim 1, wherein the compound has a structure represented by a formula selected from:

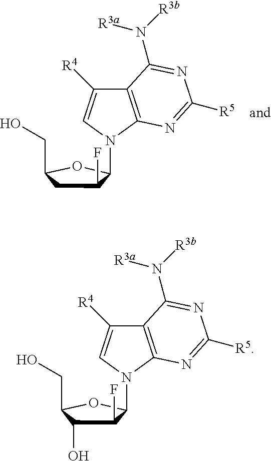

10. The compound of claim 1, wherein the compound has a structure represented by a formula:

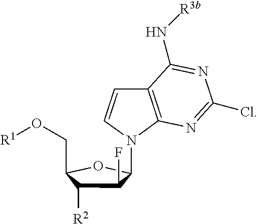

11. The compound of claim 1, wherein the compound has a structure represented by a formula:

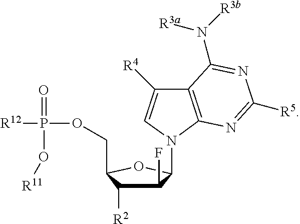

12. The compound of claim 1, wherein the compound has a structure represented by a formula:

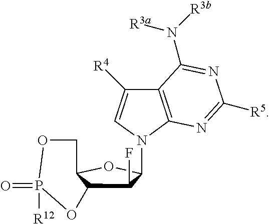

13. The compound of claim 1, wherein the compound has a structure represented by a formula:

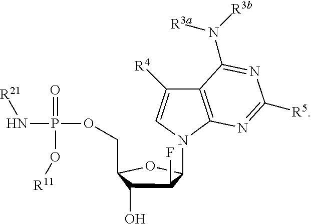

14. The compound of claim 1, wherein the compound has a structure represented by a formula:

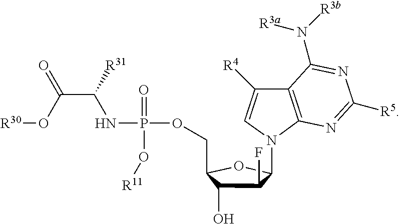

15. The compound of claim 1, wherein the compound is selected from:

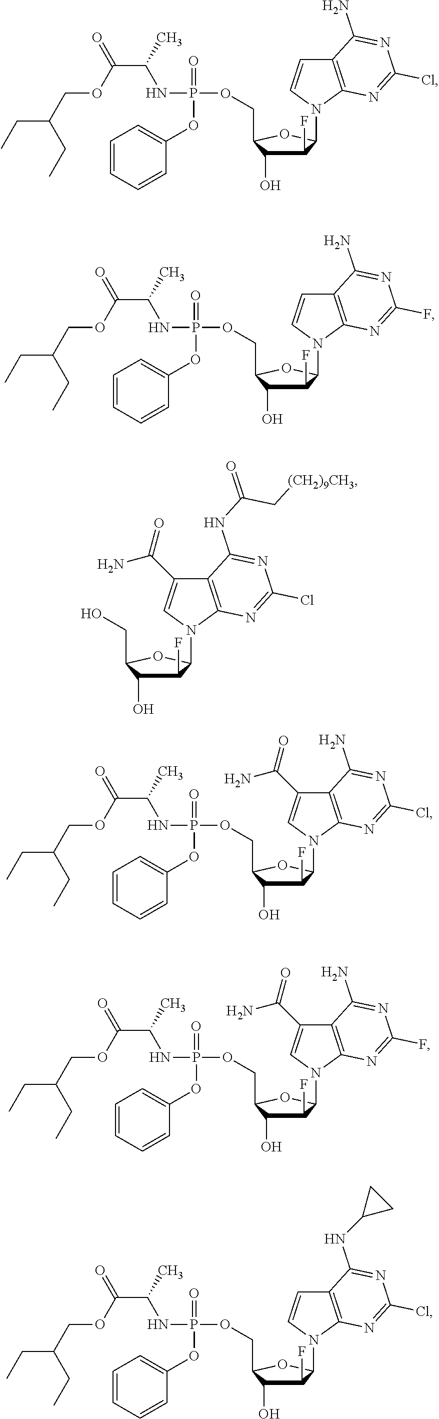

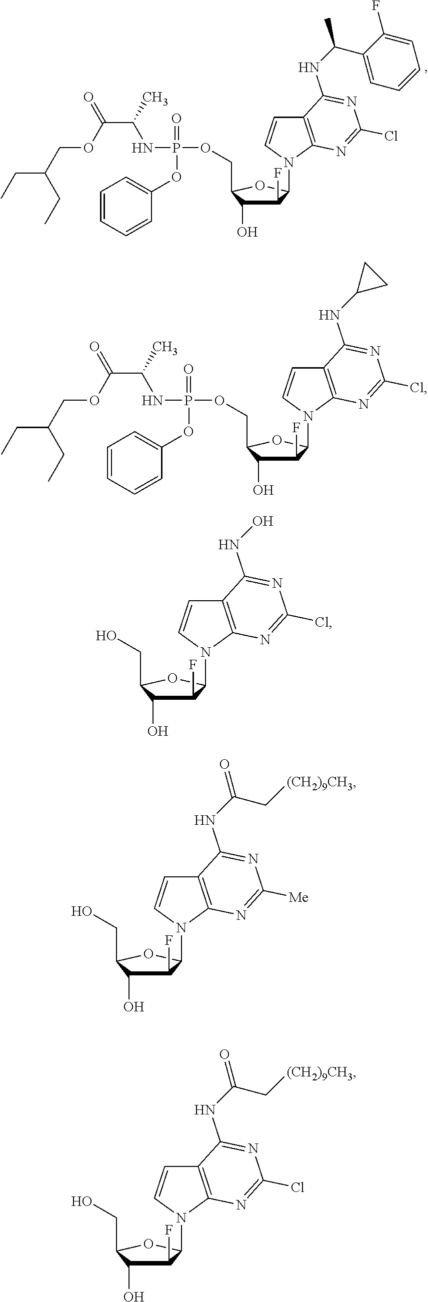

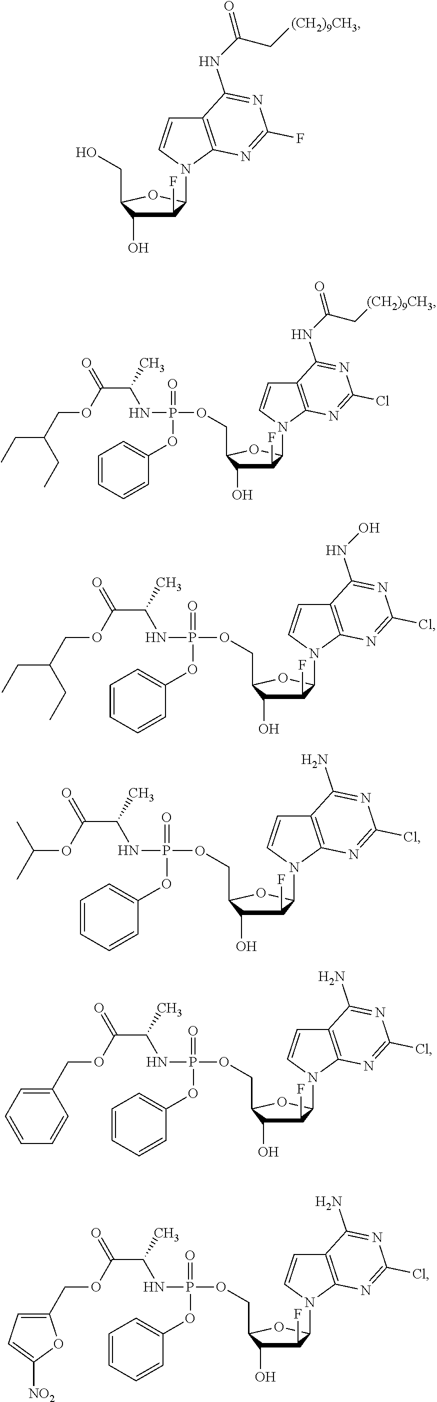

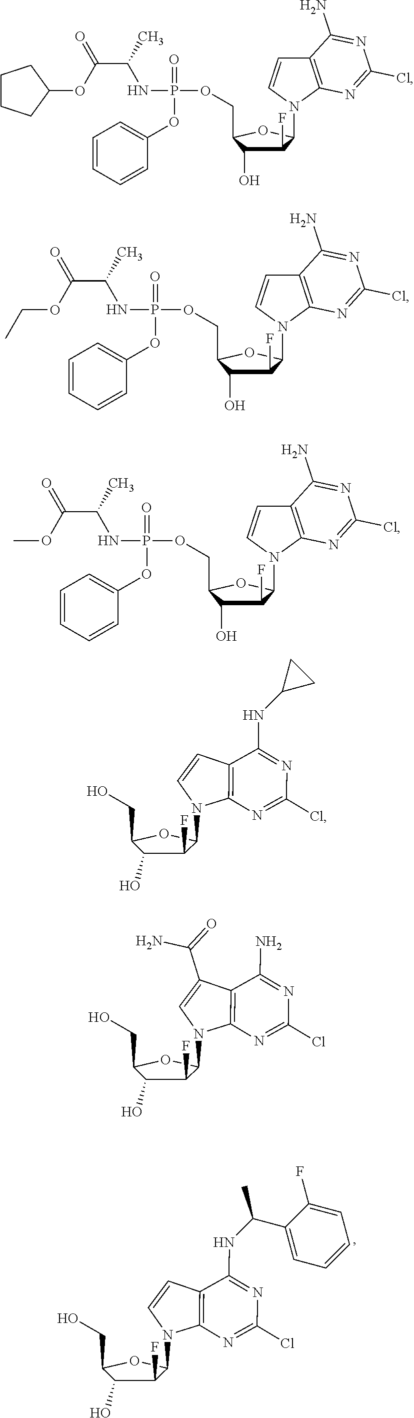

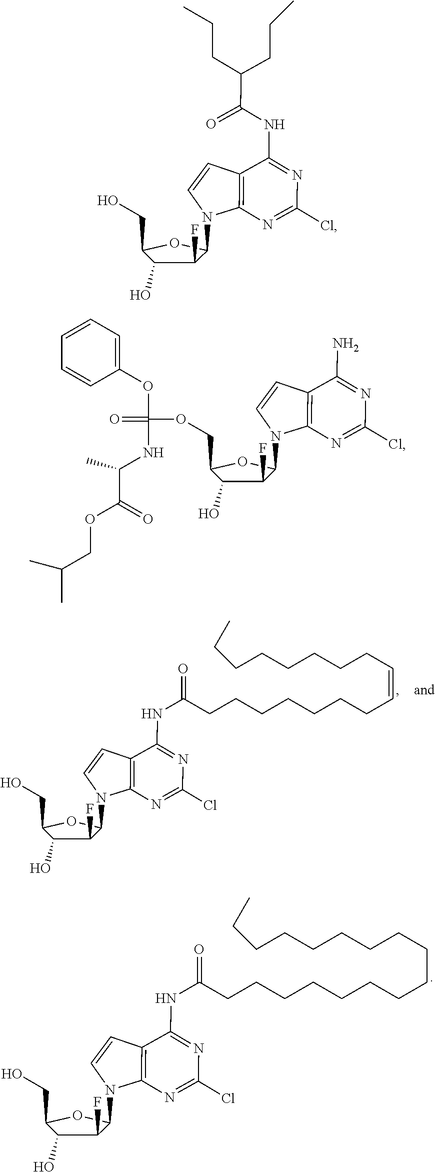

16. A pharmaceutical composition comprising a therapeutically effective amount of the compound of claim 1 and a pharmaceutically acceptable carrier.

17. A method of treating a viral infection in a subject, the method comprising the step of administering to the subject an effective amount of the compound of claim 1.

18. The method of claim 17, wherein the viral infection is selected from human immunodeficiency virus (HIV), human papillomavirus (HPV), herpes simplex virus (HSV), human cytomegalovirus (HCMV), chicken pox, infectious mononucleosis, mumps, measles, rubella, shingles, ebola, viral gastroenteritis, viral hepatitis, viral meningitis, human metapneumovirus, human parainfluenza virus type 1, parainfluenza virus type 2, parainfluenza virus type 3, respiratory syncytial virus, viral pneumonia, Chikungunya virus (CHIKV), Venezuelan equine encephalitis (VEEV), dengue (DENV), influenza, West Nile virus (WNV), zika (ZIKV), 229E, NL63, OC43, HKU1, Middle East respiratory syndrome coronavirus (MERS-CoV), severe acute respiratory syndrome coronavirus (SARS-CoV), and severe acute respiratory syndrome coronavirus disease 2019 (SARS-CoV-2).

19. The method of claim 17, wherein the viral infection is viral hepatitis.

20. The method of claim 19, wherein the viral hepatitis is hepatitis B virus (HBV).
